(19) United States
(12) Patent Application Publication
(10) Pub. No.: US 2011/0038920 A1
(43) Pub. Date: Feb. 17, 2011

(54) WOUND HEALING COMPOSITIONS AND TREATMENTS

(76) Inventors: Ryoichi Mori, Bristol (GB); Paul Martin, Bristol (GB); Bradford James Duft, Rancho Santa Fe, CA (US); Tanya Shaw, Bristol (GB)

Correspondence Address: DUANE MORRIS LLP - San Diego, 101 WEST BROADWAY, SUITE 900, SAN DIEGO, CA, 92101-8285, US

(21) Appl. No.: 12/812,017

(22) PCT Filed: Jan. 7, 2009
(86) PCT No.: PCT/US09/00129

§ 371 Date: Nov. 3, 2010

Related U.S. Application Data
(60) Provisional application No. 61/010,391, filed on Jan. 7, 2008.

(51) Int. Cl. A61K 31/713; C07H 21/02; A61K 9/70; A61P 29/00; A61P 17/02

(52) U.S. Cl. 424/445; 536/24.5; 514/44 A

(57) ABSTRACT

Methods and compositions comprising an anti-ostepontin agent and/or a PDGF receptor blocker or antagonist, alone or in combination with one or more anti-connexin agents, for example, one or more anti-connexin polynucleotides and/or one or more anti-connexin peptides or peptidomimetics, are provided for the promotion and/or improvement of wound healing and/or tissue repair, and for anti-scarring, anti-inflammatory, anti-fibrosis and anti-adhesion indications.

This application is a National Stage Application under 35 U.S.C. §371 of International Application No. PCT/US2009/000129, filed Jan. 7, 2009 which claims the benefit of priority to U.S. Provisional Application No. 61/010,391 filed on Jan. 7, 2008. The disclosures if both are incorporated herein by reference.

### FIELD

The inventions relate to wounds and wound healing, in particular to acute wounds and to wounds that do not heal at expected rates, such as delayed-healing wounds, incompletely healing wounds, chronic wounds, and dehiscent wounds.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance on cellular, tissue, organ, and organism levels. Wound healing, whether initiated by trauma, microbes or foreign materials, proceeds via a complex process encompassing a number of overlapping phases, including inflammation, epithelialization, angiogenesis and matrix deposition. Normally, these processes lead to a mature wound and a certain degree of scar formation. Although inflammation and repair mostly occur along a prescribed course, the sensitivity of the process is dependent on the balance of a variety of wound healing molecules, including for example, a network of regulatory cytokines and growth factors.

One of these molecules is osteopontin (OPN), a protein that is constitutively expressed in normal elastic fibers in human skin and aorta (Baccarini-Contri, M. et al. (1994) Matrix Biol. 14: 553-560). OPN is expressed by wound granulation tissue fibroblasts coincident with the wound inflammatory response. OPN expression is reported to be elevated in lung and liver fibrosis (Pardo, A., K. et al. (2005) PLoS Med 2:e251 and Lee, S. H., et al. (2004) Biochem Pharmacol 68:2367-2378, respectively), and is known to act both as a secreted chemokine-like protein (El-Tanani, M. K., et al. (2006) Cytokine Growth Factor Rev 17:463-474), as well as part of an intracellular signaling complex (Shinohara, M. L., et al. (2006) Nat Immunol 7:498-506.), depending on the cellular context. OPN is reported to have many and diverse cellular effects, including the capacity to regulate cell adhesion, migration and survival, as well as several functions in immune regulation, and a key role in the interplay between osteoblasts and osteoclasts as bone is remodeled (Denhardt, D. T., et al. (2001) J Clin Invest 107:1055-1061). In animal models, OPN expression in wound tissue is inflammation-dependent based on expression profile data using a mouse model (Lisa Cooper, et al. Genome Biol. 6(1): Published online 2004 Dec. 23. doi: 10.1186/gb-2004-6-1-r5)

Gap junctions are cell membrane structures that facilitate direct cell-cell communication. A gap junction channel is formed of two connexons (hemichannels), each composed of six connexin subunits. Each hexameric connexon docks with a connexon in the opposing membrane to form a single gap junction. Gap junction channels are reported to be found throughout the body. Tissue such as the corneal epithelium, for example, has six to eight cell layers, yet expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues have been shown to alter connexin protein expression pattern following injury or transplantation (Qui, C. et al., (2003) Current Biology, 13:1967-1703; Brander et al., (2004), J. Invest Dermatol. 122:1310-20).

It has been reported that abnormal connexin function may be linked to certain disease states (e.g. heart diseases) (A. C. de Carvalho, et al., J Cardiovasc Electrophysiol 1994, 5 686). In certain connexin proteins, alterations in the turnover and trafficking properties may be induced by the addition exogenous agents which may affect the level of gap junctional intercellular communication (Darrow, B. J., et al. (1995). Circ Res 76: 381; Lin R, et al. (2001) J Cell Biol 154(4):815). Antisense technology has been proposed for the modulation of the expression for genes implicated in viral, fungal and metabolic diseases. See, for example, U.S. Pat. No. 5,166,195, (oligonucleotide inhibitors of HIV) and U.S. Pat. No. 5,004,810 (oligomers for hybridizing to herpes simplex virus Vmw65 mRNA and inhibiting replication). See also U.S. Pat. No. 7,098,190 issued to Becker and Green (“Formulations comprising antisense nucleotides to connexins”). Peptide inhibitors of gap junctions and hemichannels have also been reported. See for example Berthoud, V. M. et al., Am J. Physiol. Lung Cell Mol. Physiol. 279:L619-L622 (2000); Evans, W. H. and Boitano, S. Biochem. Soc. Trans. 29:606-612, and De Vriese A. S., et al. Kidney Int. 61:177-185 (2001). See also Becker and Green PCT/US06/04131 (“Anti-connexin compounds and uses thereof”).

Despite advances in the understanding of the principles underlying the wound healing process, there remains a significant unmet need in suitable therapeutic options for wound care, including wounds that do not heal at expected rates, such as delayed-healing wounds, incompletely healing wounds, and chronic wounds. Such therapeutics compositions and treatments are described and claimed herein.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The invention generally relates to the use of anti-osteopontin polynucleotide, alone or in combination with one or more anti-connexin polynucleotides (for example, connexin inhibitors such as alpha-1 connexin oligodeoxynucleotides), which may be optionally combined with one or more anti-connexin peptides, peptidomimetics (for example, alpha-1 anti-connexin peptides or peptidomimetics), gap junction closing or blocking compounds, hemichannel closing or blocking compounds, and connexin carboxy-terminal polypeptides, e.g., polypeptides that bind to a Z0-1 binding site, for the treatment of wounds, including acute, delayed healing and chronic wounds.

Compositions and methods of the invention that employ one or more anti-osteopontin agents for the treatment of acute, delayed healing and chronic wounds are disclosed and claimed.

The anti-osteopontin agents may be combined with one or more anti-connexin agents. For example, compositions and methods of the invention that employ an anti-osteopontin agent in combination with a first anti-connexin agent in combination with a second anti-connexin agent are disclosed and claimed. A first anti-connexin agent may be selected from the group consisting of anti-connexin polynucleotides, anti-connexin peptides, anti-connexin peptidomimetics, gap junction closing or blocking compounds, hemichannel closing or blocking compounds, and connexin carboxy-terminal polypeptides useful for wound healing. A second anti-connexin agent is selected from the above group as modified to subtract the subcategory of anti-connexin agents from which the first anti-connexin agent was selected.

The invention includes a pharmaceutical composition comprising a therapeutically acceptable amount of one or more anti-ostepontin agents in amounts effective to promote healing or tissue repair in a subject. In one embodiment, the composition comprises a therapeutically acceptable amount of one or more anti-ostepontin agents in amounts effective to reduce scaring. In one embodiment, the composition comprises a therapeutically acceptable amount of one or more anti-ostepontin agents in amounts effective to downregulate expression of osteopontin at and/or around a wound site.

The invention includes a pharmaceutical composition comprising a therapeutically acceptable amount of one or more anti-ostepontin agents and one or more anti-connexin polynucleotides.

The invention includes a pharmaceutical composition comprising a therapeutically acceptable amount of one or more anti-ostepontin agents and one or more anti-connexin peptides, anti-connexin peptidomimetics, gap junction closing or blocking compounds, hemichannel closing or blocking compounds, and connexin carboxy-terminal polypeptides useful for wound healing.

The invention includes a pharmaceutical composition comprising an anti-osteopontin agent and a first anti-connexin agent and a second anti-connexin agent, wherein the first anti-connexin agent is selected from the group consisting of anti-connexin polynucleotides, anti-connexin peptides, anti-connexin peptidomimetics, gap junction closing or blocking compounds, hemichannel closing or blocking compounds, and connexin carboxy-terminal polypeptides useful for wound healing, and the second anti-connexin agent is selected from the above group as modified to subtract the subcategory of anti-connexin agents from which the first anti-connexin agent was selected.

Preferred anti-osteopontin agents are anti-osteopontin antisense polynucleotides. Preferred anti-connexin polynucleotides are anti-connexin 43 oligodeoxynucleotides (ODN). Preferred peptides or peptidomimetics, are anti-connexin 43 peptides or peptidomimetics, e.g., anti-connexin 43 hemichannel blocking peptides or anti-connexin 43 hemichannel blocking peptidomimetics. Preferred gap junction closing compounds and hemichannel closing compounds are connexin 43 gap junction closing compounds and connexin 43 hemichannel closing compounds. Preferred connexin carboxy-terminal polypeptides are connexin 43 carboxy-terminal polypeptides. Most preferably, in the case of gap junction closing compound and hemichannel closing compounds useful for wound healing the gap junction or hemichannel is a connexin 43 gap junction or hemichannel. Most preferably, in the case of connexin carboxy-terminal polypeptides useful for wound healing, the connexin is connexin 43.

Such compositions include, for example, topical and inhaled delivery forms and formulations. Such delivery forms and formulations include those for the treatment of a subject as disclosed herein.

Pharmaceutical compositions are also provided in the form of a combined preparation, for example, as an admixture of an anti-osteopontin agent and one or more anti-connexin agents, for example one or more anti-connexin polynucleotides and/or one or more anti-connexin peptides or peptidomimetics.

The term “a combined preparation” includes a “kit of parts” in the sense that the combination partners as defined above can be dosed independently or by use of different fixed combinations with distinguished amounts of the combination partners (a) and (b) and/or (c), i.e. simultaneously, separately or sequentially, where in partner (a) comprises an anti-osteopontin agent, (b) comprises and anti-connexin polynucleotide and (c) comprises an anti-connexin peptide, anti-connexin peptidomimetic, gap junction closing or blocking compound, hemichannel closing or blocking compound, or a connexin carboxy-terminal polypeptide useful for wound healing. The parts of the kit can then, for example, be administered simultaneously or chronologically staggered, that is at different time points and with equal or different time intervals for any part of the kit of parts.

In one embodiment, a combined preparation is adminstered, wherein two or more separate compositions are administered to a subject, wherein the first composition comprises a therapeutically effective amount of an antiosteopontin agent and the second composition comprises a therapeutically effective amount of an anti-connexin 43 polynucleotide, anti-connexin 43 peptide or peptidomimetic. In another embodiment a third composition is administered comprising one or more anti-connexin polynucleotides, peptides, or peptidomimetics. The third composition may also comprise one or more gap junction closing or blocking compounds, hemichannel closing or blocking compounds, or connexin carboxy-terminal polypeptides useful for wound healing.

Pharmaceutical compositions are provided for combined, simultaneous, separate sequential or sustained administration. In one embodiment, a composition comprising one or more anti-osteopontin agents is administered at or about the same time as one or more anti-connexin agents. In one embodiment, a composition comprising one or more anti-osteopontin agents is administered within at least about thirty minutes of one or more anti-connexin agents. In one embodiment, a composition comprising one or more anti-osteopontin agents is administered within at least about 1-2 hours of one or more anti-connexin agents. In one embodiment, a composition comprising one or more anti-osteopontin agents is administered within at least about twelve hours of one or more anti-connexin agents. In one embodiment, a composition comprising one or more anti-osteopontin agents is administered within at least about twenty-four hours of one or more anti-connexin agents. In another embodiment the anti-osteopontin agents and anti-connexin agent are administered within about one hour of each other, within about one day of each other, or within about one week of each other. Other embodiments include administration of one or more anti-osteopontin agents and/or one or more anti-connexin agents, and one or more gap junction closing or blocking compounds useful for wound healing, one or more hemichannel closing or blocking compounds useful for wound healing, and/or one or more connexin carboxy-terminal polypeptides useful for wound healing.

In one aspect, the invention includes pharmaceutical compositions, including topical and inhaled delivery forms and formulations, comprising a pharmaceutically acceptable carrier and therapeutically effective amounts of an anti-osteopontin agent alone or in combination with a first anti-connexin agent and a second anti-connexin agent as described herein, for example, an anti-connexin polynucleotide useful for wound healing and one or more anti-connexin peptides or peptidomimetics useful for wound healing.

Examples of anti-ostepontin agents are anti-ostepontin antisense polynucleotides, including antisense oligodeoxynucleotides (“ODN”) set forth in Table 10. Examples of anti-connexin polynucleotides include anti-connexin oligodeoxynucleotides, including antisense (including modified and unmodified backbone antisense), RNAi, and siRNA. Suitable anti-connexin peptides include binding peptides. Suitable anti-connexin agents include for example, antisense ODNs, peptides and peptidomimetics against connexins 43, 26, 37, 30, and 31.1 and 32. In certain embodiments, suitable compositions include multiple anti-connexin agents in combination, including for example, connexin 43, 26, 30, and 31.1. Preferred anti-connexin agents, including anti-connexin polynucleotides and anti-connexin peptides and peptidomimetics, are directed against connexin 43.

The present invention provides for an increase in the rate, extent and/or quality of wound healing through the use of an anti-osteopontin agent, alone or in combination with one or more anti-connexin agents administered simulataneously, separate, or sequentially. In a preferred embodiment, the combined use of a anti-osteopontin agent in combination with one or more anti-connexin agents as described herein, for example, one or more anti-connexin polynucleotides and/or one or more anti-connexin peptides or peptidomimetics has an additive, synergistic or super-additive effect in the promotion of wound healing. In a preferred embodiment, the administration of a combined preparation will have fewer administration time points and/or increased time intervals between administrations as a result of such combined use. In another preferred embodiment, the combined use of an anti-osteopontin agent in combination with one or more anti-connexin agents as described herein, for example, one or more anti-connexin polynucleotides and/or one or more anti-connexin peptides or peptidomimetics, allows a reduced frequency of administration. In another preferred embodiment, the combined use of a anti-osteopontin agent in combination with one or more anti-connexin agents as described herein, for example, one or more anti-connexin polynucleotides and/or one or more anti-connexin peptides or peptidomimetics, allows the use of reduced doses of such agents compared to the dose or doses that may be effective when the agent is administered alone.

In another aspect, the invention includes methods for administering a therapeutically effective amount of an anti-osteopontin agent, alone or in combination with one or more anti-connexin agents as described herein, for example, one or more anti-connexin polynucleotides and/or one or more anti-connexin peptides or peptidomimetics, formulated in a delayed release preparation, a slow release preparation, an extended release preparation, a controlled release preparation, and/or in a repeat action preparation to a subject with a wound, including chronic wounds and wounds characterized in whole or in part by slow, delayed or incomplete wound healing.

In certain other aspects, the invention also relates to methods of using such compositions to treat subjects suffering from or at risk for various diseases, disorders, and conditions associated with a wound, including acute wounds and wounds that do not heal at expected rates, including delayed healing and chronic wounds.

Treatment of a subject, e.g., for a wound, with one or more pharmaceutical compositions of the invention, e.g. one or more anti-osteopontin agents, alone or in combination with e.g., an anti-connexin ODN and/or a connexin hemichannel blocking agent, e.g., a peptide or peptidomimetic, or a first anti-connexin agent and a second anti-connexin agent, may comprise their simultaneous, separate, sequential or sustained administration.

In yet another aspect, the invention includes methods for treating a subject having or suspected of having or predisposed to, or at risk for, any diseases, disorders and/or conditions characterized in whole or in part by a wound or a tissue in need of repair. Such compositions include, for example, topical and inhaled delivery forms and formulations.

In another aspect, the invention provides method of treatment comprising administering to a subject a pharmaceutical composition of the invention for use in the treatment of a wound, including for example, acute, as well as wounds that do not heal at expected rates, including delayed healing and chronic wounds.

In another aspect, the invention provides a method of treatment comprising administering to a subject in need thereof a composition comprising therapeutically effective amounts of an anti-osteopontin agent, alone or in combination with one or more anti-connexin agents.

In yet another aspect, the invention provides a method of treatment comprising administering to a subject in need thereof a first composition and a second composition, said first composition comprising a therapeutically effective amount of an anti-osteopontin agent and said second composition comprising a therapeutically effective amount of an anti-connexin 43 agents, for example, an anticonnexin polynucleotide, peptide or peptidomimetic. In one embodiment the first composition is administered first. In another embodiment, the second composition comprises one or more gap junction closing or blocking compounds useful for wound healing, one or more hemichannel closing or blocking compounds useful for wound healing, and/or one or more connexin carboxy-terminal polypeptides useful for wound healing. In another embodiment, the second composition is administered first. In a further embodiment, the method further comprises administration of a third composition, wherein the third wound healing composition comprises an anti-connexin polynucleotide, peptide or peptidomimetic. In one embodiment the third composition is administered first.

In one aspect, the invention provides a method for treating acute wounds, comprising administering to a subject in need thereof a therapeutically effective amount of a pharmaceutical composition comprising an anti-osteopontin agent, alone or in combination with one or more anti-connexin agents described herein, for example, one or more anti-connexin polynucleotides, anti-connexin peptides or peptidomimetics. In one embodiment, said method comprises administration of one or more pharmaceutical compositions, the first composition comprising one or more anti-osteopontin agents and, optionally, a second pharmaceutical composition comprising one or more anti-connexin polynucleotides, peptides or peptidomimetics. In one embodiment the first composition is administered first. In another embodiment, the second composition is administered first. In a further embodiment, the method further comprises administration of a third composition, wherein the third wound healing composition comprises an anti-connexin polynucleotide, peptide or peptidomimetic. In one embodiment the third composition is administered first. In one embodiment the third composition is administered first. In one embodiment the pharmaceutical compositions are administered topically.

In one aspect, the invention provides a method for treating chronic wounds, or delayed or slow healing wounds, comprising administering to a subject in need thereof a therapeutically effective amount of a pharmaceutical composition comprising an anti-osteopontin agent, alone or in combination with one or more anti-connexin agents described herein, for example, one or more anti-connexin polynucleotides, anti-connexin peptides or peptidomimetics. In one embodiment, said method comprises administration of one or more pharmaceutical compositions, the first composition comprising one or more anti-osteopontin agents and, optionally, a second pharmaceutical composition comprising one or more anti-connexin polynucleotides, peptides or peptidomimetics. In one embodiment the first composition is administered first. In another embodiment, the second composition is administered first. In a further embodiment, the method further comprises administration of a third composition, wherein the third wound healing composition comprises an anti-connexin polynucleotide, peptide or peptidomimetic. In one embodiment the third composition is administered first. In one embodiment the third composition is administered first. In one embodiment the pharmaceutical compositions are administered topically. In certain embodiments the chronic wound is a diabetic ulcer, a diabetic foot ulcer, a venous ulcer, a venous stasis ulcer, a pressure ulcer, a decubitus ulcer, a vasculitic ulcer, an arterial ulcer, an infectious ulcer, a burn ulcer, a trauma-induced ulcer, an inflammatory ulcer, or an ulceration associated with pyoderma gangrenosum. In one embodiment the subject is diabetic. In one embodiment the subject has a cardiovascular disease or condition.

In a further aspect, the invention provides methods for improving or reducing scar formation in a subject in need thereof, for improving or reducing fibrosis in a subject, and for improving or reducing adhesion formation in a subject, comprising administering to said subject a therapeutically effective amount of a pharmaceutical composition comprising an anti-osteopontin agent, alone or in combination with one or more anti-connexin agents described herein, for example, one or more anti-connexin polynucleotides, anti-connexin peptides or peptidomimetics. In one embodiment, said method comprises administration of one or more pharmaceutical compositions, the first composition comprising one or more anti-osteopontin agents and, optionally, a second pharmaceutical composition comprising one or more anti-connexin polynucleotides, peptides or peptidomimetics. In one embodiment the first composition is administered first. In another embodiment, the second composition is administered first. In a further embodiment, the method further comprises administration of a third composition, wherein the third wound healing composition comprises an anti-connexin polynucleotide, peptide or peptidomimetic. In one embodiment the third composition is administered first. In one embodiment the third composition is administered first. In one embodiment the pharmaceutical compositions are administered topically. In another embodiment the pharmaceutical composition is administered by inhalation.

Preferred methods include the sequential or simultaneous administration one or more anti-osteopontin agents alone or in combination with one or more ant anti-connexin agents described herein, for example, one or more anti-connexin polynucleotides, peptides or peptidomimetics, either or both of which are provided in amounts or doses that are less that those used when the agent or agents are administered alone, i.e., when they are not administered in combination, either physically or in the course of treatment of a wound or other condition to be improved. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In a further aspect, the invention includes transdermal patches, dressings, pads, wraps, matrices and bandages capable of being adhered or otherwise associated with the skin of a subject, said articles being capable of delivering a therapeutically effective amount of an anti-osteopontin agent, alone or in combination with one or more anti-connexin agents as described herein, for example, one or more anti-connexin polynucleotides, peptides or peptidomimetics to a subject.

In another aspect, the invention includes an article of manufacture comprising a vessel containing a therapeutically effective amount of one or more anti-osteopontin agents, alone or in combination with one or more anti-connexin agents described herein, for example, one or more pharmaceutically acceptable anti-osteopontin agent and one or more pharmaceutically acceptable anti-connexin polynucleotides, peptides or peptidomimetics and instructions for use, including use for the treatment of a subject.

The invention includes an article of manufacture comprising packaging material containing one or more dosage forms containing one or more anti-osteopontin agents and one or more anti-connexin agents described herein, for example, one or more anti-osteopontin agents and one or more anti-connexin polynucleotides, peptides or peptidomimetics, wherein the packaging material has a label that indicates that the dosage form can be used for a subject having or suspected of having or predisposed to any of the diseases, disorders and/or conditions described or referenced herein, including diseases, disorders and/or conditions characterized in whole or in part by acute, impaired, delayed or chronic wound healing, by fibrosis, or by adhesions. Such dosage forms include, for example, topical delivery forms and formulations.

The invention includes a formulation comprising one or more anti-osteopontin agents and one or more anti-connexin agents described herein, for example, one or more anti-osteopontin agents and one or more anti-connexin polynucleotides, peptides or peptidomimetics in amounts effective to promote healing or tissue repair in a subject, to prevent or reduce fibrosis in a subject, and to prevent or reduce adhesion formation in a subject. The invention includes a formulation comprising one or more anti-osteopontin agents and one or more anti-connexin agents described herein, for example, one or more anti-osteopontin agents and one or more anti-connexin polynucleotides, peptides or peptidomimetics in amounts effective to promote wound healing in a subject. Such formulations include, for example, topical delivery forms and formulations. Preferred formulations include, for example, a pharmaceutical composition of the invention which is formulated as a foam, spray or gel. In one embodiment, the gel is a polyoxyethylene-polyoxypropylene copolymer-based gel or a carboxymethylcellulose-based gel. In a preferred embodiment, the gel is a pluronic gel.

Preferred formulations include one or more anti-osteopontin agents and one or more anti-connexin agents described herein, for example, one or more anti-osteopontin agents and one or more anti-connexin polynucleotides, peptides or peptidomimetics, either or both of which are provided in amounts or doses that are less that those used when the agent or agents are administered alone, i.e., when they are not administered in combination, either physically or in the course of treatment of a wound. Such lesser amounts of agents administered or provided in combination are typically from about one-twentieth to about one-tenth the amount or amounts when administered alone, and may be about one-eighth the amount, about one-sixth the amount, about one-fifth the amount, about one-fourth the amount, about one-third the amount, and about one-half the amount when administered alone.

The invention includes methods for the use of therapeutically effective amounts of compositions one or more anti-osteopontin agents and one or more anti-connexin agents described herein, for example, one or more anti-osteopontin agents and one or more anti-connexin polynucleotides, peptides or peptidomimetics in the manufacture of a medicament. Such medicaments include, for example, topical delivery forms and formulations. Such medicaments include those for the treatment of a subject as disclosed herein. Such medicaments preferably include the reduced amounts of one or more anti-osteopontin agents and one or more anti-connexin agents described herein, for example, one or more anti-osteopontin agents and one or more anti-connexin polynucleotides, peptides or peptidomimetics, as noted herein.

The invention includes method of preparing a medicament for treating a wound, comprising bringing together and an amount of an anti-connexin agent and an anti-connexin agent as described herein, including, for example, a first composition and a second composition wherein said first composition comprises an effective amount of an anti-osteopontin agent and said second composition comprises an effective amount of an anti-connexin polynucleotide, peptide or peptidomimetic. In other embodiments medicatments comprise a third composition comprising an anti-connexin polynucleotide, an anti-connexin peptide or peptidomimetic, a gap junction closing compound useful for wound healing, a hemichannel closing compound useful for wound healing, and/or a connexin carboxy-terminal polypeptide useful for wound healing.

The invention includes methods for the use of a therapeutically effective amount of one or more anti-osteopontin agents and one or more anti-connexin agents described herein, for example, one or more anti-osteopontin agents and one or more anti-connexin polynucleotides, peptides or peptidomimetics in the manufacture of a dosage form. Such dosage forms include, for example, topical and inhaled delivery forms and formulations. Such dosage forms include those for the treatment of a subject as disclosed herein. Such dosage forms preferably include the reduced amounts of the one or more agents as noted herein.

In another aspect, the invention provides for the use of one or more anti-osteopontin agents and one or more anti-connexin agents described herein, for example, one or more anti-osteopontin agents and one or more anti-connexin polynucleotides, peptides or peptidomimetics, in the manufacture of a pharmaceutical product for the promotion of wound healing, improved and/or reduced scarring, reduced fibrosis, or reduced adhesion formation in a patient in need thereof.

In certain other aspect, the invention provides: (i) a package comprising an anti-ostepontin agent together with instructions for use for the promotion (e.g. decrease in healing time, better wound outcome) of wound healing, (ii) a package comprising one or more anti-osteopontin agents together with instructions for use in combination with one or more anti-connexin agents for the promotion of wound healing; and (iii) a package comprising one or more anti-osteopontin agents alone or in combination with one or more anti-connexin agents, together with instructions for use in the promotion of wound healing for a chronic wound.

In a one embodiment the pharmaceutical product of the invention is provided in combination with a wound dressing or wound healing promoting matrix. Suitably the wound dressing or matrix is provided including the form of a solid substrate with an anti-osteopontin agent alone or in combination with ant anti-connexin agent dispersed on or in the solid substrate.

In yet another embodiment, the invention provides for the use of PDGF receptor inhibitors, for example, for wound treatment and to reduce adhesions and scar formation. PDGF receptor inhibitors include, for example, receptor blockers, receptor antagonists, and monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (e.g., an epitope) that makes contact with a particular antibody or other binding molecule, including antibodies and antibody binding fragments directed against PDGF receptors. PDGF receptor inhibitors may be used alone or in conjunction with one or more anti-osteopontin agents and/or anti-connexin agents.

In another embodiment, the invention provides for the use of PDGF inhibitors, for example, for wound treatment and to reduce adhesions and scar formation. PDGF inhibitors include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (e.g., an epitope) that makes contact with a particular antibody or other binding molecule, including antibodies and antibody binding fragments directed against PDGF itself. PDGF inhibitors may be used alone or in conjunction with one or more anti-osteopontin agents and/or anti-connexin agents.

These and other aspects of the present inventions, which are not limited to or by the information in this Brief Summary, are provided below.

### BRIEF DESCRIPTION OF FIGURES

FIG. 1 demonstrates that antisense oligodeoxynucleotides (AS ODN) against osteopontin (OPN) efficiently decrease OPN protein levels in in vivo murine wounds. FIG. 1(A) shows that immunostaining for OPN (brown) reveals expression by wound connective tissue cells at days 1 and 3, which is resolved by 7 days post-wounding. FIG. 1(B) shows double immunofluorescence of wound tissue at 3 days and reveals OPN expression (red) in both F4/80 positive (green) and negative cells. FIG. 1(C) shows Western blot analysis of OPN in control- and OPN AS ODN-treated wound tissue confirms the temporal profile of the immunostaining in A, and indicates that AS ODN treatment leads to depletion of OPN. FIG. 1(D) shows that band intensities were quantified by densitometric analysis on blots from six independent experiments, and values for the OPN/GAPDH ratio are shown (mean±SEM). FIG. 1(E) shows Cy3-tagged OPN AS ODNs (red) were applied to wounds to assess to what depths and in which tissues OPN was being depleted (evaluated at 6 h). Dotted line indicates the epidermal/connective tissue interface and arrow indicates the epidermal wound edge. Inset shows a low magnification view of the same section. Bars: (A) 50 μm; (B) 10 μm; (E) 100 μm; (E, inset) 200 μm.

FIG. 2 demonstrates skin wound healing is accelerated in OPN AS ODN-treated wounds. FIG. 2(A) shows schematic diagram illustrating the location and dimensions of the full-thickness excisional and incisional wounds made to shaved dorsal skin of adult male ICR mice. Dotted lines indicate the axes of sections. FIG. 2(B) shows macroscopic observation of excisional control- and OPN AS ODN-treated wounds at various time-points post-wounding. FIG. 2(C) shows the proportion of the wound remaining open relative to the initial wound area at each time-point after the injury in control (open bars) versus OPN AS ODN-treated (filled bars) wounds (mean±SEM; n=6). FIG. 2(D) shows schematic to indicate measurements derived from histological sections. FIG. 2(E) shows wound re-epithialisation at 3 and 7 days post-wounding (n=9 for both). FIG. 2(F) shows connective tissue wound width remaining after 3, 7, 10 and 14 days of repair in control-versus OPN AS ODN-treated wounds (Day 3 and 7, n=9; Day 10 and 14, n=6). FIG. 2(G) shows expression of α-smooth muscle actin at 3 and 7 days using western blotting. Images shown are representative of 4 independent experiments. FIG. 2(H) shos tissue from incisional wounds was harvested at 14 days post-wounding, stained with Masson's Trichrome, and the extent (cross-sectional area) of granulation tissue visualized and quantified at the mid-point of the wound (indicated by dotted line). FIG. 2(I) shows the area of granulation tissue was significantly decreased by OPN AS ODN-treatment (filled bars), relative to control (open bars) (Day 3 and 7, n=9; Day 10 and 14, n=6). Bars: (B and H) 1 mm.

FIG. 3 demonstrates altered collagen organization following OPN knock-down in wound sites. FIG. 3(A) shows macroscopic views of scars of control- and OPN AS ODN-treated incisional wounds at 21 days post-injury (wound edge marked with asterisk). Images shown are representative of 8 independent experiments. FIG. 3(B) shows microsirius Red stained sections of control- and OPN AS ODN-treated incisional wounds at 21 days, representative of 5 independent experiments. High magnification details from regions indicated are DIC images. FIG. 3(C) shows transmission electron microscope images of connective tissue from unwounded skin, control- and AS ODN-treated 14 day wounds. Higher magnification insets illustrate differing collagen fibril diameters in these tissues. FIG. 3(D) shows graphic representation of the mean diameter of collagen fibrils (±SEM) from at least 5 fields of view of intact, unwounded skin, control ODN-, and OPN AS ODN-treated wounds; unwounded skin (striped bar, n=200), control ODN (open bar, n=300), OPN AS ODN (filled bar, n=150). Bars: (B) (low magnification) 100 μm, (high magnification) 25 μm; (C) 1 μm; (C, inset) 100 nm.

FIG. 4 demonstrates knock-down of OPN dampens the recruitment of inflammatory cells and increases neovascularisation. FIG. 4(A) shows immunohistochemistry (IHC) for myeloperoxidase (neutrophils) and F4/80 (macrophages), or Toluidine Blue staining (mast cells) reveals significantly fewer leukocytes in the wound granulation tissue of AS ODN-treated versus control wounds. FIG. 4(B) shows the number of neutrophils, macrophages, and mast cells at wound sites were quantified for control- (open bars) and OPN AS ODN-treated wounds (filled bars; mean±SEM; n≧5). FIG. 4(C) show real-time RT-PCR analysis determined the expression of Ccl2, TGFβ1, fibronectin, collagen type1α1, and MMP9 at wound sites, relative to GAPDH, in control ODN (open bars) and OPN AS ODN wounds (filled bars; mean±SEM; n=9). FIG. 4(D) shows that CD31/PECAM IHC reveals blood vessels in control- (left) and OPN AS ODN-treated wounds (right), at 7 days post-injury (original wound margins indicated by arrowheads). FIG. 4(E) shows a graphical representation of the number of vessel lumens in the central wound granulation zone at 7 and 14 days. Bars: (low magnification) 200 μm; (high magnification) 50 μm.

FIG. 5 demonstrates regulation of OPN expression by inflammatory cells. FIG. 5(A) shows a schematic diagram of our in vitro assay to determine which leukocyte(s) and which secreted factor(s) are responsible for induction of OPN expression in fibroblasts. Western blot analyses of Swiss 3T3 fibroblast cells treated for 8 h with FIG. 5(B) showing conditioned media (CM) from various leukocytic cell lineages, or FIG. 5(C) with EGF, PDGF-BB, TGFβ1, TGFβ3, IL-6, or TNF-α to determine which conditions lead to increased OPN expression. FIG. 5(D) shows fibroblasts co-treated with macrophage-CM and Gleevec, FIG. 5(E) PDGF-R neutralising antibodies, or FIG. 5(F) wortmannin. FIG. 5(G) shows Swiss 3T3 fibroblasts stimulated with 20 ng/ml PDGF-BB for the time indicated, and either immediately analysed (left), or rinsed in fresh media, and cultured for the remainder of 8 h before being analysed for OPN expression (right).

FIG. 6 demonstrates deoxyribozyme (Dz) and AS ODN cleavage of OPN mRNA in vitro and in vivo. In vitro transcribed OPN mRNA was incubated with Dzs FIG. 6(A), or control and AS ODNs FIG. 6(B) in vitro and cleavage was visualized. Data are representative of two independent experiments. FIG. 6(C) shows the effective dose of OPN AS ODN (943) to decrease OPN expression in in vivo wounds (vs. control ODNs) was assessed by RT-PCR on RNA extracted from wound sites 6 h after the injury. Data are representative of two independent experiments.

### DETAILED DESCRIPTION

### Definitions

As used herein, a “disorder” is any disorder, disease, or condition that would benefit from an agent that reduces fibrosis. For example, included are diseases, disorders and conditions characterized by excess production of fibrous material, including excess production of fibrous material within the extracellular matrix. Also included are diseases, disorders and conditions characterized by replacement of normal tissue elements by abnormal, non-functional, and/or excessive accumulation of matrix-associated components. Also included are diseases, disorders and conditions characterized by adhesion formation. Also included is any disorder, disease, or condition that would benefit from an agent that promotes wound healing and/or reduces swelling, inflammation, and/or scar formation (including abnormal and excessive scarring, including keloid scars, hypertrophic scars, widespread (stretched) scars, and atrophic (depressed) scars). For example, included are wounds resulting from surgery or trauma, wounds that do not heal at expected rates (such as delayed-healing wounds, incompletely healing wounds, chronic wounds, and dehiscent wounds), and wound associated abnormalities in connection with neuropathic, ischemic, microvascular pathology, pressure over bony area (tailbone (sacral), hip (trochanteric), buttocks (ischial), or heel of the foot), reperfusion injury, and valve reflux etiology and conditions.

As used herein, “subject” refers to any mammal, including humans, domestic and farm animals, and zoo, sports, and pet animals, such as dogs, horses, cats, sheep, pigs, cows, etc. The preferred mammal herein is a human, including adults, children, and the elderly. A subject may also be a bird, including zoo, sports, and pet birds. Preferred sports animals are horses and dogs. Preferred pet animals are dogs and cats.

As used herein, “preventing” means preventing in whole or in part, or ameliorating or controlling, or reducing or halting the production or occurrence of the thing or event to be prevented.

As used herein, a “therapeutically effective amount” or “effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve preventing fibrosis. In another aspect of the present invention, the result will involve the prevention and/or reduction of adhesions. In another aspect of the present invention, the result will involve the prevention and/or reduction of scarring and abnormal scarring, as well as prevention and/or reduction of excessive scar formation and other types of abnormal proliferation of tissue, including keloid scars, hypertrophic scars, widespread scars, and atrophic scars.

According to a further aspect, the result will involve the promotion and/or improvement of wound healing and closure of wounds, in whole or in part, including improvements in rates of healing. Other benefits include reductions in swelling, inflammation and/or scar formation, in whole or in part.

As used herein, the terms “treating” and “treatment” refer to both therapeutic treatment and prophylactic or preventative measures. Those in need of treatment include those already with the disorder as well as those prone to having the disorder or diagnosed with the disorder or those in which the disorder is to be prevented. Thus, by way of example, anti-fibrotic applications of compounds and compositions and formulations of the invention administered prior to the formation of fibrosis or fibrotic tissue are within the invention, as are anti-adhesion applications of compounds and compositions and formulations of the invention administered prior to the formation of an adhesion and anti-scarring applications of compounds and compositions and formulations of the invention administered prior to scar formation including, for example, in a scar reduction surgery or procedure.

As used herein, “anti-connexin agents” are compounds that affect or modulate the activity, expression or formation of a connexin, a connexin hemichannel (connexon), or a gap junction. Anti-connexin agents include, without limitation, antisense compounds (e.g. antisense polynucleotides), RNAi and siRNA compounds, antibodies and binding fragments thereof, and peptides and polypeptides, which include “peptidomimetics,” and peptide analogs. In addition to anti-connexin polynucleotides and anti-connexin peptides, peptidomimetics, or gap junction modifying agents, other anti-connexin agents include gap junction closing compounds (e.g., connexin phosphorylation compounds), hemichannel closing or blocking compounds, and connexin carboxy-terminal polypeptide (which can, e.g., block or disrupt ZO-1 protein interactions with connexins such as connexin 43) Preferred anti-connexin agents are anti-connexin 43 agents, anti-connexin 43 gap junction agents, and anti-connexin 43 hemichannel agents. Exemplary anti-connexin agents are discussed in further detail herein.

The terms “peptidomimetic” and “mimetic” include naturally occurring and synthetic chemical compounds that may have substantially the same structural and functional characteristics of protein regions which they mimic. In the case of connexins, these may mimic, for example, the extracellular loops of opposing connexins involved in connexin-connexin docking and cell-cell channel formation.

“Peptide analogs” refer to the compounds with properties analogous to those of the template peptide and may be non-peptide drugs. “Peptidomimetics” (also known as “mimetic peptides”), which include peptide-based compounds, also include such non-peptide based compounds such as peptide analogs. Peptidomimetics that are structurally similar to therapeutically useful peptides may be used to produce an equivalent or enhanced therapeutic or prophylactic effect. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. The mimetic can be either entirely composed of natural amino acids, or non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. For example, a mimetic composition may be useful as an anti-connexin agent if it is capable of down-regulating biological actions or activities of connexins proteins or hemichannels, such as, for example, preventing the docking of hemichannels to form gap-junction-mediated cell-cell communications, or preventing the opening of hemichannels to expose the cell cytoplasm to the extracellular milieu. Peptidomimetics, mimetic peptides, and connexin modulating peptides, as well as compounds, including connexin phosphorylation compounds and connexin carboxy-terminal polypeptides, encompass those described or referenced herein, as well as those as may be known in the art, whether now known or later developed.

The terms “modulator” and “modulation” of connexin activity, as used herein in its various forms, refers to inhibition in whole or in part of the expression or action or activity of a connexin or connexin hemichannel or connexin gap junction and may function as anti-connexin agents.

In general, the term “protein” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks or months after the other provided that both the one or more anti-connexin polynucleotides and one or more anti-connexin peptides or peptidomimetics are concurrently present in effective amounts. The time delay between administration or administrations of the components will vary depending on the exact nature of the components, the interaction there between, and their respective half-lives.

The term “dressing” refers to a dressing for topical application to a wound and excludes compositions suitable for systemic administration. For example, the one or more anti-connexin polynucleotides and the one or more anti-connexin peptides or peptidomimetics may be dispersed in or on a solid sheet of wound contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In certain embodiments the one or more anti-connexin polynucleotides and/or the one or more anti-connexin peptides or peptidomimetics are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “matrix” includes for example, matrices such as collagen, acellular matrices, crosslinked biological scaffold molecules, tissue-based matrices (including pig-based wound healing matrices), cultured epidermal autografts, cultured epidermal allografts, tissue-engineered skin, collagen and glycosaminoglycan dermal matrices inoculated with autologous fibroblasts and keratinocytes, Alloderm (a nonliving allogeneic acellular dermal matrix with intact basement membrane complex), living skin equivalents (e.g., Dermagraft allogeneic dermal fibroblasts grown on degradable scaffold), TransCyte (an extracellular matrix generated by allogeneic human dermal fibroblasts), Apligraf (a living allogeneic bilayered construct containing keratinocytes, fibroblasts and bovine type I collagen), and OrCel (allogeneic fibroblasts and keratinocytes seeded in opposite sides of bilayered matrix of bovine collagen), animal derived dressings (e.g., Oasis's porcine small intestinal submucosa acellular collagen matrix; and E-Z Derm's acellular xenogeneic collagen matrix), tissue-based bioengineered structural frameworks, biomanufactured bioprostheses, and other implanted or applied structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson et al., Trans Am Sac Artif Intern 1990; 36:340-343) or other biomatrix which have been engineered to reduce the antigenic response to the xenograft material. Other matrix useful in promotion of wound healing may include for example, processed bovine pericardium proteins comprising insoluble collagen and elastin (Courtman et al., J Blamed Mater Res 1994; 28:655-666) and other acellular tissue which may be useful for providing a natural microenvironment for host cell migration to accelerate tissue regeneration (Malone et al., J Vase Surg 1984; 1:181-91). In certain embodiments, the matrix material may be supplemented with one or more anti-connexin polynucleotides and/or the one or more anti-connexin peptides or peptidomimetics for site specific release of such agents.

### Wounds and Wound Classification

Chronic wounds, slow healing wounds and incomplete healing wounds often result in infection and can lead to amputation or death. It has been discovered that use of certain compounds, including those described or referenced herein, may block, inhibit, or alter cell communications, which may promote closure and healing in chronic, slow healing and incomplete healing wounds.

By “wound” is meant an injury to any tissue, including for example, acute, delayed, slow or difficult to heal wounds, and chronic wounds. Examples of wounds may include both open and closed wounds. Wounds include, for example, burns, incisions, excisions, lacerations, abrasions, puncture or penetrating wounds, surgical wounds, contusions, hematomas, crushing injuries, and ulcers. Also included are wounds that do not heal at expected rates.

By a “wound that does not heal at the/an expected rate” is meant an injury to any tissue that does not heal in an expected or typical time frame, including delayed, slow or difficult to heal wounds (including delayed or incompletely healing wounds), and chronic wounds. Examples of wounds that do not heal at the expected rate include diabetic ulcers, diabetic foot ulcers, vascultic ulcers, arterial ulcers, venous ulcers, venous stasis ulcers, pressure ulcers, decubitus ulcers, infectious ulcers, trauma-induced ulcers, burn ulcers, ulcerations associated with pyoderma gangrenosum, and mixed ulcers.

As described herein, a delayed or difficult to heal wound may include, for example, a wound that is characterized at least in part by one or more of 1) a prolonged inflammatory phase, 2) a slow forming extracellular matrix, and 3) a stalled or decreased rate of epithelialization.

In the art, the term “chronic wound” refers generally to a wound that has not healed within three months, but can be wounds that have not healed within one or two months. Chronic skin wounds include, for example, pressure ulcers, diabetic ulcers, venous ulcers, arterial ulcers, inflammatory ulcers, and mixed ulcers. The chronic wound may be an arterial ulcer which can include ulcerations resulting from complete or partial arterial blockage. The chronic wound may be a venous stasis ulcer, which can include ulcerations resulting from a malfunction of the venous valve and the associated vascular disease. The chronic wound may be a trauma-induced ulcer.

As used herein, chronic wound may also include, for example, a wound that is characterized at least in part by 1) a chronic self-perpetuating state of wound inflammation, 2) a deficient and defective wound ECM, 3) poorly responding (senescent) wound cells (e.g. fibroblasts), limited ECM production, 4) failure of re-epithelialization due in part to lack of the necessary ECM orchestration and lack of scaffold for migration.

Chronic wounds may also be characterized, for example, by 1) prolonged inflammation and proteolytic activity, leading to ulcerative lesions, including for example, diabetic, pressure (decubitous), venous, and arterial ulcers; 2) prolonged fibrosis in the wound leading to scarring; 3) progressive deposition of matrix in the affected area, 4) longer repair times, 5) less wound contraction, 6) slower re-epithelialization and 7) increased thickness of granulation tissue.

Exemplary chronic wounds may include “pressure ulcer.” Exemplary pressure ulcers may include all 4 stages of wound classifications based on AHCPR (Agency for Health Care Policy and Research, U.S. Department of Health and Human Services) guidelines, including for example, Stage 1. A stage I pressure ulcer is an observable pressure related alteration of intact skin whose indicators as compared to the adjacent or opposite area on the body may include changes in one or more of the following: skin temperature (warmth or coolness), tissue consistency (firm or boggy feel) and/or sensation (pain, itching). The ulcer appears as a defined area of persistent redness in lightly pigmented skin, whereas in darker skin tones, the ulcer may appear with persistent red, blue, or purple hues. Stage 1 ulceration may include nonblanchable erythema of intact skin and the heralding lesion of skin ulceration. In individuals with darker skin, discoloration of the skin, warmth, edema, induration, or hardness may also be indicators of stage 1 ulceration. Stage 2: stage 2 ulceration may be characterized by partial thickness skin loss involving epidermis, dermis, or both. The ulcer is superficial and presents clinically as an abrasion, blister, or shallow crater. Stage 3: stage 3 ulceration may be characterized by full thickness skin loss involving damage to or necrosis of subcutaneous tissue that may extend down to, but not through, underlying fascia. The ulcer presents clinically as a deep crater with or without undermining of adjacent tissue. Stage 4: stage 4 ulceration may be characterized by full thickness skin loss with extensive destruction, tissue necrosis, or damage to muscle, bone, or supporting structures (e.g., tendon, joint capsule).

Exemplary chronic wounds may include “decubitus ulcers.” Exemplary decubitus ulcer may arise as a result of prolonged and unrelieved pressure over a bony prominence that leads to ischemia. The wound tends to occur in patients who are unable to reposition themselves to off-load weight, such as paralyzed, unconscious, or severely debilitated persons. As defined by the U.S. Department of Health and Human Services, the major preventive measures include identification of high-risk patients; frequent assessment; and prophylactic measures such as scheduled repositioning, appropriate pressure-relief bedding, moisture barriers, and adequate nutritional status. Treatment options may include for example, pressure relief, surgical and enzymatic debridement, moist wound care, and control of the bacterial load. In certain embodiments a method of treating a chronic wound is provided wherein the chronic wound is characterized by decubitus ulcer or ulceration which results from prolonged, unrelieved pressure over a bony prominence that leads to ischemia.

Exemplary chronic wounds may include “arterial ulcers.” Arterial ulcers may include those ulcers characterized by complete or partial arterial blockage, which may lead to tissue necrosis and/or ulceration. Signs of arterial ulcer may include, for example, pulselessness of the extremity; painful ulceration; small, punctate ulcers that are usually well circumscribed; cool or cold skin; delayed capillary return time (briefly push on the end of the toe and release, normal color should return to the toe in about 3 seconds or less); atrophic appearing skin (for example, shiny, thin, dry); and loss of digital and pedal hair. Exemplary chronic wounds may include “venous ulcers.” Exemplary venous ulcers may include the most common type of ulcer affecting the lower extremities and may be characterized by malfunction of the venous valve. The normal vein has valves that prevent the backflow of blood. When these valves become incompetent, the backflow of venous blood causes venous congestion. Hemoglobin from the red blood cells escapes and leaks into the extravascular space, causing the brownish discoloration commonly noted. It has been shown that the transcutaneous oxygen pressure of the skin surrounding a venous ulcer is decreased, suggesting that there are forces obstructing the normal vascularity of the area. Lymphatic drainage and flow also plays a role in these ulcers. The venous ulcer may appear near the medial malleolus and usually occurs in combination with an edematous and indurated lower extremity; it may be shallow, not too painful and may present with a weeping discharge from the affected site.

Exemplary chronic wounds may include “venous stasis ulcers.” Exemplary venous stasis ulcer may be characterized by chronic passive venous congestion of the lower extremities results in local hypoxia. One possible mechanism of pathogenesis of these wounds includes the impediment of oxygen diffusion into the tissue across thick perivascular fibrin cuffs. Another mechanism is that macromolecules leaking into the perivascular tissue trap growth factors needed for the maintenance of skin integrity. Additionally, the flow of large white blood cells slows due to venous congestion, occluding capillaries, becoming activated, and damaging the vascular endothelium to predispose to ulcer formation.

Exemplary chronic wounds may include “diabetic foot ulcers.” Diabetic patients with exemplary diabetic foot ulcer are prone to foot ulcerations due to both neurologic and vascular complications. Peripheral neuropathy can cause altered or complete loss of sensation in the foot and/or leg. Diabetic patients with advanced neuropathy loses all ability for sharp-dull discrimination. Any cuts or trauma to the foot may go completely unnoticed for days or weeks in a patient with neuropathy. A patient with advanced neuropathy can lose the ability to sense a sustained pressure insult and, as a result, tissue ischemia and necrosis may occur leading to for example, plantar ulcerations. Additionally, microfractures in the bones of the foot, if unnoticed and untreated, may result in disfigurement, chronic swelling and additional bony prominences. Microvascular disease is one of the significant complications for diabetics which may also lead to ulcerations.

Exemplary chronic wounds can include “traumatic ulcers.” Formation of exemplary traumatic ulcers may occur as a result of traumatic injuries to the body. These injuries include, for example, compromises to the arterial, venous or lymphatic systems; changes to the bony architecture of the skeleton; loss of tissue layers—epidermis, dermis, subcutaneous soft tissue, muscle or bone; damage to body parts or organs and loss of body parts or organs.

Exemplary chronic wounds can include “burn ulcers” including for example, ulceration that occur as a result of a burn injury, including 1st degree burn (i.e. superficial, reddened area of skin); 2nd degree burn (a blistered injury site which may heal spontaneously after the blister fluid has bee removed); 3rd degree burn (burn through the entire skin and usually require surgical intervention for wound healing); scalding (may occur from scalding hot water, grease or radiator fluid); thermal (may occur from flames, usually deep burns); chemical (may come from acid and alkali, usually deep burns); electrical (either low voltage around a house or high voltage at work); explosion flash (usually superficial injuries); and contact burns (usually deep and may occur from muffler tail pipes, hot irons and stoves).

As used herein, a delayed or difficult to heal wound may include, for example, a wound that is characterized at least in part by 1) a prolonged inflammatory phase, 2) a slow forming extracellular matrix (ECM), and 3) a decreased rate of epithelialization.

As used herein, “fibrotic” diseases, disorders, or conditions include those mentioned herein, and further include acute and chronic, clinical or sub-clinical presentation, in which fibrogenic associated biology or pathology is evident. Fibrotic diseases, disorders, or conditions include diseases, disorders or conditions characterized, in whole or in part, by the excess production of fibrous material, including excess production of fibrotic material within the extracellular matrix, or the replacement of normal tissue elements by abnormal, non-functional, and/or excessive accumulation of matrix-associated components. Fibrotic diseases, disorders, or conditions include, for example, fibrogenic-related biology or pathology characterized by fibrosis.

Exemplary fibrotic diseases, disorders and conditions include, for example, scleroderma (including morphea, generalized morphea, or linear scleroderma), kidney fibrosis (including glomerular sclerosis, renal tubulointerstitial fibrosis, progressive renal disease or diabetic nephropathy), cardiac fibrosis (e.g. myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fascitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis and acute fibrosis.

Fibrotic diseases, disorders and conditions may also include contractures. Contractures, including post-operative contractures, refer to a permanent or long term reduction of range of motion due to tonic spasm or fibrosis, or to loss of normal tissue compliance, motion or equilibrium (e.g., muscle, tendon, ligament, fascia, synovium, joint capsule, other connective tissue, or fat). In general, the condition of contracture may involve a fibrotic response with inflammatory components, both acute and chronic. Some of which may be associated with surgery, including a release procedure. Hereditary contractures such as Dupytren's contracture, Peyronie's disease, and Ledderhose's disease are also included.

Fibrosis can be either chronic or acute. Fibrotic conditions include excessive amounts of fibrous tissue, including excessive amounts of extracellular matrix accumulation within a tissue, forming tissue which causes dysfunction and, potentially, organ failure. Chronic fibrosis includes fibrosis of the major organs, most commonly lung, liver, kidney and/or heart. Acute fibrosis (usually with a sudden and severe onset and of short duration) occurs typically as a common response to various forms of trauma including injuries, ischemic illness (e.g. cardiac scarring following heart attack), environmental pollutants, alcohol and other types of toxins, acute respiratory distress syndrome, radiation and chemotherapy treatments. All tissues damaged by trauma can become fibrotic, particularly if the damage is repeated.

Response to injury has been reported to involve coordinated and temporally regulated patterns of mediators and sequence of cellular events in tissues subsequent to injury. The initial injury is reported to trigger coagulation cascade and an acute local inflammatory response followed by mesenchymal cell recruitment, proliferation and matrix synthesis. Uncontrolled matrix accumulation, often involving aberrant cytokine pathways, can lead to fibrotic conditions or disorders. Progressive fibrosis in vital organs such as the lung, kidney, liver, heart, brain and bone marrow, is both a major cause of illness and death.

### Adhesions

Within other aspects of the invention, methods are provided for treating, reducing the incidence or severity of, and/or preventing or retarding adhesions, surgical adhesions and/or secondary surgical adhesions by administering to a patient an anti-connexin polynucleotide.

Adhesion formation is a complex process in which bodily tissues that are normally separate grow together. For example, post-operative adhesions have been reported to occur in about 60% to 90% of patients undergoing major gynecological surgery. Surgical trauma as a result of tissue (e.g. epithelial, connective, muscle, and nerve tissue) drying, ischemia, thermal injury, infection or the presence of a foreign body, has long been recognized as a stimulus for tissue adhesion formation. These adhesions are a major cause of failed surgical therapy and are the leading cause of bowel obstruction and infertility. Other adhesion-treated complications include chronic pelvic pain, urethral obstruction and voiding dysfunction.

Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, adhesion formation can be inhibited.

Secondary surgical adhesions may also form as a result of a corrective surgical procedure designed to correct and existing adhesion. The procedure may be a release or separation procedure.

A wide variety of animal models may be utilized in order to assess a particular therapeutic composition or treatment regimen for its therapeutic potential. Briefly, peritoneal adhesions have been observed to occur in animals as a result of inflicted severe damage which usually involves two adjacent surfaces. Injuries may be mechanical, due to ischemia or as a result of the introduction of foreign material. Mechanical injuries include crushing of the bowel (Choate et al., Arch. Surg. 88:249-254, 1964) and stripping or scrubbing away the outer layers of bowel wall (Gustaysson et al., Acta Chir. Scand 109:327-333, 1955). Dividing major vessels to loops of the intestine induces ischemia (James et al., J. Path. Bact. 90:279-287, 1965). Foreign material that may be introduced into the area includes talcum (Green et al., Proc. Soc Exp. Biol. Med. 133:544-550, 1970), gauze sponges (Lehman and Boys, Ann. Surg 111:427-435, 1940), toxic chemicals (Chancy, Arch. Surg. 60:1151-1153, 1950), bacteria (Moin et al. Am. J. Med Sci. 250:675-679, 1965) and feces (Jackson, Surgery 44:507-518, 1958).

Presently, typical animal models to evaluate prevention of formation of adhesions include the rabbit uterine horn model which involves the abrasion of the rabbit uterus (Linsky et al., J. Reprod. Med. 32(1): 17-20, 1987), the rabbit uterine horn, devascularization modification model which involves abrasion and devascularization of the uterus (Wiseman et al, J. Invest Surg. 7:527-532, 1994) and the rabbit cecal sidewall model which involves the excision of a patch of parietal peritoneum plus the abrasion of the cecum (Wiseman and Johns, Fertil SteriL Suppl: 25S, 1993). Those and other reported evaluation models are described herein.

### Anti-Connexin Agents

Anti-connexin agents of the invention described herein are capable of modulating or affecting the transport of molecules into and out of cells (e.g., blocking or inhibiting or downregulating). Thus, certain anti-connexin agents described herein modulate cellular communication (e.g., cell to cell). Certain anti-connexin agents are gap junction modulation agents. Certain anti-connexin agents modulate or effect transmission of molecules between the cell cytoplasm and the periplasmic or extracellular space. Such anti-connexin agents are generally targeted to connexins and/or connexin hemichannels (connexons) or to gap junctions themselves. Hemichannels and resulting gap junctions that comprise connexins are independently involved in the release or exchange of small molecules between the cell cytoplasm and an extracellular space or tissue in the case of open hemichannels, and between the cytoplasm of adjoining cell in the case of open gap junctions. Thus, an anti-connexin agents provided herein may directly or indirectly reduce coupling and communication between cells or reduce or block communication (or the transmission of molecules) between a cell and extracellular space or tissue, and the modulation of transport of molecules from a cell into an extracellular space or tissue (or from an extracellular space or tissue into a cell) or between adjoining cells is within the scope of anti-connexin agents and embodiments of the invention. Preferably, the connexin is connexin 43.

Any anti-connexin agent that is capable of eliciting a desired inhibition of the passage (e.g. transport) of molecules through a gap junction or connexin hemichannel may be used in embodiments of the invention. Any anti-connexin agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such anti-connexin agents may modulate the passage of molecules through a gap junction or connexin hemichannel with or without gap junction uncoupling (blocking the transport of molecules through gap junctions). Such compounds include, for example, proteins and polypeptides, polynucleotides, and other organic compounds, and they may, for example block the function or expression of a gap junction or a hemichannel in whole or in part, or downregulate the production of a connexin in whole or in part. Certain gap junction inhibitors are listed in Evans, W. H. and Boitano, S. Biochem. Soc. Trans. 29: 606-612 (2001). Other compounds include connexin phosphorylation compounds that close gap junctions and/or hemichannels, in whole or in part, and connexin carboxy-terminal polypeptides. Preferably, the connexin is connexin 43.

Certain anti-connexin agents provide downregulation of connexin expression (for example, by downregulation of mRNA transcription or translation) or otherwise decrease or inhibit the activity of a connexin protein, a connexin hemichannel or a gap junction. In the case of downregulation, this will have the effect of reducing direct cell-cell communication by gap junctions, or exposure of cell cytoplasm to the extracellular space by hemichannels, at the site at which connexin expression is downregulated. Anti-connexin 43 agents are preferred.

Examples of anti-connexin agents include agents that decrease or inhibit expression or function of connexin mRNA and/or protein or that decrease activity, expression or formation of a connexin, a connexin hemichannel or a gap junction. Anti-connexin agents include anti-connexin polynucleotides, such as antisense polynucleotides and other polynucleotides (such as polynucleotides having siRNA or ribozyme functionalities), as well as antibodies and binding fragments thereof, and peptides and polypeptides, including peptidomimetics and peptide analogs that modulate hemichannel or gap junction activity or function. Anti-connexin 43 agents are preferred.

Anti-Connexin Polynucleotides

Anti-connexin polynucleotides include connexin antisense polynucleotides as well as polynucleotides which have functionalities which enable them to down-regulate connexin expression. Other suitable anti-connexin polynucleotides include RNAi polynucleotides and siRNA polynucleotides. Anti-connexin 43 polynucleotides are preferred.

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss). Methods of synthesizing antibodies and binding fragments as well as peptides and polypeptides, including peptidomimetics and peptide analogs are known to those of skill in the art. See e.g. Lihu Yang et al., Proc. Natl. Acad. Sci. U.S.A., 1; 95(18): 1.0836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA, preferably connexin 43 mRNA. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length, for example. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 8. The connexin is typically an α or β connexin. Preferably the connexin is an α connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted.

Anti-connexin polynucleotides include connexin antisense polynucleotides as well as polynucleotides which have functionalities which enable them to down-regulate connexin expression. Other suitable anti-connexin polynucleotides include RNAi polynucleotides and SiRNA polynucleotides.

In one preferred aspect, the antisense polynucleotides are targeted to the in RNA of one connexin protein only. Most preferably, this connexin protein is connexin 43. In another aspect, connexin protein is connexin 26, 30, 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ.ID.NO: 1) |
| 5′ GTA ATT GCG GCA GGA GGA ATT GTT TCT GTC 3′ | (connexin 43) | (SEQ.ID.NO: 2) |
| 5′ GGC AAG AGA CAC CAA AGA CAC TAC CAG CAT 3′ | (connexin 43) | (SEQ.ID.NO: 3) |
| 5′ TCC TGA GCA ATA CCT AAC GAA CAA ATA 3′ | (connexin 26) | (SEQ.ID.NO: 4) |
| 5′ CAT CTC CTT GGT GCT CAA CC 3′ | (connexin 37) | (SEQ.ID.NO: 5) |
| 5′ CTG AAG TCG ACT TGG CTT GG 3′ | (connexin 37) | (SEQ.ID.NO: 6) |
| 5′ CTC AGA TAG TGG CCA GAA TGC 3′ | (connexin 30) | (SEQ.ID.NO: 7) |
| 5′ TTG TCC AGG TGA CTC CAA GG 3′ | (connexin 30) | (SEQ.ID.NO: 8) |
| 5′ CGT CCG AGC CCA GAA AGA TGA GGT C 3′ | (connexin 31.1) | (SEQ.ID.NO: 9) |
| 5′ AGA GGC GCA CGT GAG ACA C 3′ | (connexin 31.1) | (SEQ.ID.NO: 10) |
| 5′ TGA AGA CAA TGA AGA TGT T 3′ | (connexin 31.1) | (SEQ.ID.NO: 11) |
| 5′ TTT CTT TTC TAT GTG CTG TTG GTG A 3′ | (connexin 32) | (SEQ.ID.NO: 12) |

Suitable polynucleotides for the preparation of the combined polynucleotide compositions described herein include for example, polynucleotides to Connexin Cx43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable:

(SEQ.ID.NO: 1)

GTA ATT GCG GCA AGA AGA ATT GTT TCT GTC;

(SEQ.ID.NO: 2)

GTA ATT GCG GCA GGA GGA ATT GTT TCT GTC;
and

(SEQ.ID.NO: 3)

GGC AAG AGA CAC CAA AGA CAC TAC CAG CAT.

For example, suitable antisense polynucleotides for connexins 26, 31.1 and 32 have the following sequences:

(SEQ.ID.NO: 4)

5′ TCC TGA GCA ATA CCT AAC GAA CAA ATA
(connexin 26);

(SEQ.ID.NO: 9)

5′ CGT CCG AGC CCA GAA AGA TGA GGT C
(connexin 31.1);
and

(SEQ.ID.NO: 12)

5′ TTT CTT TTC TAT GTG CTG TTG GTG A
(connexin 32).

Other connexin antisense polynucleotide sequences useful according to the methods of the present invention include:

(SEQ.ID.NO: 5)

5′ CAT CTC CTT GGT GCT CAA CC 3′ (connexin 37);

(SEQ.ID.NO: 6)

5′ CTG AAG TCG ACT TGG CTT GG 3′ (connexin 37);

(SEQ.ID.NO: 7)

5′ CTC AGA TAG TGG CCA GAA TGC 3′ (connexin 30);

(SEQ.ID.NO: 8)

5′ TTG TCC AGG TGA CTC CAA GG 3′ (connexin 30);

(SEQ.ID.NO: 10)

5′ AGA GGC GCA CGT GAG ACA C 3′ (connexin 31.1);
and

(SEQ.ID.NO: 11)

5′ TGA AGA CAA TGA AGA TGT T 3′ (connexin 31.1).

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

Polynucleotide Homologues

Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Connexin Agents/PDGF Receptor Blockers-Antagonists/Anti-Osteopontin Agents

Binding proteins, including peptides, peptidomimetics, antibodies, antibody fragments, and the like, are also suitable modulators of gap junctions and hemichannels. They are also suitable PGR receptor blockers or antagonists. They are also suitable anti-osteopontin agents.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. J Cell Biol 107: 1817-1824 (1988); Meyer R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). Short peptides corresponding to sequences within the extracellular loops of connexins were said to inhibit intercellular communication. Boitano S. and Evans W. Am J Physiol Lung Cell Mol Physiol 279: L623-L630 (2000). The use of peptides as inhibitors of cell-cell channel formation produced by connexin (Cx) 32 expressed in paired Xenopus oocytes has also been reported. Dahl G, et al., Biophys J 67: 1816-1822 (1994). Berthoud, V. M. and Seul, K. H., summarized some of these results. Am J., Physiol. Lung Cell Mol. Physiol. 279: L619-L622 (2000).

Anti-connexin agents include peptides comprising an amino acid sequence corresponding to a transmembrane region (e.g. 1^{st} to 4^{th}) of a connexin (e.g. connexin 45, 43, 26, 30, 31.1, and 37). Anti-connexin agents may comprise a peptide comprising an amino acid sequence corresponding to a portion of a transmembrane region of a connexin 45. Anti-connexin agents include a peptide having an amino acid sequence that comprises about 5 to 20 contiguous amino acids of SEQ.ID.NO:13, a peptide having an amino acid sequence that comprises about 8 to 15 contiguous amino acids of SEQ.ID.NO:13, or a peptide having an amino acid sequence that comprises about 11 to 13 contiguous amino acids of SEQ.ID.NO:13. Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:13. In certain anti-connexin agents provided herein, the extracellular domains of connexin 45 corresponding to the amino acids at positions 46-75 and 199-228 of SEQ.ID.NO:13 may be used to develop the particular peptide sequences. Certain peptides described herein have an amino acid sequence corresponding to the regions at positions 46-75 and 199-228 of SEQ.ID.NO:13. The peptides need not have an amino acid sequence identical to those portions of SEQ.ID.NO:13, and conservative amino acid changes may be made such that the peptides retain binding activity or functional activity. Alternatively, the peptide may target regions of the connexin protein other than the extracellular domains (e.g. the portions of SEQ.ID.NO:13 not corresponding to positions 46-75 and 199-228).

Also, suitable anti-connexin agents comprise a peptide comprising an amino acid sequence corresponding to a portion of a transmembrane region of a connexin 43. Anti-connexin agents include peptides having an amino acid sequence that comprises about 5 to 20 contiguous amino acids of SEQ.ID.NO:14, peptides having an amino acid sequence that comprises about 8 to 15 contiguous amino acids of SEQ.ID.NO:14, or peptides having an amino acid sequence that comprises about 11 to 13 contiguous amino acids of SEQ.ID.NO:14. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:14. Other anti-connexin agents comprise the extracellular domains of connexin 43 corresponding to the amino acids at positions 37-76 and 178-208 of SEQ.ID.NO:14. Anti-connexin agents include peptides described herein which have an amino acid sequence corresponding to the regions at positions 37-76 and 178-208 of SEQ.ID.NO:14. The peptides need not have an amino acid sequence identical to those portions of SEQ.ID.NO:14, and conservative amino acid changes may be made such that the peptides retain binding activity or functional activity. Alternatively, peptides may target regions of the connexin protein other than the extracellular domains (e.g. the portions of SEQ.ID.NO:14 not corresponding to positions 37-76 and 178-208).

```
Connexin 45 (SEQ ID NO. 13)
Met Ser Trp Ser Phe Leu Thr Arg Leu Leu Glu Glu Ile His Asn His
1 5 10 15
Ser Thr Phe Val Gly Lys Ile Trp Leu Thr Val Leu Ile Val Phe Arg
                        20 25 30
Ile Val Leu Thr Ala Val Gly Gly Glu Ser Ile Tyr Tyr Asp Glu Gln
                35 40 45
Ser Lys Phe Val Cys Asn Thr Glu Gln Pro Gly Cys Glu Asn Val Cys
        50 55 60
Tyr Asp Ala Phe Ala Pro Leu Ser His Val Arg Phe Trp Val Phe Gln
65 70 75 80
Ile Ile Leu Val Ala Thr Pro Ser Val Met Tyr Leu Gly Tyr Ala Ile
                                85 90 95
His Lys Ile Ala Lys Met Glu His Gly Glu Ala Asp Lys Lys Ala Ala
                        100 105 110
Arg Ser Lys Pro Tyr Ala Met Arg Trp Lys Gln His Arg Ala Leu Glu
                115 120 125
Glu Thr Glu Glu Asp Asn Glu Glu Asp Pro Met Met Tyr Pro Glu Met
        130 135 140
Glu Leu Glu Ser Asp Lys Glu Asn Lys Glu Gln Ser Gln Pro Lys Pro
145 150 155 160
Lys His Asp Gly Arg Arg Arg Ile Arg Glu Asp Gly Leu Met Lys Ile
                                165 170 175
Tyr Val Leu Gln Leu Leu Ala Arg Thr Val Phe Glu Val Gly Phe Leu
                        180 185 190
Ile Gly Gln Tyr Phe Leu Tyr Gly Phe Gln Val His Pro Phe Tyr Val
                195 200 205
Cys Ser Arg Leu Pro Cys Pro His Lys Ile Asp Cys Phe Ile Ser Arg
        210 215 220
Pro Thr Glu Lys Thr Ile Phe Leu Leu Ile Met Tyr Gly Val Thr Gly
225 230 235 240
Leu Cys Leu Leu Leu Asn Ile Trp Glu Met Leu His Leu Gly Phe Gly
                                245 250 255
Thr Ile Arg Asp Ser Leu Asn Ser Lys Arg Arg Glu Leu Glu Asp Pro
                        260 265 270
Gly Ala Tyr Asn Tyr Pro Phe Thr Trp Asn Thr Pro Ser Ala Pro Pro
                275 280 285
Gly Tyr Asn Ile Ala Val Lys Pro Asp Gln Ile Gln Tyr Thr Glu Leu
        290 295 300
Ser Asn Ala Lys Ile Ala Tyr Lys Gln Asn Lys Ala Asn Thr Ala Gln
305 310 315 320
Glu Gln Gln Tyr Gly Ser His Glu Glu Asn Leu Pro Ala Asp Leu Glu
                                325 330 335
Ala Leu Gln Arg Glu Ile Arg Met Ala Gln Glu Arg Leu Asp Leu Ala
                        340 345 350
Val Gln Ala Tyr Ser His Gln Asn Asn Pro His Gly Pro Arg Glu Lys
                355 360 365
Lys Ala Lys Val Gly Ser Lys Ala Gly Ser Asn Lys Ser Thr Ala Ser
        370 375 380
Ser Lys Ser Gly Asp Gly Lys Asn Ser Val Trp Ile
385 390 395
Connexin 43 (SEQ ID NO. 14)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Gln Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                        180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

Anti-connexin and anti-PDGF receptor peptides may comprise sequences corresponding to a portion of a connexin extracellular domain or PDGF receptor with conservative amino acid substitutions such that peptides are functionally active anti-connexin or anti-PDGF agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 are shown below in Table 2. M1, 2, 3 and 4 refer to the 1^{st} to 4^{th} transmembrane regions of the connexin 43 protein respectively. E1 and E2 refer to the first and second extracellular loops respectively.

TABLE 2
PEPTIDIC INHIBITORS OF INTERCELLULAR
COMMUNICATION (CX43)

| | | |
|---|---|---|
| FEVAFLLIQWI | M3 & E2 | (SEQ.ID.NO: 15) |
| LLIQWYIGFSL | E2 | (SEQ.ID.NO: 16) |
| SLSAVYTCKRDPCPHQ | E2 | (SEQ.ID.NO: 17) |
| VDCFLSRPTEKT | E2 | (SEQ.ID.NO: 18) |
| SRPTEKTIFII | E2 & M4 | (SEQ.ID.NO: 19) |
| LGTAVESAWGDEQ | M1 & E1 | (SEQ.ID.NO: 20) |
| QSAFRCNTQQPG | E1 | (SEQ.ID.NO: 21) |
| QQPGCENVCYDK | E1 | (SEQ.ID.NO: 22) |
| VCYDKSFPISHVR | E1 | (SEQ.ID.NO: 23) |

Table 3 provides additional exemplary connexin peptides used in inhibiting hemichannel or gap junction function. In other embodiments, conservative amino acid changes are made to the peptides or fragments thereof.

TABLE 3

ADDITIONAL PEPTIDIC INHIBITORS OF INTERCELLULAR COMMUNICATION
(CX32, CX43)

| | | |
|---|---|---|
| | | AA's and |
| Connexin | Location | Sequence |

| | | | |
|---|---|---|---|
| Cx32 | E1 39-77 | AAESVWGDEIKSSFICNTLQPGCNSVCYDHFFPIS | (SEQ.ID.NO: 24) |
| | | HVR | |
| Cx32 | E1 41-52 | ESVWGDEKSSFI | (SEQ.ID.NO: 25) |
| Cx32 | E1 52-63 | ICNTLQPGCNSV | (SEQ.ID.NO: 26) |
| Cx32 | E1 62-73 | SVCYDHFFPISH | (SEQ.ID.NO: 27) |
| Cx32 | E2 64-188 | RLVKCEAFPCPNTVDCFVSRPTEKT | (SEQ.ID.NO: 28) |
| Cx32 | E2 166-177 | VKCEAFPCPNTV | (SEQ.ID.NO: 29) |
| Cx32 | E2 177-188 | VDCFVSRPTEKT | (SEQ.ID.NO: 30) |
| Cx32 | E1 63-75 | VCYDHFFPISHVR | (SEQ.ID.NO: 31) |
| Cx32 | E1 45-59 | VWGDEKSSFICNTLQPGY | (SEQ.ID.NO: 32) |
| Cx32 | E1 46-59 | DEKSSFICNTLQPGY | (SEQ.ID.NO: 33) |
| Cx32 | E2 182-192 | SRPTEKTVFTV | (SEQ.ID.NO: 34) |
| Cx32/Cx43 | E2 182-188/ | SRPTEKT | (SEQ.ID.NO: 35) |
| | 201-207 | | |
| Cx32 | E1 52-63 | ICNTLQPGCNSV | (SEQ.ID.NO: 36) |
| Cx40 | E2 177-192 | FLDTLHVCRRSPCPHP | (SEQ.ID.NO: 37) |
| Cx43 | E2 188-205 | KRDPCHQVDCFLSRPTEK | (SEQ.ID.NO: 38) |

Table 4 provides the extracellular loops for connexin family members which are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 4
EXTRACELLULAR LOOPS FOR VARIOUS CONNEXIN FAMILY MEMBERS

| | | |
|---|---|---|
| E1 | | |
| huCx26 | KEVWGDEQADFVCNTLQPGCKNVCYDHYFPISHIR | (SEQ.ID.NO: 39) |
| huCx30 | QEVWGDEQEDFVCNTLQPGCKNVCYDHFFPVSHIR | (SEQ.ID.NO: 40) |
| huCx30.3 | EEVWDDEQKDFVCNTKQPGCPNVCYDEFFPVSHVR | (SEQ.ID.NO: 41) |
| huCx31 | ERVWGDEQKDFDCNTKQPGCTNVCYDNYFPISNIR | (SEQ.ID.NO: 42) |
| huCx31.1 | ERVWSDDHKDFDCNTRQPGCSNVCFDEFFPVSHVR | (SEQ.ID.NO: 43) |
| huCx32 | ESVWGDEKSSFICNTLQPGCNSVCYDQFFPISHVR | (SEQ.ID.NO: 44) |
| huCx36 | ESVWGDEQSDFECNTAQPGCTNVCYDQAFPISHIR | (SEQ.ID.NO: 45) |
| huCx37 | ESVWGDEQSDFECNTAQPGCTNVCYDQAFPISHIR | (SEQ.ID.NO: 46) |
| huCx40.1 | RPVYQDEQERFVCNTLQPGCANVCYDVFS PVSHLR | (SEQ.ID.NO: 47) |
| huCx43 | ESAWGDEQSAFRCNTQQPGCENVCYDKSFPISHVR | (SEQ.ID.NO: 48) |
| huCx46 | EDVWGDEQSDFTCNTQQPGCBNVCYBRAFPISHIR | (SEQ.ID.NO: 49) |
| huCx46.6 | EAIYSDEQAKFTCNTRQPGCDNVCYDAFAPLSHVR | (SEQ.ID.NO: 50) |
| huCx40 | ESSWGDEQADFRCDTIQPGCQNVCTDQAFPISHIR | (SEQ.ID.NO: 51) |
| huCx45 | GESIYYDEQSKFVCNTEQPGCENVCYDAFAPLSHVR | (SEQ.ID.NO: 52) |
| E2 | | |
| huCx26 | MYVFYVMYDGFSMQRLVKCNAWPCPNTVDCFVSRPTEKT | (SEQ.ID.NO: 53) |
| huCx30 | MYVFYFLYNGYHLPWVLKCGIDPCPNLVDCFISRPTEKT | (SEQ.ID.NO: 54) |
| huCx30.3 | LYIFHRLYKDYDMPRVVACSVEPCPHTVDCYISRPTEKK | (SEQ.ID.NO: 55) |
| huCx31 | LYLLHTLWHGFNMPRLVQCANVAPCPNIVDCYIARPTEKK | (SEQ.ID.NO: 56) |
| huCx31.1 | LYVFHSFYPKYILPPVVKCHADPCPNIVDCFISKPSEKN | (SEQ.ID.NO: 57) |
| huCx32 | MYVFYLLYPGYAMVRLVKCDVYPCPNTVDCFVSRPTEKT | SEQ.ID.NO: 58) |
| huCx36 | LYGWTMEPVFVCQRAPCPYLVDCFVSRPTEKT | (SEQ.ID.NO: 59) |
| huCx37 | LYGWTMEPVFVCQRAPCPYLVDCFVSRPTEKT | (SEQ.ID.NO: 60) |
| huCx40.1 | GALHYFLFGFLAPKKFPCTRPPCTGVVDCYVSRPTSKS | (SEQ.ID.NO: 61) |
| huCx43 | LLIQWYIYGFSLSAVYTCKRDPCPHQVDCFLSRPTEKT | (SEQ.ID.NO: 62) |
| huCx46 | IAGQYFLYGFELKPLYRCDRWPCPNTVDCFISRPTEKT | (SEQ.ID.NO: 63) |
| huCx46.6 | LVGQYLLYGFEVRPFFPCSRQPCPHVVDCFVSRPTEKT | (SEQ.ID.NO: 64) |
| huCx40 | IVGQYFIYGIFLTTLHVCRRSPCPHPVNCYVSRPTEKN | (SEQ.ID.NO: 65) |
| huCx45 | LIGQYFLYGFQVHPFYVCSRLPCHPKIDCFISRPTEKT | (SEQ.ID.NO: 66) |

Table 5 provides the extracellular domain for connexin family members which may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 5
EXTRACELLULAR DOMAINS

| | | |
|---|---|---|
| Peptide | VDCFLSRPTEKT | (SEQ.ID.NO: 18) |
| Peptide | SRPTEKTIFII | (SEQ.ID.NO: 19) |
| huCx43 | LLIQWYIYGFSLSAVYTCKRDPCPHQVDCFLSRPTEKTIFII | (SEQ.ID.NO: 67) |
| huCx26 | MYVFYVMYDGFSMQRLVKCNAWPCPNTVDCFVSRPTEKTVFTV | (SEQ.ID.NO: 68) |
| huCx30 | YVFYFLYNGYHLPWVLKCGIDPCPNLVDCFISRPTEKTVFTI | (SEQ.ID.NO: 69) |
| huCx30.3 | LYIFHRLYKDYDMPRVVACSVEPCPHTVDCYISRPTEKKVFTY | (SEQ.ID.NO: 70) |
| huCx31 | LYLLHTLWHGFNMPRLVQCANVAPCPNIVDCYIARPTEKKTY | (SEQ.ID.NO: 71) |
| huCx31.1 | LYVFHSFYPKYILPPVVKCHADPCPNIVDCFISKPSEKNIFTL | (SEQ.ID.NO: 72) |
| huCx32 | MYVFYLLYPGYAMVRLVKCDVYPCPNTVDCFVSRPTEKTVFTV | (SEQ.ID.NO: 73) |
| huCx36 | LYGWTMEPVFVCQRAPCPYLVDCFVSRPTEKTIFII | (SEQ.ID.NO: 74) |
| huCx37 | LYGWTMEPVFVCQRAPCPYLVDCFVSRPTEKTIFII | (SEQ.ID.NO: 75) |
| huCx40.1 | GALHYFLFGFLAPKKFPCTRPPCTGVVDCYVSRPTEKSLLML | (SEQ.ID.NO: 76) |
| huCx46 | IAGQYFLYGFELKPLYRCDRWPCPNTVDCFISRPTEKTIFII | (SEQ.ID.NO: 77) |
| huCx46.6 | LVGQYLLYGFEVRPFFPCSRQPCPHVVDCFVSRPTEKTVFLL | (SEQ.ID.NO: 78) |
| huCx40 | IVGQYFIYGIFLTTLHVCRRSPCPHPVNCYSRPTEKNVFIV | (SEQ.ID.NO: 79) |
| huCx45 | LIGQYFLYGFQVHPFYVCSRLPCHPKIDCFISRPTEKTIFLL | (SEQ.ID.NO: 80) |

Table 6 provides peptides inhibitors of connexin 40 shown with reference to the extracellular loops (E1 and E2) of connexin 40. The bold amino acids are directed to the transmembrane regions of connexin 40.

TABLE 6
Cx40 peptide inhibitors

| | |
|---|---|
| E2 | |
| LGTAAESSWGDEQADFRCDTIQPGCQNVCTDQAFPISHIRFWVLQ | (SEQ.ID.NO: 81) |
| LGTAAESSWGDEQA | (SEQ.ID.NO: 82) |
| DEQADFRCDTIQP | (SEQ.ID.NO: 83) |
| TIQPGCQNVCTDQ | (SEQ.ID.NO: 84) |
| VCTDQAFPISHIR | (SEQ.ID.NO: 85) |
| AFPISHIRFWVLQ | (SEQ.ID.NO: 86) |
| E2 | |
| MEVGFIVGQYFIYGIFLTTLHVCRRSPCPHPVNCYVSRPTEKNVFIV | (SEQ.ID.NO: 87) |
| MEVGFIVGQYF | (SEQ.ID.NO: 88) |
| IVGQYFIYGIFL | (SEQ.ID.NO: 89) |
| GIFLTTLHVCRRSP | (SEQ.ID.NO: 90) |
| RRSPCPHPVNCY | (SEQ.ID.NO: 91) |
| VNCYVSRPTEKN | (SEQ.ID.NO: 92) |
| SRPTEKNVFIV | (SEQ.ID.NO: 93) |

Table 7 provides peptides inhibitors of connexin 45 shown with reference to the extracellular loops (E1 and E2) of connexin 45. The bold amino acids are directed to the transmembrane regions of connexin 45

TABLE 7
Cx45 peptide inhibitors

| | |
|---|---|
| E1 | |
| LTAVGGESIYYDEQSKFVCNTEQPGCENVCYDAFAPLSHVRFWVFQ | (SEQ.ID.NO: 94) |
| LTAVGGESIYYDEQS | (SEQ.ID.NO: 95) |
| DEQSKFVCNTEQP | (SEQ.ID.NO: 96) |
| TEQPGCENVCYDA | (SEQ.ID.NO: 97) |
| VCYDAFAPLSHVR | (SEQ.ID.NO: 98) |
| APLSHVRFWVFQ | (SEQ.ID.NO: 99) |
| E2 | |
| FEVGFLIGQYFLYGFQVHPFYVCSRLPCHPKIDCFISRPTEKTIFLL | (SEQ.ID.NO: 100) |
| FEVGFLIGQYF | (SEQ.ID.NO: 101) |
| LIGQYFLYGFQV | (SEQ.ID.NO: 102) |
| GFQVHPFYVCSRLP | (SEQ.ID.NO: 103) |
| SRLPCHPKIDCF | (SEQ.ID.NO: 104) |
| IDCFISRPTEKT | (SEQ.ID.NO: 105) |
| SRPTEKTIFLL | (SEQ.ID.NO: 106) |

In certain embodiments, it is preferred that certain peptide inhibitors block hemichannels without disrupting existing gap junctions. While not wishing to be bound to any particular theory or mechanism, it is also believed that certain peptidomimetics (e.g. VCYDKSFPISHVR, (SEQ.ID.NO: 23) block hemichannels without causing uncoupling of gap junctions (See Leybeart et al., Cell Commun. Adhes. 10: 251-257 (2003)), or do so in lower dose amounts. The peptide SRPTEKTIFII (SEQ.ID.NO: 19) may also be used, for example to block hemichannels without uncoupling of gap junctions. The peptide SRGGEKNVFIV (SEQ.ID.NO: 107) may be used that as a control sequence (DeVriese et al., Kidney Internat. 61: 177-185 (2002)). Examples of peptide inhibitors for connexin 45 YVCSRLPCHP (SEQ.ID.NO:108), QVHPFYVCSRL (SEQ.ID.NO:109), FEVGFLIGQYFLY (SEQ.ID.NO:110), GQYFLYGFQVHP (SEQ.ID.NO:111), GFQVHPFYVCSR (SEQ.ID.NO:112), AVGGESIYYDEQ (SEQ.ID.NO.:113), YDEQSKFVCNTE (SEQ.ID.NO:114), NTEQPGCENVCY (SEQ.ID.NO:115), CYDAFAPLSHVR (SEQ.ID.NO:116), FAPLSHVRFWVF (SEQ.ID.NO:117) and LIGQY (SEQ.ID.NO:118), QVHPF (SEQ.ID.NO:119), YVCSR (SEQ.ID.NO:120), SRLPC (SEQ.ID.NO:121), LPCHP (SEQ.ID.NO:122) and GESIY (SEQ.ID.NO:123), YDEQSK (SEQ.ID.NO:124), SKFVCN (SEQ.ID.NO:125), TEQPGCEN (SEQ.ID.NO:126), VCYDAFAP (SEQ.ID.NO:127), LSHVRFWVFQ (SEQ.ID.NO:128) The peptides may only be 3 amino acids in length, including SRL, PCH, LCP, CHP, IYY, SKF, QPC, VCY, APL, HVR, or longer, for example: LIQYFLYGFQVHPF (SEQ.ID.NO:129), VHPFYCSRLPCHP (SEQ.ID.NO:130), VGGESIYYDEQSKFVCNTEQPG (SEQ.ID.NO:131), TEQPGCENVCYDAFAPLSHVRF (SEQ.ID.NO:132), AFAPLSHVRFWVFQ (SEQ.ID.NO: 133).

TABLE 8

Table 8A
Human Connexin 43 from GenBank Accession No. M65188 (SEQ.ID.NO: 134)

| | |
|---|---|
| 1 | ggcttttagc gtgaggaaag taccaaacag cagcggagtt ttaaacttta aatagacagg |
| 61 | tctgagtgcc tgaacttgcc ttttcatttt acttcatcct ccaaggagtt caatcacttg |
| 121 | gcgtgacttc actactttta agcaaaagag tggtgcccag gcaacatggg tgactggagc |
| 181 | gccttaggca aactccttga caaggttcaa gcctactcaa ctgctggagg gaaggtgtgg |
| 241 | ctgtcagtac ttttcatttt ccgaatcctg ctgctgggga cagcggttga gtcagcctgg |
| 301 | ggagatgagc agtctgcctt tcgttgtaac actcagcaac ctggttgtga aaatgtctgc |
| 361 | tatgacaagt ctttcccaat ctctcatgtg cgcttctggg tcctgcagat catatttgtg |
| 421 | tctgtaccca cactcttgta cctggctcat gtgttctatg tgatgcgaaa ggaagagaaa |
| 481 | ctgaacaaga aagaggaaga actcaaggtt gcccaaactg atggtgtcaa tgtggacatg |
| 541 | cacttgaagc agattgagat aaagaagttc aagtacggta ttgaagagca tggtaaggtg |
| 601 | aaaatgcgag gggggttgct gcgaacctac atcatcagta tcctcttcaa gtctatcttt |
| 661 | gaggtggcct tcttgctgat ccagtggtac atctatggat tcagcttgag tgctgtttac |
| 721 | acttgcaaaa gagatccctg cccacatcag gtggactgtt tcctctctcg ccccacggag |
| 781 | aaaaccatct tcatcatctt catgctggtg gtgtccttgg tgtccctggc cttgaatatc |
| 841 | attgaactct tctatgtttt cttcaagggc gttaaggatc gggttaaggg aaagagcgac |
| 901 | ccttaccatg cgaccagtgg tgcgctgagc cctgccaaag actgtgggtc tcaaaaatat |
| 961 | gcttatttca atggctgctc ctcaccaacc gctcccctct cgcctatgtc tcctcctggg |
| 1021 | tacaagctgg ttactggcga cagaaacaat tcttcttgcc gcaattacaa caagcaagca |
| 1081 | agtgagcaaa actgggctaa ttacagtgca gaacaaaatc gaatggggca ggcgggaagc |
| 1141 | accatctcta actcccatgc acagcctttt gatttccccg atgataacca gaattctaaa |
| 1201 | aaactagctg ctggacatga attacagcca ctagccattg tggaccagcg accttcaagc |
| 1261 | agagccagca gtcgtgccag cagcagacct cggcctgatg acctggagat ctag |

Table 8B
Human Connexin 43 (SEQ.ID.NO: 135)

| | |
|---|---|
| 1 | atgggtgactggagcgcctt aggcaaactc cttgacaagg ttcaagccta ctcaactgct |
| 61 | ggagggaaggtgtggctgtc agtacttttc attttccgaatcctgctgct ggggacagcg |
| 121 | gttgagtcagcctggggaga tgagcagtct gcctttcgtt gtaacactca gcaacctggt |
| 181 | tgtgaaaatg tctgctatga caagtctttcccaatctctc atgtgcgctt ctgggtcctg |
| 241 | cagatcatat ttgtgtctgt acccacactcttgtacctgg ctcatgtgttctatgtgatg |
| 301 | cgaaaggaag agaaactgaa caagaaagag gaagaactca aggttgccca aactgatggt |
| 361 | gtcaatgtgg acatgcactt gaagcagatt gagataaagaagttcaagta cggtattgaa |
| 421 | gagcatggta aggtgaaaat gcgagggggg ttgctgcgaa cctacatcat cagtatcctc |
| 481 | ttcaagtcta tctttgaggt ggccttcttg ctgatccagt ggtacatcta tggattcagc |
| 541 | ttgagtgctg tttacacttg caaaagagat ccctgcccac atcaggtgga ctgtttcctc |
| 601 | tctcgcccca cggagaaaac catcttcatc atcttcatgc tggtggtgtc cttggtgtcc |
| 661 | ctggccttga atatcattga actcttctat gttttcttca agggcgttaa ggatcgggtt |
| 721 | aagggaaaga gcgaccctta ccatgcgacc agtggtgcgc tgagccctgc caaagactgt |
| 781 | gggtctcaaa aatatgctta tttcaatggc tgctcctcac caaccgctcc cctctcgcct |
| 841 | atgtctcctc ctgggtacaa gctggttact ggcgacagaa acaattcttc ttgccgcaat |
| 901 | tacaacaagc aagcaagtga gcaaaactgg gctaattaca gtgcagaaca aaatcgaatg |
| 961 | gggcaggcgg gaagcaccat ctctaactcc catgcacagccttttgattt ccccgatgat |
| 1021 | aaccagaatt ctaaaaaactagctgctgga catgaattac agccactagc cattgtggac |
| 1081 | cagcgacctt caagcagagc cagcagtcgtgccagcagca gacctcggcctgatgacctg |
| 1141 | gagatctag |

Gap Junction Modulation Agents

Certain anti-connexin agents described herein are capable of modulation or affecting the transport of molecules into and out of cells (e.g. blocking or inhibiting). Thus certain gap junction modulation agents described herein modulate cellular communication (e.g. cell to cell). Certain gap junction modulation agents modulate or affect transmission of molecules between the cell cytoplasm and the periplasmic or extracellular space. Such agents are generally targeted to hemichannels (also called connexins), which may be independently involved in the exchange of small molecules between the cell cytoplasm and an extracellular space or tissue. Thus, a compound provided herein may directly or indirectly reduce coupling between cells (via gap junctions) or between a cell and an extracellular space or tissue (via hemichannels), and the modulation of transport of molecules from a cell into an extracellular space is within the scope of certain compounds and embodiments of the invention.

Any molecule that is capable of eliciting a desired inhibition of the passage (e.g. transport) of molecules through a gap junction or hemichannel may be used in embodiments of the invention. Compounds that modulate the passage of molecules through a gap junction or hemichannel are also provided in particular embodiments (e.g., those that modulate the passage of molecules from the cytoplasm of a cell into an extracellular space). Such compounds may modulate the passage of molecules through a gap junction or hemichannel with or without gap junction uncoupling. Such compounds include, for example, binding proteins, polypeptides, and other organic compound that can, for example, block the function or activity of a gap junction or a hemichannel in whole or in part.

As used herein, “gap junction modulation agent” may broadly include those agents or compounds that prevent, decrease or modulate, in whole or in part, the activity, function, or formation of a hemichannel or a gap junction. In certain embodiments, a gap junction modulation agent prevents or decreases, in whole or in part, the function of a hemichannel or a gap junction. In certain embodiments, a gap junction modulation agent induces closure, in whole or in part, of a hemichannel or a gap junction. In other embodiments, a gap junction modulation agent blocks, in whole or in part, a hemichannel or a gap junction. In certain embodiments, a gap junction modulation agent decreases or prevents, in whole or in part, the opening of a hemichannel or gap junction. In certain embodiments, said blocking or closure of a gap junction or hemichannel by a gap junction modulation agent can reduce or inhibit extracellular hemichannel communication by preventing or decreasing the flow of small molecules through an open channel to and from an extracellular or periplamic space. Peptidomimetics, and gap junction phosphorylation compounds that block hemichannel and/or gap junction opening are presently preferred.

In certain embodiments, a gap junction modulation agent prevents, decreases or alters the activity or function of a hemichannel or a gap junction. As used herein, modification of the gap junction activity or function may include the closing of gap junctions, closing of hemichannels, and/or passage of molecules or ions through gap junctions and/or hemichannels.

Exemplary gap junction modulation agents may include, without limitation, polypeptides (e.g. peptiditomimetics, antibodies, binding fragments thereof, and synthetic constructs), and other gap junction blocking agents, and gap junction protein phosphorylating agents. Exemplary compounds used for closing gap junctions (e.g. phosphorylating connexin 43 tyrosine residue) have been reported in U.S. Pat. No. 7,153,822 to Jensen et al., U.S. Pat. No. 7,250,397, and assorted patent publications. Exemplary peptides and peptidomimetics are reported in Green et al., WO2006134494. See also Gourdie et al., see WO2006069181, and Tudor et al., see WO2003032964.

As used herein, “gap junction phosphorylating agent” may include those agents or compounds capable of inducing phosphorylation on connexin amino acid residues in order to induce gap junction or hemichannel closure. Gap junction modulation exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B (2001) J. Biol. Chem., Vol. 276, Issue 11, 8544-8549. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Polypeptide compounds, including binding proteins (e.g. antibodies, antibody fragments, and the like), peptides, peptidomimetics, and peptidomimetics, are suitable modulators of gap junctions.

Binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding protein known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, connexin, gap junctions, or associated molecules).

Binding proteins will generally have a desired specificity, including but not limited to binding specificity, and desired affinity. Affinity, for example, may be a Ka of greater than or equal to about 104 M-1, greater than or equal to about 106 M-1, greater than or equal to about 107 M-1, greater than or equal to about 108 M-1. Affinities of even greater than about 108 M-1 are suitable, such as affinities equal to or greater than about 109 M-1, about 1010 M-1, about 1011 M-1, and about 1012 M-1. Affinities of binding proteins according to the present invention can be readily determined using conventional techniques, for example those described by Scatchard et al., (1949) Ann. N.Y. Acad. Sci. 51: 660.

The invention includes use of peptides (including peptidomimetic and peptidomimetics) for modulation of gap junctions and hemichannels. By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. (1988) J Cell Biol 107: 1817-1824; Meyer R. A. (1992) J Cell Biol 119: 179-189.

Peptides or variants thereof, can be synthesized in vitro, e.g., by the solid phase peptide synthetic method or by enzyme-catalyzed peptide synthesis or with the aid of recombinant DNA technology. Solid phase peptide synthetic method is an established and widely used method, which is described in references such as the following: Stewart et al., (1969) Solid Phase Peptide Synthesis, W. H. Freeman Co., San Francisco; Merrifield, (1963) J. Am. Chem. Soc. 85 2149; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

The extracellular domains of a hemichannel contributed by two adjacent cells “dock” with each other to form complete gap junction channels. Reagents that interfere with the interactions of these extracellular domains can impair cell-to-cell communication, or with hemichannel opening to the extracellular environment.

Gap junction modulation agents include peptides comprising an amino acid sequence corresponding to a transmembrane region (e.g. 1st to 4th) of a connexin (e.g. connexin 45, 43, 26, 30, 31.1, and 37). Gap junction modulation agents including a peptide comprising an amino acid sequence corresponding to a portion of a transmembrane region of a connexin 43 are preferred for use in the present inventions.

Gap junction modulation agents may comprise a peptide comprising an amino acid sequence corresponding to a portion of a transmembrane region of a connexin 45. Gap junction modulation agents include a peptide having an amino acid sequence that comprises about 5 to 20 contiguous amino acids of SEQ.ID.NO:13, a peptide having an amino acid sequence that comprises about 8 to 15 contiguous amino acids of SEQ.ID.NO:13, or a peptide having an amino acid sequence that comprises about 11 to 13 contiguous amino acids of SEQ.ID.NO:13. Other embodiments are directed to an gap junction modulation compound that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:13. In certain gap junction modulation compounds provided herein, the extracellular domains of connexin 45 corresponding to the amino acids at positions 46-75 and 199-228 of SEQ.ID.NO:13 may be used to develop the particular peptide sequences. Certain peptides described herein have an amino acid sequence corresponding to the regions at positions 46-75 and 199-228 of SEQ.ID.NO:13. The peptides need not have an amino acid sequence identical to those portions of SEQ.ID.NO:13, and conservative amino acid changes may be made such that the peptides retain binding activity or functional activity. Alternatively, the peptide may target regions of the connexin protein other than the extracellular domains (e.g. the portions of SEQ.ID.NO:13 not corresponding to positions 46-75 and 199-228).

Also, suitable gap junction modulation agents can include a peptide comprising an amino acid sequence corresponding to a portion of a transmembrane region of a connexin 43. Gap junction modulation agents include peptides having an amino acid sequence that comprises about 5 to 20 contiguous amino acids of SEQ.ID.NO:14, peptides having an amino acid sequence that comprises about 8 to 15 contiguous amino acids of SEQ.ID.NO:14, or peptides having an amino acid sequence that comprises about 11 to 13 contiguous amino acids of SEQ.ID.NO:14. Other gap junction modulation agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:14. Other gap junction modulation agents comprise the extracellular domains of connexin 43 corresponding to the amino acids at positions 37-76 and 178-208 of SEQ.ID.NO:14. Gap junction modulation agents include peptides described herein which have an amino acid sequence corresponding to the regions at positions 37-76 and 178-208 of SEQ.ID.NO:14. The peptides need not have an amino acid sequence identical to those portions of SEQ.ID.NO:14, and conservative amino acid changes may be made such that the peptides retain binding activity or functional activity. Alternatively, peptides may target regions of the connexin protein other than the extracellular domains (e.g. the portions of SEQ.ID.NO:14 not corresponding to positions 37-76 and 178-208).

Still other anti-connexin agents include connexin carboxy-terminal polypeptides. See Gourdie et al., WO2006/069181.

Gap Junction Modifying Agents—Other Anti-Connexin Agents

Gap junction modulation agents, include agents that close or block gap junctions and/or hemichannels or otherwise prevent or decrease cell to cell communication via gap junctions or prevent or decrease cell communication to the extracellular environment via hemichannels. They include agents or compounds that prevent, decrease or inhibit, in whole or in part, the activity, function, or formation of a hemichannel or a gap junction.

In certain embodiments, a gap junction modulation agent induces closure, in whole or in part, of a hemichannel or a gap junction. In other embodiments, a gap junction modifying agent blocks, in whole or in part, a hemichannel or a gap junction. In certain embodiments, a gap junction modifying agent decreases or prevents, in whole or in part, the opening of a hemichannel or gap junction.

In certain embodiments, said blocking or closure of a gap junction or hemichannel by a gap junction modifying agent can reduce or inhibit extracellular hemichannel communication by preventing or decreasing the flow of small molecules through an open channel to and from an extracellular or periplasmic space. Gap junction modifying agents used for closing hemichannels or gap junctions (e.g. phosphorylating connexin 43 tyrosine residues) have been reported in U.S. Pat. No. 7,153,822 to Jensen et al., U.S. Pat. No. 7,250,397, and assorted patent publications. See also Gourdie et al., see WO2006069181, with regard to connexin carboxy-terminal polypeptides that are said to, for example, inhibit ZO-1 protein binding. Gourdie et al, WO2006069181 describes use of formulations comprising such peptides.

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B, J. Biol. Chem., Vol. 276, Issue 11, 8544-8549, Mar. 16, 2001. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie et al., WO2006/069181.

In certain another aspect, gap junction modifying agent may include, for example, aliphatic alcohols; octanol; heptanol; anesthetics (e.g., halothane), ethrane, Fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g. arachidonic acid, oleic acid and lipoxygenase metabolites; Fenamates (flufenamic (FFA), niflumic (NFA) and meclofenamic acids (MFA)); Genistein; glycyrrhetinic acid (GA):18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g. acidifying agents; polyunsaturated fatty acids; fatty acid GJIC inhibitors (e.g. oleic and arachidonic acids); quinidine; quinine; all trans-retinoic acid; and tamoxifen.

Anti-Osteopontin Agents

As used herein, anti-osteopontin agents provide downregulation of osteopontin expression (for example, by downregulation of mRNA transcription or translation) or otherwise decrease or inhibit the activity of an osteopontin protein.

Anti-osteopontin agents include anti-osteopontin binding peptides and proteins such as, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (e.g., an epitope) that makes contact with a particular antibody or other binding molecule.

Anti-osteopontin agents include anti-osteopontin polynucleotides such as, for example, an anti-osteopontin deoxyribozyme or an anti-osteopontin antisense oligodeoxynucleotide. In one embodiment, the anti-osteopontin agent is an anti-osteopontin deoxyribozyme. Exemplary deoxyribozymes (Dzs) are described in Table 9. Small letters represent the binding arm and capital letters indicate the catalytic core site (GGCTAGCTACAACGA) (SEQ.ID.NO: 136).

TABLE 9

| | |
|---|---|
| Name | Sequence |

| | |
|---|---|
| OPN Dz (223) | caatctca GGCTAGCTACAACGA ggtcgtag (SEQ.ID.NO: 137) |
| OPN Dz (332) | ctatagga GGCTAGCTACAACGA ctgggtgc (SEQ.ID.NO: 138) |
| OPN Dz (449) | cattggaa GGCTAGCTACAACGA tgcttgga (SEQ.ID.NO: 139) |
| OPN Dz (527) | ccacagaa GGCTAGCTACAACGA cctcgctc (SEQ.ID.NO: 140) |
| OPN Dz (555) | tggtgaga GGCTAGCTACAACGA tcgtcaga (SEQ.ID.NO: 141) |
| OPN Dz (563) | catccgaa GGCTAGCTACAACGA ggtgagat (SEQ.ID.NO: 142) |
| OPN Dz (586) | agcagtga GGCTAGCTACAACGA ggtctcat (SEQ.ID.NO: 143) |
| OPN Dz (596) | cttgtgta GGCTAGCTACAACGA tagcagtg (SEQ.ID.NO: 144) |
| OPN Dz (634) | gacatcga GGCTAGCTACAACGA tgtaggga (SEQ.ID.NO: 145) |
| OPN Dz (640) | gttgggga GGCTAGCTACAACGA atcgactg (SEQ.ID.NO: 146) |
| OPN Dz (656) | agctatca GGCTAGCTACAACGA ctcggccg (SEQ.ID.NO: 147) |
| OPN Dz (659) | ccaagcta GGCTAGCTACAACGA cacctcgg (SEQ.ID.NO: 148) |
| OPN Dz (764) | tagactca GGCTAGCTACAACGA cgctcttc (SEQ.ID.NO: 149) |
| OPN Dz (768) | tccttaga GGCTAGCTACAACGA tcaccgct (SEQ.ID.NO: 150) |
| OPN Dz (902) | ctttggaa GGCTAGCTACAACGA gctcaagt (SEQ.ID.NO: 151) |
| OPN Dz (938) | cgatcaca GGCTAGCTACAACGA ccgactga (SEQ.ID.NO: 152) |
| OPN Dz (943) | actatcga GGCTAGCTACAACGA cacatccg (SEQ.ID.NO: 153) |
| OPN Dz (947) | cttgacta GGCTAGCTACAACGA cgatcaca (SEQ.ID.NO: 154) |
| OPN Dz (950) | ttgcttga GGCTAGCTACAACGA tatcgatc (SEQ.ID.NO: 155) |
| OPN Dz (959) | ctttggaa GGCTAGCTACAACGA ttgcttga (SEQ.ID.NO: 156) |
| OPN Dz (1059) | attcggaa GGCTAGCTACAACGA ttcagata (SEQ.ID.NO: 157) |

In another embodiment anti-osteopontin agents are polynucleotides, for example, antisense oligodeoxynucleotides. Exemplary antisense (AS) oligodeoxynucleotides (ODNs) are described in Table 10.

TABLE 10

| Name | Sequence |
|---|---|
| OPN AS ODN (223) | caatctcatggtcgtag (SEQ.ID.NO: 158) |
| OPN AS ODN (332) | ctataggatctgggtgc (SEQ.ID.NO: 159) |
| OPN AS ODN (449) | cattggaattgcttgga (SEQ.ID.NO: 160) |
| OPN AS ODN (527) | ccacagaatcctcgctc (SEQ.ID.NO: 161) |
| OPN AS ODN (640) | gttggggacatcgactg (SEQ.ID.NO: 162) |
| OPN AS ODN (656) | agctatcacctcggccg (SEQ.ID.NO: 163) |
| OPN AS ODN (943) | actatcgatcacatccg (SEQ.ID.NO: 164) |

### Dosage Forms and Formulations and Administration

A therapeutically effective amount of each of the agents of the invention may be administered simultaneously, separately or sequentially and in any order. The agents may be administered separately or as a fixed combination. When not administered as a fixed combination, preferred methods include the sequential administration of one or more anti-osteopontin agents, alone or in combination with one or more anti-connexin agents (e.g. anti-connexin polynucleotides, peptides or peptidomimetics).

Where an anti-osteopontin agent and anti-connexin agent are administered in combination, either or both are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression. Preferably, the anti-connexin agent or agents is/are anti-connexin 43 agent(s).

The agents of the invention of the may be administered to a subject in need of treatment, such as a subject with any of the diseases or conditions mentioned herein. The condition of the subject can thus be improved. The anti-osteopontin and anti-connexin agents may thus be used in the treatment of the subject's body by therapy. They may be used in the manufacture of a medicament to treat any of the conditions mentioned herein. Thus, in accordance with the invention, there are provided formulations by which cell-cell communication can be downregulated in a transient and site-specific manner.

The anti-osteopontin and anti-connexin agents may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, e.g. at least about 95%, at least about 98% or at least about 99% of the polynucleotide (or other anti-connexin agent) or dry mass of the preparation.

Depending on the intended route of administration, the pharmaceutical products, pharmaceutical compositions, combined preparations and medicaments of the invention may, for example, take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose(CMC)-based formulations, and hydroxypropylmethylcellulose(HPMC)-based formulations. Suitable formulations including pluronic gel, have for example about 10 to about 15 percent, suitably about 12 percent, pluronic gel. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the agent to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulation of the invention.

Other suitable formulations include pluronic gel-based formulations, carboxymethylcellulose(CMC)-based formulations, and hydroxypropylmethylcellulose(HPMC)-based formulations. The composition may be formulated for any desired form of delivery, including topical, instillation, parenteral, intramuscular, subcutaneous, or transdermal administration. Other useful formulations include slow or delayed release preparations.

Transdermal delivery can be carried out by methods known in the art or later discovered, including, for example, methods directed to 1) the use of chemical penetration enhancers or skin enhancers; 2) liposome-mediated delivery; 3) iontophoresis; 4) electroporation; 5) sonophoresis; 6) mechanical (e.g., microporation) devices. Exemplary methods suitable for transdermal delivery of the agents disclosed herein can include, for example, methods directed to enhancing the transport of material across the skin pores by increasing the rate of transport across existing pores or by amplifying the number of available skin pores through the creation of artificial pores.

Transdermal delivery can be carried out by the use of chemical or penetration enhancers, including for example, an pharmaceutically acceptable oil of vegetable, nut, synthetic or animal origin including emu oil, ethoxylated oil, PEG, linoleic acid, ethanol, 1-methanol, and/or agents which delipidize the stratum corneum. Suitable oils include meadowfoam oil, castor oil, jojoba oil, corn oil, sunflower oil, sesame oil, and emu oil, all of which may be optionally ethoxylated. Exemplars include those as described in U.S. Pat. No. 7,291,591, U.S. Pat. No. 5,720,1919, U.S. Pat. No. 7,052,715, U.S. Pat. No. 7,033,998, U.S. Pat. No. 6,946,144; U.S. Pat. No. 6,951,658, U.S. Pat. No. 6,759,056, U.S. Pat. No. 6,720,001, U.S. Pat. No. 6,224,853; U.S. Pat. No. 5,695,779; and U.S. Pat. No. 6,750,291. In addition, transdermal patches can also be adapted for delivery of dry powder or lyophilized drugs, and exemplars include those described in U.S. Pat. No. 5,983,135.

Transdermal delivery can be carried out by liposome mediated delivery methods (e.g., delivery facilitated by application of lipophilic membrane active agents). Suitable exemplars may include those described in U.S. Pat. No. 5,910,306, U.S. Pat. No. 5,718,914, and U.S. Pat. No. 5,064,655.

Transdermal delivery systems can also be employed in conjunction with a wide variety of iontophoresis or electrotransport systems. Illustrative electrotransport drug delivery systems are disclosed in U.S. Pat. Nos. 5,147,296, 5,080,646, 5,169,382 and 5,169,383.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may be achieved in various manners.

Transdermal delivery can be carried out by iontophoretic methods (e.g., delivery facilitated by application of low level electrical field to the skin over time). Suitable exemplars may include those described in U.S. Pat. No. 6,731,987, U.S. Pat. No. 6,391,015, U.S. Pat. No. 6,553,255 B1; U.S. Pat. No. 4,940,456, U.S. Pat. No. 5,681,580 and U.S. Pat. No. 6,248,349.

Also, transdermal delivery can be carried out by electroporation methods (e.g., delivery facilitated by brief application of high voltage pulse to create transient pores in the skin). Suitable exemplars may include U.S. Pat. No. 7,008,637, U.S. Pat. No. 6,706,032, U.S. Pat. No. 6,692,456, U.S. Pat. No. 6,587,705, U.S. Pat. No. 6,512,950, U.S. Pat. No. 6,041,253, U.S. Pat. No. 5,968,006 and U.S. Pat. No. 5,749,847.

Transdermal delivery can be carried out by sonophoresis methods (e.g., delivery facilitated by application of pulses of low frequency ultrasound to increase skin permeability). Suitable exemplars may include U.S. Pat. No. 7,232,431, U.S. Pat. No. 7,004,933, U.S. Pat. No. 6,842,641, U.S. Pat. No. 6,868,286, U.S. Pat. No. 6,712,805, U.S. Pat. No. 6,575,956, U.S. Pat. No. 6,491,657, U.S. Pat. No. 6,487,447, U.S. Pat. No. 623,499, and U.S. Pat. No. 6,190,315.

Transdermal delivery can be carried out by methods comprising the use of mechanical devices and/or creation of artificial micropores or microchannels (e.g., microprojections) by inducing mechanical alterations or disruptions in the structural elements, thermal stability properties, membrane fluidity and integrity of the dermal architecture and substructures. Suitable exemplars may include MicroPor (Altea Therapeutics), MacroFlux (Alza Corporation), as well as those as described in U.S. Pat. No. 6,893,655, U.S. Pat. No. 6,730,318, U.S. RE35,474, U.S. Pat. No. 5,484,604, U.S. Pat. No. 5,362,308, U.S. Pat. No. 5,320,850, and U.S. Pat. No. 5,279,544.

Other suitable formulations are formulations that may be inhaled.

Where the anti-osteopontin or anti-connexin agent is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides. The formulation which is administered may contain such transfection agents. Examples of these agents include cationic agents (for example calcium phosphate and DEAE-dextran) and lipofectants (for example Lipofectam™ and Transfectam™), and surfactants.

Where the anti-osteopontin or anti-connexin agent comprises a polynucleotide, conveniently, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

The effective dose for a given subject or condition preferably lies within the dose that is therapeutically effective for at least 50% of the population, and that exhibits little or no toxicity at this level.

The effective dosage of each of the anti-osteopontin or anti-connexin agents or PDGF receptor blockers or antagonists employed in the methods and compositions of the invention may vary depending on a number of factors including the particular anti-connexin agent or agents employed, the combinational partner (if any), the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

The dose at which an anti-osteopontin or anti-connexin agent or PDGF receptor blocker or antagonist is administered to a patient will depend upon a variety of factors such as the age, weight and general condition of the patient, the condition that is being treated, and the particular anti-connexin agent that is being administered.

A suitable therapeutically effective dose of an anti-osteopontin or anti-connexin agent may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight.

Therapeutically effective doses of anti-osteopontin and anti-connexin agents from about 1 to 100, 100-200, 100- or 200-300, 100- or 200- or 300-400, and 100- or 200- or 300- or 400-500 micrograms are appropriate. Doses from about 1-1000 micrograms are also appropriate. Doses up to 2 milligrams may also be used. Doses are adjusted appropriately when the anti-osteopontin or anti-connexin agent or agents are provided in the form of a dressing, typically upward to maintain the desired total dose administration.

Alternatively, in the case of anti-osteopontin oligonucleotides or anti-connexin proteins or peptides, and in the case of anti-connexin oligonucleotides or anti-connexin peptidomimetics, the dosage of each of the agents in the compositions may be determined by reference to the composition's concentration relative to the size, length, depth, area or volume of the area to which it will be applied. For example, in certain topical applications, dosing of the pharmaceutical compositions may be calculated based on mass (e.g. grams) of or the concentration in a pharmaceutical composition (e.g. μg/ul) per length, depth, area, or volume of the area of application. Useful doses range from about 1 to about 10 micrograms per square centimeter of wound size. Certain doses will be about 1-2, about 1-5, about 2-4, about 5-7, and about 8-10 micrograms per square centimeter of wound size. Other useful doses are greater than about 10 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter.

In certain embodiments, the anti-osteopontin or anti-connexin agent composition may be applied at about 0.01 micromolar (μM) or 0.05 μM to about 200 μM, or up to 300 μM or up to 1000 μM or up to 2000 μM or up to 3200 μM or more final concentration at the treatment site and/or adjacent to the treatment site, and any doses and dose ranges within these dose numbers. Preferably, the antisense polynucleotide composition is applied at about 0.05 μM to about 100 μM final concentration, more preferably, the anti-osteopontin or anti-connexin agent composition is applied at about 1.0 μM to about 50 μM final concentration, and more preferably, the anti-osteopontin or anti-connexin agent composition is applied at about 5-10 μM to about 30-50 μM final concentration. Additionally, the combined anti-connexin agent composition is applied at about 8 μM to about 20 μM final concentration, and alternatively the anti-osteopontin or anti-connexin agent composition is applied at about 10 μM to about 20 μM final concentration, or at about 10 to about 15 μM final concentration. In certain other embodiments, the anti-osteopontin or anti-connexin agent is applied at about 10 μM final concentration. In yet another embodiment, the anti-osteopontin or anti-connexin agent composition is applied at about 1-15 μM final concentration. In other embodiments, the anti-osteopontin or anti-connexin agent is applied at about a 20 μM, 30 μM, 40 μM, 50 μM, 60 μM, 70 μM, 80 μM, 90 μM, 100 μM., 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM or 2000 μM-3000 μM or greater.

Anti-osteopontin and anti-connexin agent dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL. Milliliter doses are also appropriate for larger treatment sites.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one anti-osteopontin or anti-connexin agent is used, the dosage of each anti-osteopontin or anti-connexin agent need not be in the same range as the other. For example, the dosage of one anti-osteopontin or anti-connexin agent may be between about 0.01 mg to about 10 mg per kg body weight, and the dosage of another anti-osteopontin or anti-connexin agent may be between about 0.1 mg to about 1 mg per kg body weight.

All doses and dose ranges referenced herein are applicable, for example, to anti-osteopontin and anti-connexin oligonucleotides. These dose ranges are also applicable, for example, to anti-osteopontin proteins and peptides, and to anti-connexin peptides anti-connexin mimetic peptides and anti-connexin peptidomimetics.

Conveniently, the anti-osteopontin or anti-connexin agent is administered in a sufficient amount to downregulate expression of an ostepontin or connexin protein, or, in the case of an anti-connexin agent, modulate gap junction formation or connexin opening for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-osteopontin or anti-connexin agents in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/μl) per length, depth, area, or volume of the area of application.

As noted herein, the doses of an anti-osteopontin or anti-connexin polynucleotide, peptide or peptidomimetic administered in combination, or other anti-connexin agents administered in combination with either or both, can be adjusted down from the doses administered when given alone.

The combined use of several agents may reduce the required dosage for any individual agent because the onset and duration of effect of the different agents may be complementary. In a preferred embodiment, the combined use of two or more anti-osteopontin or anti-connexin agents has an additive, synergistic or super-additive effect.

In some cases, the combination of one or more anti-osteopontin or anti-connexin polynucleotide and one or more anti-osteopontin or anti-connexin peptides or peptidomimetics, or other anti-osteopontin or anti-connexin agents administered in combination with either or both, have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of one or more anti-osteopontin or anti-connexin polynucleotides and one or more anti-osteopontin or anti-connexin peptides or peptidomimetics, or other anti-osteopontin or anti-connexin agents administered in combination with either or both, is statistically significantly higher than the sum of the decrease in adhesion formation by the individual administration of either of the agents alone. Whether produced by combination administration of one or more anti-osteopontin or anti-connexin polynucleotides and one or more anti-osteopontin or anti-connexin peptides or peptidomimetics, or other anti-connexin agents administered in combination with either or both, is “statistically significantly higher” than the expected additive value of the individual compounds may be determined by a variety of statistical methods as described herein and/or known by one of ordinary skill in the art. The term “synergistic” refers to a type of supra-additive inhibition in which, for example, both an anti-connexin polynucleotide and anti-connexin peptide or peptidomimetic, or other anti-connexin agents administered in combination with either or both, individually have the ability to prevent or decrease adhesion formation, for example. The term “potentiated” refers to type of supra-additive effect in which one of the anti-osteopontin or anti-connexin polynucleotide, anti-osteopontin or anti-connexin peptides or peptidomimetics, or other anti-osteopontin or anti-connexin agents administered in combination with either or both, individually has the increased ability to prevent or decrease adhesion formation, for example.

In general, potentiation may be assessed by determining whether the combination treatment produces a mean decrease, by way of example, in adhesion formation in a treatment group that is statistically significantly supra-additive when compared to the sum of the mean decrease in adhesion formation produced by the individual treatments in their treatment groups respectively. The mean decrease in adhesion formation, for example, may be calculated as the difference between control group and treatment group mean decrease in adhesion formation. The fractional decrease in adhesion formation, for example, “fraction affected” (Fa), may be calculated by dividing the treatment group mean decrease in adhesion formation by control group mean decrease in adhesion formation. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by the p-value. A value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of an anti-osteopontin or anti-connexin agent alone, for example, the one or more agents alone, and the combination of the two at fixed molar ratios. CI values of & lt; 1 indicate synergy, CI-1 indicates an additive effect, and CP1 indicates an antagonistic effect. This analysis may be performed using computer software tools, such as CalcuSyn, Windows Software for Dose Effect Analysis (Biosoft(D, Cambridge UK).

Any method known or later developed in the art for analyzing whether a supra-additive effect exists for a combination therapy is contemplated for use in screening for suitable anti-osteopontin and/or anti-connexin agents for use in combination.

In another preferred embodiment, the combined use of one or more anti-osteopontin or anti-connexin polynucleotides and one or more anti-osteopontin or anti-connexin peptides or peptidomimetics reduces the effective dose of any such agent compared to the effective dose when said agent administered alone. In certain embodiments, the effective dose of the agent when used in combination is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-osteopontin or anti-connexin polynucleotides and one or more anti-osteopontin or anti-connexin peptides or peptidomimetics, or other anti-osteopontin or anti-connexin agents in combination with either or both, reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated. Typically, application will be repeated weekly until, for example, wound healing is promoted, or a repeat application may be made in the event that, for example, wound healing slows or is stalled. Doses may be applied 3-7 days apart, or more. In the case of a chronic wound, for example, repeat applications may be made, for example, weekly, or bi-weekly, or monthly or in other frequency for example if and when, for example, wound healing slows or is stalled. For some indications, such as certain ocular uses, more frequent dosing, up to hourly may employed.

One or more anti-osteopontin and/or anti-connexin polynucleotides and one or more anti-osteopontin and/or anti-connexin peptides or peptidomimetics may be administered by the same or different routes. The various agents of the invention can be administered separately at different times during the course of therapy, or concurrently in divided or single combination forms.

In one aspect of the invention the anti-osteopontin and/or anti-connexin polynucleotide is administered in one composition and the anti-osteopontin and/or anti-connexin peptide or peptidomimetic is administered in a second composition. In one embodiment the first composition comprising one or more anti-osteopontin and/or anti-connexin peptides or peptidomimetics is administered before the second composition comprising one or more anti-osteopontin and/or anti-connexin polynucleotides. In one embodiment the first composition comprising one or more anti-osteopontin and/or anti-connexin peptides or peptidomimetics is administered after the second composition comprising one or more anti-osteopontin and/or anti-connexin polynucleotides. In one embodiment the first composition comprising one or more anti-osteopontin and/or anti-connexin peptides or peptidomimetics is administered before and after the second composition comprising one or more anti-osteopontin and/or anti-connexin polynucleotides. In one embodiment the second composition comprising one or more anti-osteopontin and/or anti-connexin polynucleotides is administered before and after the first composition comprising one or more anti-osteopontin and/or anti-connexin peptides or peptidomimetics. In one embodiment the first composition comprising one or more anti-osteopontin and/or anti-connexin peptides or peptidomimetics is administered about the same time as the second composition comprising one or more anti-osteopontin and/or anti-connexin polynucleotides.

Preferably one or more anti-osteopontin or anti-connexin polynucleotides and one or more anti-osteopontin or anti-connexin peptides or peptidomimetics, or other anti-osteopontin or anti-connexin agents, and/or PDGF receptor blockers or antagonists, administered alone or in combination, are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-osteopontin and/or anti-connexin polynucleotides and one or more anti-osteopontin and/or anti-connexin peptides or peptidomimetics, or other anti-osteopontin and/or anti-connexin agents to be administered alone or in combination, is dispersed in a slow release solid matrix such as a matrix of alginate, collagen, or a synthetic bioabsorbable polymer. Preferably, the solid support composition is sterile or low bio-burden. In one embodiment, a wash solution comprising two or more anti-connexin agents can be used.

The delivery of a formulation of the invention comprising one or more active ingredients, over a period of time, in some instances for about 1-2 hours, about 2-4 hours, about 4-6 hours, about 6-8, or about 24 hours or longer, may be a particular advantage in more severe injuries or conditions.

While the delivery period will be dependent upon both the site at which the downregulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-osteopontin and/or anti-connexin polynucleotides and anti-osteopontin and/or one or more anti-connexin peptides or peptidomimetics, or other anti-osteopontin and/or anti-connexin agents, or a PDGF receptor blacker or antagonist, alone or in combination, in a formulation together with a pharmaceutically acceptable carrier or vehicle, particularly in the form of a formulation for continuous or slow-release administration.

As noted, the one or more agents of the invention may be administered before, during, immediately following wounding, for example, or following a procedure likely or suspected to result in a scar, an adhesion, or fibrosis, for example, or within about 180, about 120, about 90, about 60, or about 30 days, but preferably within about 10, about 9, about 8, about 7, about 6, about 5, about 4, about 3, or about 2 days or less, and most preferably within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute following wounding or following a procedure likely or suspected to result in an adhesion, for example. The one or more agents of the invention may also be administered before and/or during a procedure likely or suspected to result in an adhesion, for example.

The agents and agent combinations of the invention can be administered in any manner which achieves a desired result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site. In a preferred embodiment, 0.5% to 20% anti-connexin agent(s) by weight is loaded into a polymeric carrier (as described in the following examples) and applied to the peritubular (mesenteric) surface as a “paste”, “film”, or “wrap” which releases the drug over a period of time such that the incidence of surgical adhesions is reduced. During endoscopic procedures, the polymer preparation may be applied as a “spray”, via delivery ports in the endoscope, to the mesentery of the abdominal and pelvic organs manipulated during the operation. In a particularly preferred embodiment, the peritubular composition is about 0.1% to about 5% active ingredient by weight. In another preferred embodiment, a polymeric coating containing about 0.1% to about 20% or more of active agent(s) is applied to the surface of the surgical implant (e.g., breast implant, artificial joint, vascular graft, etc.) to prevent encapsulation/inappropriate scarring, for example, in the vicinity of the implant. In yet another preferred embodiment, a polymeric implant containing about 0.01% to about 20% or more of active agent or agents by weight is applied directly to the surgical site (e.g., directly into the sinus cavity, chest cavity, abdominal cavity, or at the operative site during neurosurgery) such that adhesion formation, for example, is prevented or reduced. In one embodiment, one or more active agents can be administered via fluoscopically guided intra-articular injection.

In another embodiment, lavage fluid containing about 1 to about 100 μg/cm² (preferably about 10 to about 50 μg/cm²) of an anti-osteopontin and/or anti-connexin agent(s), would be used at the time of or immediately following surgery and administered during surgery or intraperitoneally, by a physician. In all of the embodiments, other anti-osteopontin and/or anti-connexin agents would be administered at equivalent doses adjusted for potency and tolerability of the agent.

The routes of administration and dosages described herein are intended only as a guide since a skilled physician will determine the optimum route of administration and dosage for any particular patient and condition.

Any of the methods of treating a subject having a disease, disorder or condition referenced or described herein and treating subjects following a surgical procedure may utilize the administration of any of the doses, dosage forms, formulations, and/or compositions herein described.

Dressings and Matrices

In one aspect, one or more active agents are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. One or more active agents may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include, for example, the following with one or more anti-osteopontin agents and/or anti-connexin polynucleotides and one or more anti-osteopontin agents and/or anti-connexin peptides or peptidomimetics (or other active agents to be administered alone or in combination with either or both, such as, for example, PDGF receptor blockers or antagonists):

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-osteopontin agents and/or anti-connexin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption and adhesion. In certain embodiment, the composite dressings are comprised of, for example, multiple layers and incorporate a semi- or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

19) Fillers: suitable filler dressings may include, for example, beads, creams, foams, gels, ointments, pads, pastes, pillows, powders, strands or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

### Wound Treatment

General Aspects

The present invention is directed to pharmaceutical compositions and their methods of use wherein the composition comprises therapeutically effective amounts of one or more anti-osteopontin agents alone or in combination with one or more anti-connexin agents. The compositions are useful in enhancing or promoting healing of wounds, for example, including acute wounds and wounds that do not heal at expected rates, such as chronic wounds and other wounds that may be slow to heal or refractory to conventional wound treatment or wound healing promoting therapies.

Equally, in instances of other tissue damage (particularly wounds) the methods and compositions of the invention are effective in promoting the wound healing process, reducing swelling and inflammation, and in minimizing scar formation. These formations are useful in treating fibrotic diseases, disorders and conditions and in treating, reducing the incidence or severity of or preventing or retarding adhesions, surgical adhesions and/or secondary surgical adhesions. The formulations have clear benefit in the treatment of wounds, whether the result of external trauma (including burns), internal trauma, or surgical intervention, as well as chronic wounds.

Compositions

Accordingly, in one aspect, the invention provides compositions for use in therapeutic wound treatment, which comprises an anti-osteopontin agent. In another aspect, the invention provides compositions for use in therapeutic wound treatment, which comprises an anti-osteopontin agent and at least one anti-connexin agent. In a preferred embodiment, the compositions further comprise a pharmaceutically acceptable carrier or vehicle. In another aspect the invention provides compositions for use in therapeutic wound treatment, which comprises a PDGF receptor blocker or antagonist. In another aspect, the invention provides compositions for use in therapeutic wound treatment, which comprises a PDGF receptor blocker or antagonist together with an anti-osteopontin agent and/or an anti-connexin agent.

In one embodiment, the anti-connexin agent is selected from a group consisting of: an anti-connexin polynucleotide, an anti-connexin peptide or peptidomimetic, a gap junction or hemichannel phosphorylation agent or connexin carboxy-terminal polypeptide for wound treatment.

In one preferred embodiment, the anti-connexin polynucleotide is an antisense polynucleotide. In one preferred form, the composition contains one or more antisense polynucleotides to the mRNA of one connexin protein only. Most preferably, this connexin protein is connexin 43.

In another preferred form, the composition comprises an anti-osteopontin agent, an anti-connexin peptide or peptidomimetic and an antisense polynucleotide to the mRNA of a connexin protein. Most preferably, this connexin is connexin 43.

The compositions may comprise polynucleotides or anti-connexin peptides, or other anti-connexin agents with either or both, that are directed to more than one connexin protein. Preferably, one of the connexin proteins to which polynucleotides or anti-connexin peptides or other anti-connexin agents are directed is connexin 43. Other connexins to which the polynucleotides or anti-connexin peptides or other anti-connexin agents are directed may include, for example, connexins 26, 30, 30.3, 31.1, 32, 36, 37, 40, 40.1, 44.6, 45 and 46. Suitable exemplary polynucleotides (and ODNs) directed to various connexins are set forth in Table 1. Suitable anti-connexin peptides are also provided herein. Suitable gap junction or hemichannel phosphorylation agents and connexin carboxy-terminal polypeptides are known in the art.

Accordingly, in one aspect, the invention provides compositions for use in treating wounds, including chronic and slow or delayed healing wounds.

In another aspect, the invention provides compositions for use in treating fibrosis or fibrotic diseases, disorders or conditions.

In an alternate aspect, the invention provides compositions for use in preventing and/or treating abnormal or excessive scarring and/or excessive tissue proliferation and related disorders and conditions.

In a further aspect, the invention provides compositions and methods for their use in preventing and/or decreasing adhesions, including surgical adhesions.

Kits, Medicaments and Articles of Manufacture

Optionally, one or more anti-osteopontin agents, either alone or in combination with one or more anti-connexin polynucleotides and one or more anti-connexin peptides or peptidomimetics and/or other anti-connexin agents, such as a gap junction or hemichannel phosphorylation agent or connexin carboxy-terminal polypeptide, and/or a PDGF receptor blacker or antagonist, may also be used in the manufacture of the medicament.

In one aspect, the invention provides a kit comprising one or more compositions or formulations described. For example, the kit may include a composition comprising an effective amount of one or more anti-osteopontin agents, either alone or in combination with one or more anti-connexin polynucleotides and one or more anti-connexin peptides or peptidomimetics and/or other anti-connexin agents, such as a gap junction or hemichannel phosphorylation agent or connexin carboxy-terminal polypeptide, and/or a PDGF receptor blocker or antagonist.

Articles of manufacture are also provided, comprising a vessel containing a composition or formulation of the invention as described herein and instructions for use for the treatment of a subject. For example, in another aspect, the invention includes an article of manufacture comprising a vessel containing a therapeutically effective amount of one or more anti-osteopontin agents, either alone or in combination with one or more anti-connexin polynucleotides and one or more anti-connexin peptides or peptidomimetics and/or other anti-connexin agents, such as a gap junction or hemichannel phosphorylation agent or connexin carboxy-terminal polypeptide, and/or a PDGF receptor blocker or antagonist, and instructions for use, including use for the treatment of a subject.

In one aspect, the invention provides for a kit for treating wounds, including chronic and slow or delayed healing wounds. In another aspect, the invention provides a kit for treating fibrosis or fibrotic diseases, disorders or conditions. According to an alternate aspect, the invention provides a kit for preventing and/or treating abnormal or excessive scarring and/or excessive tissue proliferation and conditions comprising one or more of the formulations described. In another aspect, the invention provides a kit for preventing and/or decreasing adhesions comprising one or more compositions or formulations described.

Articles of manufacture are provided for preventing and/or treating wounds, including chronic and slow or delayed healing wounds. In another aspect, Articles of manufacture are provided for preventing and/or treating fibrosis or fibrotic diseases, disorders or conditions. Articles of manufacture are also provided for preventing and/or treating abnormal or excessive scarring and/or excessive tissue proliferation and related disorders and conditions. Additional articles of manufacture are provided for preventing and/or decreasing adhesions as described herein.

Treatment

The compositions and formulations of the invention may be used in conjunction or combination with a composition for promoting the healing of wounds, for example, and can also reduce swelling, inflammation and/or scarring. The compositions and formulations of the invention may also be used in conjunction or combination with a composition for promoting and/or improving the healing of acute or chronic wounds, including slow-healing and delayed healing wounds. In one aspect, the wound will be the result of surgery or trauma or underlying medical condition, e.g., diabetes, peripheral edema, vasculitis, or cardiovascular disease.

In one aspect the invention is directed to a method of promoting or improving wound healing in a subject, comprising administration a therapeutically effective amount of one or more anti-osteopontin agents, either alone or in combination with one or more anti-connexin polynucleotides and/or one or more anti-connexin peptides or peptidomimetics or, optionally, one or more anti-connexin 43 polynucleotides and/or one or more anti-connexin 43 peptides or peptidomimetics other anti-connexin agents, such as a gap junction or hemichannel phosphorylation agent or connexin carboxy-terminal polypeptide. A PDGF receptor blocker or antagonist may also be used alone or in combination with any of the above agents. In certain embodiments, the administration of one or more PDGF receptor blockers or antagonists, and/or one or more anti-osteopontin agents, either alone or in combination with one or more anti-connexin polynucleotides, peptides or peptidomimetics, or, optionally, one or more anti-connexin 43 polynucleotides and/or one or more anti-connexin 43 peptides or peptidomimetics other anti-connexin agents, such as a gap junction or hemichannel phosphorylation agent or connexin carboxy-terminal polypeptide, is effective to reduce inflammation, promote cell migration to accelerate wound closure and healing, and/or to facilitate epithelial growth and surface recovery. In certain embodiments, the administration of one or more compositions of the invention is effective to reduce or prevent scar formation, including abnormal scar formation.

In one aspect the invention is directed to a method of promoting or improving wound healing in a subject, comprising administration of one or more anti-osteopontin agents in combination with one or more anti-connexin polynucleotides and/or one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, in an amount effective to regulate epithelial basal cell division and growth. In one embodiment, the anti-connexin agent is a connexin antisense polynucleotide effective to regulate epithelial basal cell division and growth or to regulate outer layer keratin secretion. In one embodiment, the connexin antisense polynucleotide is a connexin 26 antisense polynucleotide, peptide or peptidomimetic, a connexin 43 antisense polynucleotide, peptide, or peptidomimetic or a mixture thereof.

In yet a further aspect, the invention provides a method of decreasing scar formation and/or improving scar appearance in a patient who has suffered a wound, e.g., a surgical wound (such as in, for example, cosmetic, scar revision, and other surgeries), which comprises the step of administering one or more anti-osteopontin agents, either alone or in combination with one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to said wound to downregulate expression of one or more connexin protein(s) at and immediately adjacent the site of said wound. PDGF receptor inhibitors may also be used, alone or in conjunction with one or more anti-osteopontin agents and/or anti-connexin agents. Again, the wound may be the result of trauma or surgery, for example, with the formulation being applied to the wound immediately prior to surgical repair and/or closure thereof. As noted herein, in methods to reduce or improve scar formation or appearance, the anti-connexin peptide or peptidomimetic is preferably administered in combination with, or after or prior to, administration of a suitable amount anti-connexin polynucleotide.

In one aspect the invention is directed to a method of reducing, preventing or ameliorating tissue damage in a subject, comprising administration of one or more anti-osteopontin agents, either alone or in combination with one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide.

In a further aspect, the invention is directed to a method of reducing swelling and/or inflammation as part of treating an acute or chronic wound and/or tissue subjected to physical trauma which comprises the step of administering one or more anti-osteopontin agents, either alone or in combination with one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to or proximate to said wound or tissue. In one embodiment the wound is the result of physical trauma to tissue, including neuronal tissue such as the brain, spinal cord or optic nerve, or skin or eye.

In one aspect the invention is directed to sustained administration of one or more anti-osteopontin agents, either alone or in combination with one or more anti-connexin polynucleotides, peptides or peptidomimetics, or, optionally, to sustained administration of one or more anti-osteopontin agents, either alone or in combination with one or more anti-connexin polynucleotides and/or one or more anti-connexin peptides or peptidomimetics other anti-connexin agents, such as a gap junction or hemichannel phosphorylation agent or connexin carboxy-terminal polypeptide. In one embodiment, the agent or agents are administered for at least at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours. In one embodiment, osteopontin and/or connexin expression is downregulated over a sustained period of time. In another embodiment, osteopontin proteins are bound or connexin hemichannels are blocked or closed, in whole or in part, over a preferred period of time. Preferably, for example, osteopontin and/or connexin 43 expression is downregulated and connexin hemichannel opening is blocked or inhibited, in whole or in part, for a sustained period of time. Conveniently, osteopontin and/or connexin 43 expression is downregulated or hemichannels blocked or inhibited for at least about 1, 2, 4, 6, 8, 10, 12, or 24 hours. According to one embodiment, the wound is a chronic wound. Suitable subjects include a diabetic subject. Other subjects include, for example, those with peripheral edema, vasculitis, or cardiovascular disease.

In one aspect, the present invention provides a method of treating a subject having a wound which comprises sustained administration of an effective amount of one or more PDGF receptor blockers or antagonists and/or one or more anti-osteopontin agents, either alone or in combination with one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to the wound.

In another aspect, methods for treating a subject having a chronic wound are provided. Such methods include administering to the subject one or more anti-osteopontin agents, either alone or in combination with a PDGF receptor blocker or antagonist and/or an anti-connexin agent capable of inhibiting the expression, formation, or activity of a connexin, or a connexin hemichannel.

In one aspect the invention is directed to a method for treatment or prophylaxis of a chronic wound comprising administering to a subject in need thereof an effective amount of an anti-osteopontin agent administered to said chronic wound or a tissue associated with said chronic wound in combination with an anti-connexin agent and/or a PDGF receptor blocker or antagonist. In one embodiment the anti-osteopontin agent is administered in combination with an anti-connexin polynucleotide, preferably a connexin 43 antisense polynucleotide. In another embodiment, the anti-osteopontin agent is administered in combination with an anti-connexin peptide or peptidomimetic, preferably an anti-connexin 43 peptide or peptidomimetic. In another embodiment, the anti-osteopontin agent is administered in combination with an anti-connexin peptide or peptidomimetic and an anti-connexin polypeptide. In another embodiment, the chronic wound is a chronic skin wound and a composition of the present invention is administered to the skin or a tissue associated with the skin of said subject for an effective period of time. A chronic skin wound suitable for treatment may, for example, be selected from the group consisting of pressure ulcers, diabetic ulcers, venous ulcers, arterial ulcers, vasculitic ulcers, and mixed ulcers, and other noted herein. The chronic wound may be an arterial ulcer, which comprises ulcerations resulting from complete or partial arterial blockage. The chronic wound may be a venous stasis ulcer, which comprises ulcerations resulting from a malfunction of the venous valve and the associated vascular disease. The chronic wound may be a trauma-induced ulcer.

When not administered as a fixed combination, preferred methods include the sequential administration of one or more anti-osteopontin agents and one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression. Preferably, the anti-connexin agent or agents is/are anti-connexin 43 agent(s).

In another embodiment for treatment of wounds, including chronic wounds, either or all of the one or more anti-osteopontin agents, one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In one embodiment the method for treatment or prophylaxis of a chronic wound comprises sustained administration of one or more anti-osteopontin agents alone or in combination with one or more anti-connexin polynucleotides, peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide. In one embodiment, the composition or compositions are administered in a sustained release formulation. In another embodiment, the composition or compositions are administered for a sustained period of time. Conveniently, the composition is effective to decrease connexin 43 levels, or block or reduce connexin 43 hemichannel opening, for at least about 1-2 hours, about 2-4 hours, about 4-6 hours, about 4-8 hours, about 12 hours, about 18 hours, or about 24 hours. Subjects which may be treated include diabetic subjects, and patients with other ulcers, including venous ulcers and others described herein and known in the art.

In one aspect the invention is directed to a method of preventing and/or treating fibrosis or fibrotic diseases, disorders or conditions in a subject, comprising administration a therapeutically effective amount of one or more anti-osteopontin agents alone or in combination with one or more anti-connexin polynucleotides and one or more anti-connexin peptides, peptidomimetics, or gap junction modifying agents, such as a gap junction or hemichannel phosphorylation agent or connexin carboxy-terminal polypeptide. In certain embodiments, the administration is effective to reduce fibrosis. In certain embodiments, the administration is effective to prevent or reduce contracture.

In one aspect the invention is directed to a method of preventing and/or treating fibrosis or fibrotic diseases, disorders or conditions in a subject, comprising administration of a therapeutically effective amount of one or more anti-osteopontin optionally with one or more anti-connexin agents effective to reduce fibrosis. In one embodiment, administration of the anti-osteopontin and optional one or more anti-connexin agents is effective to prevent or reduce contracture.

According to one embodiment of the method, the subject has a disorder selected from the group consisting of scleroderma, kidney fibrosis (including diabetic nephropathy), cardiac fibrosis (e.g. myocardial fibrosis), pulmonary fibrosis (e.g., glomerulosclerosis pulmonary fibrosis, idiopathic pulmonary fibrosis, silicosis, asbestosis, interstitial lung disease and fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fascitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis and acute fibrosis. According to this embodiment, the scleroderma may be morphea, generalized morphea, or linear scleroderma. Also according to this embodiment, the kidney fibrosis may be glomerular sclerosis, renal tubulointerstitial fibrosis or progressive renal disease. Further to this embodiment, the pulmonary fibrosis may be diffuse interstitial pulmonary fibrosis.

According to another embodiment of the method, the fibrosis is acute fibrosis. The acute fibrosis may be in response to various forms of trauma including accidental injuries, infections, radiation or chemotherapy treatments.

According to another embodiment of the method, the fibrosis is chronic fibrosis. The invention also includes methods for treating and/or preventing, in whole or in part, various diseases, disorders and conditions, including, for example, capsular contracture, Dupytren's contracture, Volkmann's contracture, Ledderhose's contracture, Peyronie's contracture or recurrence thereof, comprising administering a effective amount of a composition comprising an anti-connexin polynucleotide. In on embodiment, the composition is administered to the site of the injury before, at the time of and/or after a release procedure (e.g., forced manipulation, open release, arthroscopic release, or debulking of scar) to prevent the recurrence of scarred and abnormal tissue and/or further contracture.

In one aspect the invention is directed to a method of for preventing and/or treating abnormal or excessive scarring and/or excessive tissue proliferation and related disorders and conditions in a subject, comprising administration a therapeutically effective amount of one or more one or more anti-osteopontin agents alone or in combination with one or more anti-connexin polynucleotides and/or one or more anti-connexin peptides, peptidomimetics, or gap junction modifying agents, such as a gap junction or hemichannel phosphorylation agent or connexin carboxy-terminal polypeptide. In certain embodiments, the administration is effective to reduce abnormal or excessive scarring and/or excessive tissue proliferation and related disorders and conditions.

In one aspect the invention is directed to a method of for preventing and/or treating abnormal or excessive scarring and/or excessive tissue proliferation and related disorders and conditions in a subject, comprising administration a therapeutically effective amount of an anti-osteopontin agent. In one embodiment, the anti-osteopontin agent is effective to reduce abnormal or excessive scarring and/or excessive tissue proliferation and related disorders and conditions.

In one aspect the invention is directed to sustained administration of an anti-osteopontin agent alone or in combination with one or more anti-connexin agents.

According to one embodiment, the subject has an abnormal scar selected from the group consisting of keloid scars, hypertrophic scars, widespread scars, and atrophic scars.

According to another embodiment, the subjects to be treated include those having experienced trauma, surgical intervention, burns, and other types of injuries that lead, or can lead, to abnormal or excessive scarring, as well as excessive scar formation and other types of abnormal proliferation of tissue, including keloid scars, hypertrophic scars, widespread scars, and atrophic scars.

In certain embodiments, the anti-osteopontin agent and/or anti-connexin agent is administered to epithelial, connective, muscle, and nerve tissue or other tissue exposed or wounded during surgery or as a result of trauma. In one embodiment, the anti-osteopontin agent and/or anti-connexin agent is administered topically. In other embodiments, the anti-osteopontin agent and/or anti-connexin agent is implanted or instilled or injected.

The following examples which will be understood to be provided by way of illustration only and not to constitute a limitation on the scope of the invention.

### EXAMPLES

### Example 1

The following materials and methods were used throughout the examples described herein.

### Wound Model and ODN Treatment

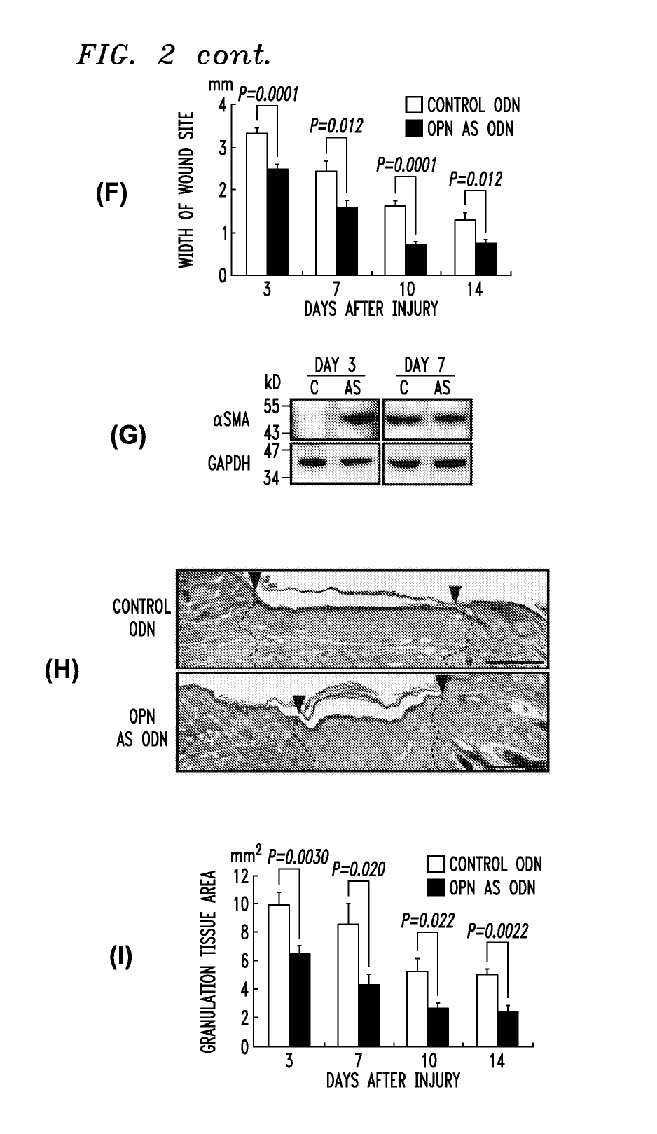

All experiments were conducted according to UK Home Office regulations. Mice (ICR age-matched males; 7-11 wks) were halothane-anaesthetized and 4 full-thickness excisional wounds (4 mm biopsy punch; Kai Industries) or two full-thickness incisional wounds of 1 cm were aseptically made to the shaved dorsal skin (FIG. 2 A). ODNs (control or OPN AS) were topically applied by pipette into the wound cavity immediately post-wounding [50 μl; 1, 2, or 5 μM in 30% Pluronic F-127 gel (see Supp. Materials and Methods); Sigma-Aldrich]. Wounds were recorded using a SteREO Lumar.V12 microscope (Carl Zeiss), and areas calculated using Openlab™ 4.0.2 (Improvision) software. Wound tissue was harvested with a 6 mm biopsy punch.

### Sequences of Control and Antisense (AS) Oligodeoxynucleotides (ODNs).

| Name | Sequence |
|---|---|
| OPN control ODN (223) | ctacgaccatgagattg |
| OPN AS ODN (223) | caatctcatggtcgtag |
| OPN control ODN (332) | gcacccagatcctatag |
| OPN AS ODN (332) | ctataggatctgggtgc |
| OPN control ODN (449) | tccaagcaattccaatg |
| OPN AS ODN (449) | cattggaattgcttgga |
| OPN control ODN (527) | gagcgaggattctgtgg |
| OPN AS ODN (527) | ccacagaatcctcgctc |
| OPN control ODN (640) | cagtcgatgtccccaac |
| OPN AS ODN (640) | gttggggacatcgactg |
| OPN control ODN (656) | cggccgaggtgatagct |
| OPN AS ODN (656) | agctatcacctcggccg |
| OPN control ODN (943) | cggatgtgatcgatagt |
| OPN AS ODN (943) | actatcgatcacatccg |

### Histology

Tissue was fixed in 10% formalin or 4% paraformaldehyde for embedding in paraffin or Tissue-Tec® O.C.T. compound respectively. Sections (6 μm) were subjected to H&E, Masson's Trichrome, Toluidine Blue or Picrosirius Red staining, F4/80, or CD31 IHC (StreptABComplex/HRP, DAKO), MPO IHC (Envision, DAKO), or F4180-OPN double immunofluorescence. Antibodies used include the following:

| Primary Ab# | Species | Dilution | Blocking† | Secondary Ab* | Dilution |
|---|---|---|---|---|---|
| Osteopontin | Goat | 1:5000 (WB) | 5% milk | α-goat HRP (Jackson) | 1:5000 |
| (R&D Systems) | | 1:400 (IHC-p) | 10% Rb serum | Rabbit biotinylated α- | 1:600 |
| | | | | goat IgG (DAKO) | 1:400 |
| | | 1:400 (IHC-f) | 2% BSA | α-goat CY3 (Jackson) | |
| Myeloperoxidase | Rabbit | 1:200 (IHC-p) | 2% BSA | Envision system | |
| (NeoMarkers) | | | | (DAKO) | |
| F4/80 (Abcam) | Rat | 1:400 (IHC-p) | 10% Rb serum | Rabbit biotinylated α-rat | 1:300 |
| | | | | IgG (DAKO) | 1:400 |
| | | 1:400 (IHC-f) | 2% BSA | α-rat Alexa Fluor ® 488 | |
| | | | | (Molecular Probes) | |
| CD31 (Abcam) | Rat | 1:200 (IHC-p) | 10% Rb serum | Rabbit biotinylated α-rat | 1:300 |
| | | | | IgG (DAKO) | |
| Fibronectin | Rabbit | 1:400 (IHC-f) | 10% goat serum | Goat α-rabbit FITC | 1:1000 |
| (Abcam) | | | | (Abcam) | |
| α-smooth muscle | Rabbit | 1:5000 (WB) | 1% blocking | α-rabbit HRP (Sigma) | 1:3000 |
| actin (Abcam) | | | reagent (Roche) | | |
| Tubulin (Serotec) | Rat | 1:500 (IHC-f) | 2% BSA | α-rabbit CY3 (Jackson) | 1:400 |
| | | 1:2000 | 5% milk | α-rat HRP (Jackson) | |
| GAPDH | Rabbit | 1:5000 | 5% milk | n/a (primary Ab is HRP- | 1:5000 |
| (Abcam) | | | | conjugated) | |

Abbreviations:
Ab, antibody;
WB, Western Blotting,
IHC, Immunohistochemistry;
f frozen section;
p, paraffin section.
#All primary Abs were reacted overnight at 4° C.
†Blocking time is for 2 h at room temperature.
*All secondary Abs were reacted for 1 h at 37° C.

### Transmission Electron Microscopy (TEM)

Specimens were fixed at 4° C. overnight in 2% gluteraldehyde and 2% paraformaldehyde in 0.1 M sodium cacodylate buffer and subsequently for 1 h in 1% osmium tetroxide and then processed for TEM (Philips CM100). Details of collagen bundle density and fibre diameter measurements are outlined in supplementary text.

### RNA Isolation and RT-PCR Analysis.

Total RNA was extracted using TRIzol (Invitrogen) and further purified using RNeasy MinElute Cleanup Kit (Qiagen). RNA (5 μg) was reverse-transcribed (SuperScriptIII; Invitrogen), and the expression of OPN and GAPDH was analysed by standard PCR (HotStarTaq®; Qiagen; described in supp. text). TaqMan® Gene Expression Assays (Applied Biosystems) were used for amplification and real-time detection of transcript abundance.

### Western Blotting

Protein samples (20 μg; collected in T-PER® Reagent; PIERCE Biotechnology) were separated on Tris-Glycine Gels (Invitrogen), transferred to PVDF, and blotted according to standard protocols (antibody details are provided above). Protein bands were visualised by chemiluminescence (Roche Diagnostics GmbH) and band intensity was calculated using Image J 1.34s software.

### Cell Culture

Swiss 3T3, J774.1 macrophages, and RBL mast cells were cultured in DMEM+10% fetal calf serum. Primary neutrophils were harvested by peritoneal lavage 4 h post-thioglycollate-induced peritonitis, and primary macrophages were derived from mouse bone marrow and activated with LPS (500 ng/ml). Both primary cell types were maintained in RPMI 1640-1-10% serum. Conditioned media (CM) was prepared by culturing cells (2.5×10⁵ cells/ml) under standard conditions for 48 h, and media was then filtered (0.2 μm). Swiss 3T3 cells were treated with CM, or the following growth factors/cytokines for 8 h or periods thereof: OPN, PDGF-BB, EGF, TGFβ1 or 3, IL-G, TNF-α, Gleevec (STI571, imatinib mesylate; Novartis), wortmannin, or PDGF-Rβ neutralising antibodies as follows:

### Details of Growth Factors/Cytokines/Inhibitors Used.

| | | Concentration | |
|---|---|---|---|
| Product | Supplier | used | |
| PDGF-BB | R&D Systems | 20 | ng/ml |
| EGF | R&D Systems | 100 | ng/ml |
| TGFβ1 | BioVision | 10 | ng/ml |
| TGFβ3 | BioVision | 10 | ng/ml |
| IL-6 | BioSource | 50 | ng/ml |
| TNFα | BioSource | 10 | ng/ml |
| Gleevec | Novartis | 2-20 | μM |
| Wortmannin | Sigma | 100 | nM |
| PDGF-Rβ nAb | eBiosciences | 2 | μg/ml |

### Measurement of Epithelial Tongue Length, Epithelial Gap, and Width of Wound

Length of epithelial tongue, epithelial gap, and width of wound, as depicted in FIG. 2 D, were measured on H&E-stained tissue sections using Openlab™ 4.0.2 (Improvision) software.

### Transmission Electron Microscopy Analysis

For measurement of collagen diameter, at least five randomly selected fields of view per treatment group (magnification=28500×; depth below skin surface=360 μm) were photographed from intact and wound tissue, the fibril perimeters were measured using Image J software, and diameter was calculated (circumference/3.14). For measurement of collagen density, eight random fields of view (magnification=3900×) were photographed in intact and wounded skin, and the area of extra-cellular space not filled by collagen fibres (nm²/μm²) was calculated [number of white pixels in extracellular space converted to area (nm²)/(total field area (46 μm)−area occupied by cells), with the cellular area delineated using the free-hand drawing tool in Adobe PhotoShop software.

### Example 2

This example describes the design and optimization of candidates for AS ODNs.

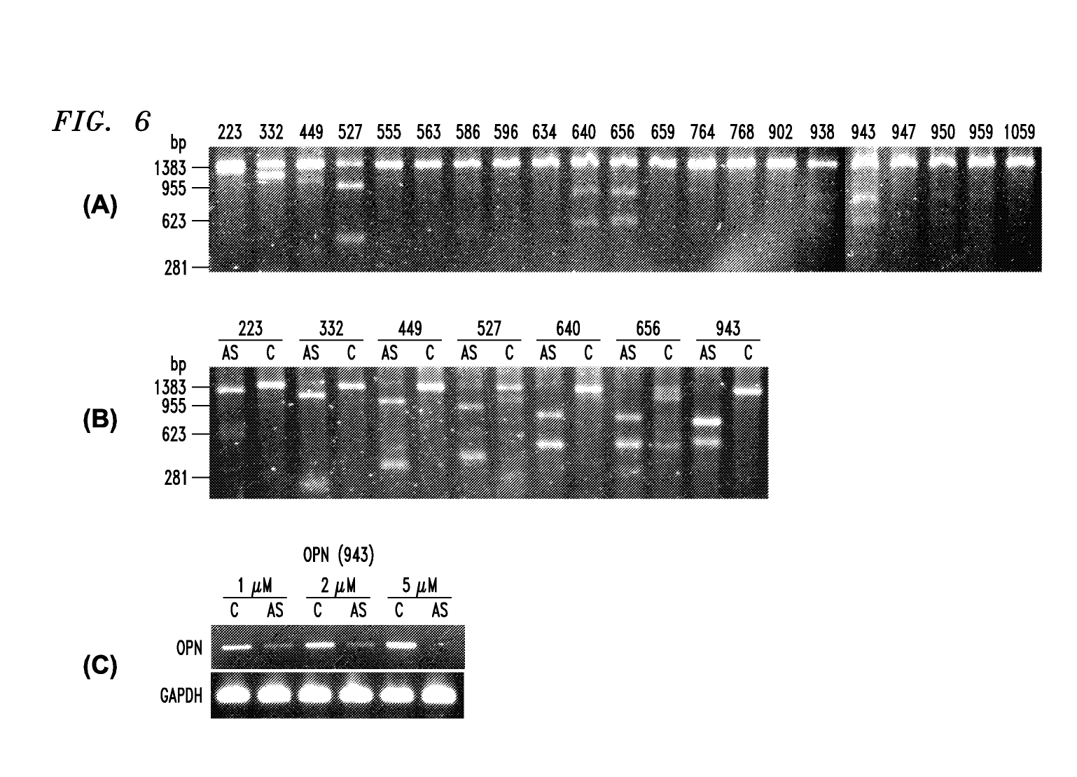

Design of candidate for AS ODNs for knockdown studies: Deoxyribozymes (Dzs) were designed as previously described (Martin, P., and S. J. Leibovich (2005) Trends Cell Biol 15:599-607). Briefly, the target sense OPN mRNA sequence (GenBank; NM 009263) was scanned for AT and GT sites, and Dzs were then generated to include eight nucleotides on either side of the AT or GT, and a catalytic core of “ggctagctacaacga”. BLAST searches were conducted to exclude any sequences non-specific to OPN. Antisense deoxyribonucleotides (AS ODNs) were subsequently designed based on the sequences for binding arms of successful Dzs (FIG. 6). The thermo-stabilities of the chosen AS ODNs (i.e., hair-pin and homodimer formation) were tested by OligoWalk (2). Sequence for Dzs-small letters represent the binding arm and capital letters indicate the catalytic core site (GGCTAGCTACAACGA):

| Name | Sequence |
|---|---|
| OPN Dz (223) | caatctca GGCTAGCTACAACGA ggtcgtag |
| OPN Dz (332) | ctatagga GGCTAGCTACAACGA ctgggtgc |
| OPN Dz (449) | cattggaa GGCTAGCTACAACGA tgcttgga |
| OPN Dz (527) | ccacagaa GGCTAGCTACAACGA cctcgctc |
| OPN Dz (555) | tggtgaga GGCTAGCTACAACGA tcgtcaga |
| OPN Dz (563) | catccgaa GGCTAGCTACAACGA ggtgagat |
| OPN Dz (586) | agcagtga GGCTAGCTACAACGA ggtctcat |
| OPN Dz (596) | cttgtgta GGCTAGCTACAACGA tagcagtg |
| OPN Dz (634) | gacatcga GGCTAGCTACAACGA tgtaggga |
| OPN Dz (640) | gttgggga GGCTAGCTACAACGA atcgactg |
| OPN Dz (656) | agctatca GGCTAGCTACAACGA ctcggccg |
| OPN Dz (659) | ccaagcta GGCTAGCTACAACGA cacctcgg |
| OPN Dz (764) | tagactca GGCTAGCTACAACGA cgctcttc |
| OPN Dz (768) | tccttaga GGCTAGCTACAACGA tcaccgct |
| OPN Dz (902) | ctttggaa GGCTAGCTACAACGA gctcaagt |
| OPN Dz (938) | cgatcaca GGCTAGCTACAACGA ccgactga |
| OPN Dz (943) | actatcga GGCTAGCTACAACGA cacatccg |
| OPN Dz (947) | cttgacta GGCTAGCTACAACGA cgatcaca |
| OPN Dz (950) | ttgcttga GGCTAGCTACAACGA tatcgatc |
| OPN Dz (959) | ctttggaa GGCTAGCTACAACGA ttgcttga |
| OPN Dz (1059) | attcggaa GGCTAGCTACAACGA ttcagata |

Sequences for Control, and AS ODNs:

| Name | Sequence |
|---|---|
| OPN control ODN (223) | ctacgaccatgagattg |
| OPN AS ODN (223) | caatctcatggtcgtag |
| OPN control ODN (332) | gcacccagatcctatag |
| OPN AS ODN (332) | ctataggatctgggtgc |
| OPN control ODN (449) | tccaagcaattccaatg |
| OPN AS ODN (449) | cattggaattgcttgga |
| OPN control ODN (527) | gagcgaggattctgtgg |
| OPN AS ODN (527) | ccacagaatcctcgctc |
| OPN control ODN (640) | cagtcgatgtccccaac |
| OPN AS ODN (640) | gttggggacatcgactg |
| OPN control ODN (656) | cggccgaggtgatagct |
| OPN AS ODN (656) | agctatcacctcggccg |
| OPN control ODN (943) | cggatgtgatcgatagt |
| OPN AS ODN (943) | actatcgatcacatccg |

Dz cleavage of OPN mRNA in vitro: Mouse OPN mRNA was transcribed from the IMAGE clone (GenBank, BC057858; (Geneservice Ltd), and resulting RNA was purified with RNeasy MinElute Cleanup Kit (Qiagen). RNA (0.3 μg/5 μl) was incubated in the cleavage buffer (10 mM MgCl2, 5 mM Tris (pH 7.5), 150 mM NaCl) for 5 min at 37° C., as was 5 μl of Dzs (40 μM; MWG Biotech). These were then combined and incubated for 1 h. RNA products were separated on 2% formaldehyde-agarose gel and stained with SYBR Gold nucleic acid gel stain (Invitrogen) to determine cleaving efficiency (FIG. 6 A).

AS ODN cleavage in vitro: To evaluate the cleaving efficiency of the AS ODNs, transcribed RNA (0.3 μg) was incubated with control or OPN AS ODNs (final concentration of 2 μM; MWG Biotech), RNase H (Invitrogen. Corp.), and RNasin (to inhibit non-specific RNA degradation; Promega) in a final volume 10 μl of cleavage buffer, and incubated for 20 min at 37° C. RNA products were separated on 2% formaldehyde-agarose gel and stained with SYBR Gold nucleic acid gel stain (Invitrogen Corp.) to determine cleaving efficiency (FIG. 6 B).

ODN delivery in vivo: ODNs were topically applied immediately post-wounding [50 μl; 1, 2, or 5 μM in 30% Pluronic F-127 gel, which is liquid at <4° C., but a firm gel at 37° C. that acts as a slow release vehicle (3); Sigma-Aldrich].

RT-PCR analysis for OPN transcript: Having determined that OPN AS ODN (943) was most effective at cleaving OPN mRNA, an in vivo experiment was conducted to determine the optimal dosing for knock-down. ODNs, control or OPN AS 943 (50 μl; 1, 2, or 5 μM in 30% Pluronic F-127 gel), were topically applied to full-thickness excisional wounds immediately post-wounding, and tissue was harvested for RNA 6 h later for RT-PCR analysis. Synthesized cDNA (Superscript III; Invitrogen) was amplified using HotStarTaq® DNA polymerase (Qiagen) and specific primers for target genes as follows: OPN (F) 5′-ACAAGACATCAACTGTGC-3′, OPN(R) 5TGACCTCAGAAGATGAACTCT-3′, GAPDH (F) 5′-ACTTTGTCAAGCTCATTTCC-3′, GAPDH (R) 5′-TGCAGCGAACTTTATTGATG-3′. Amplification was performed for 30 cycles with annealing at 55° C. (OPN) or 60° C. (GAPDH) for 30 s, extension at 72° C. for 1 min, and a final extension for 5 min. PCR products were separated by electrophoresis on a 2% agarose gel stained with ethidium bromide. Band sizes of OPN and GAPDH were 931 bp and 267 bp, respectively.

### Example 3

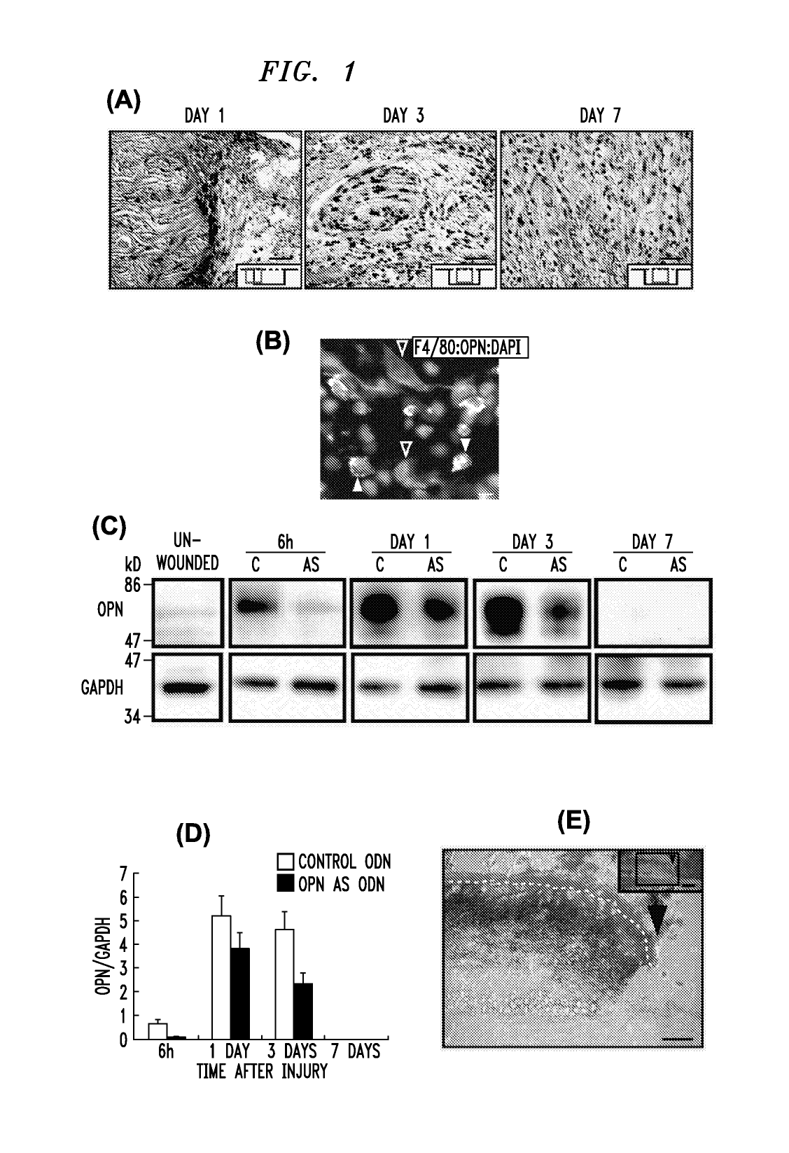

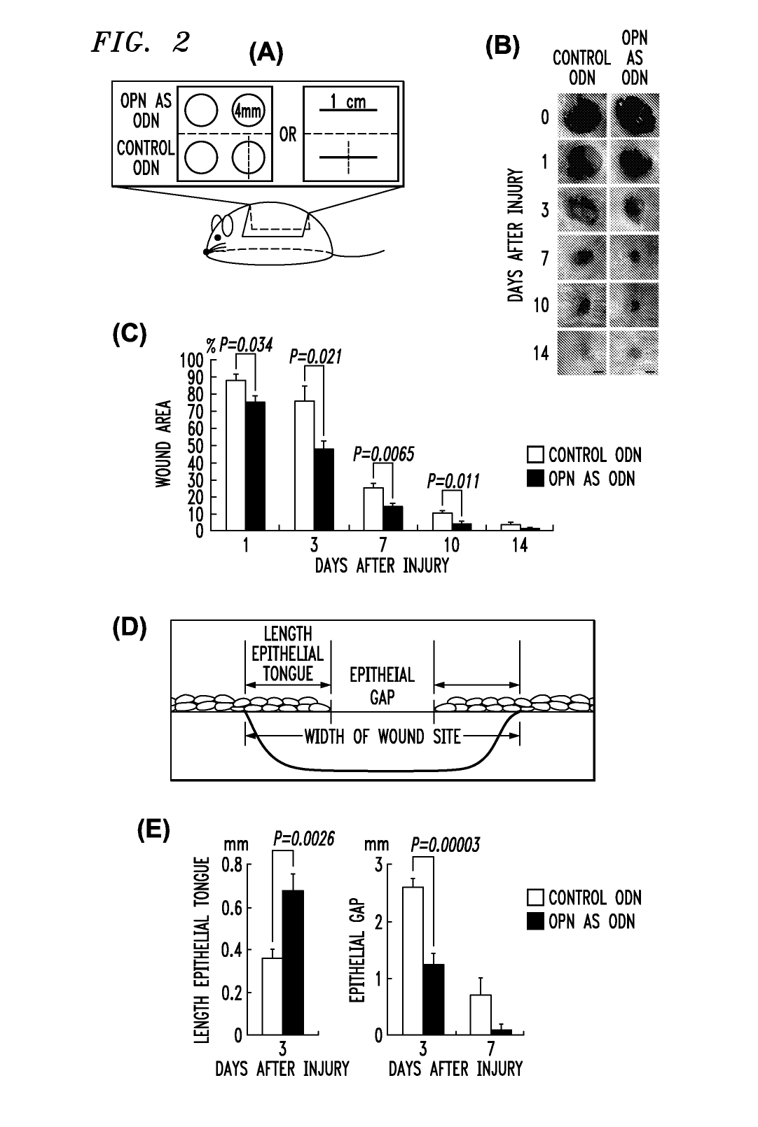

This example demonstrates the effects of down-regulating osteopontin (“OPN”) expression on wound healing, fibrosis, and scarring. Antisense oligodeoxynucleotides (“AS ODNs”) or a corresponding scrambled-sequence control ODN as set forth in the table above, were applied to in vivo 4 mm punch biopsy wounds on the backs of adult mice, at a concentration of 1 μM in 30% Pluronic gel. Typically, OPN is expressed in the wound granulation tissue by 6 hr post-wounding, with levels peaking at 3 days, and then diminishing to pre-wound levels by 7 days (FIG. 1A).

Immunofluorescence studies with antibodies against OPN and the macrophage-specific marker F4/80 indicate that whilst some wound macrophages express OPN at a low level, the majority of OPN positive cells are F4180 negative and thus mesenchymally derived and most likely are fibroblasts, although some may be pericytes (FIG. 1B). Western blot analyses of wound tissues indicate that the AS knock-down reproducibly reduced OPN levels to approximately 25% of control levels at 6 h, and 50% of control levels by 3 days post-wounding (FIGS. 1, C and D). This reduction was consistent with reduced immunohistochemical (IHC) staining for OPN in sections from AS ODN-treated wounds at these time-points. To visualize the uptake and localization of delivered ODNs, we generated a Cy3-tagged OPN AS ODN that shows uptake in a non cell-lineage specific manner, extending up to 1 mm back from the wound margin and in cell layers up to 300 μm deep within the wound (FIG. 1D).

### Example 4

This example demonstrates that OPN AS ODN improves rate and quality of wound healing

Macroscopic analyses of time-matched OPN knock-down versus control wounds showed that closure is markedly accelerated at early time-points during repair in OPN AS ODN-treated wounds (FIGS. 2, B and C), with a mean of 56% of complete closure achieved by 3 days compared to 24% closure in controls. Histological analysis revealed the relative contributions made by re-epithelialisation and connective tissue contraction (FIGS. 2 D) and the data indicates that re-epithalialisation, at least up to 3 days, is significantly faster in OPN AS ODN-treated wounds (FIG. 2 B). The data also indicate that that connective tissue wound width is less than controls at all stages during the healing process, suggesting enhanced connective tissue contraction from the earliest time-points studied (FIG. 2 F). In concord with this data, α-smooth muscle actin, a marker of contractile myofibroblasts, is expressed on day 3 in OPN AS ODN-treated wounds sites, which is earlier than in control wounds (FIG. 2 G). The fact that AS ODN-treated wounds are capable of complete closure demonstrates that OPN is not absolutely required in skin repair, which is consistent with observations of wound repair in OPN-null mice (Liaw, L., D. E. Birk, C. B. Ballas, J. S. Whitsitt, J. M. Davidson, and B. L. Hogan. 1998. Altered wound healing in mice lacking a functional osteopontin gene (sppl). J Clin Invest 101:1468-1478). This data also counters the idea that lower OPN expression observed in a diabetic mouse model is functionally responsible for slower wound healing (Sharma, A., et al. (2006) J Invest Dermatol 126:2323-2331). Thus, OPN expressed by wound fibroblasts as a consequence of signals from inflammatory cells can hinder the rate of repair.

Sections of excisional wounds were stained with Masson's Trichrome in order to reveal the cross-sectional area of wound granulation tissue in the mid-wound region (FIG. 2 G). At all time-points analysed OPN knock-down wounds had considerably reduced cross-sectional area of granulation tissue compared to controls (at 10 and 14 days, 50% less; FIGS. 2 H and I). These data implicate OPN expression at the wound site as being, at least partially, causal of the extensive granulation tissue formation and subsequent fibrosis that we see during repair of standard skin lesions the data do not distinguish whether OPN might be directly responsible for these changes or whether they are an indirect consequence of the reduced inflammatory response. Although expression of OPN has previously been correlated with several fibrotic conditions in other anatomical sites [e.g. lung (Pardo, A., K. et al. (2005) PLoS Med 2:e251.), liver (Lee, S. H. et al. (2004) Biochem Pharmacal 68:2367-2378.), heart (Matsui, Y., et al. (2004) Hypertension 43:1195-1201.)], this study of skin repair is the first functional demonstration that OPN expression may contribute to fibrosis.

Measurement of epithelial tongue length, epithelial gap, and width of wound

Length of epithelial tongue, epithelial gap, and width of wound, as depicted in FIG. 2 D, were measured on H&E-stained tissue sections using Openlab™ 4.0.2 (Improvision) software.

### Example 5

This example demonstrates altered collagen organization at OPN AS ODN-treated wound sites.

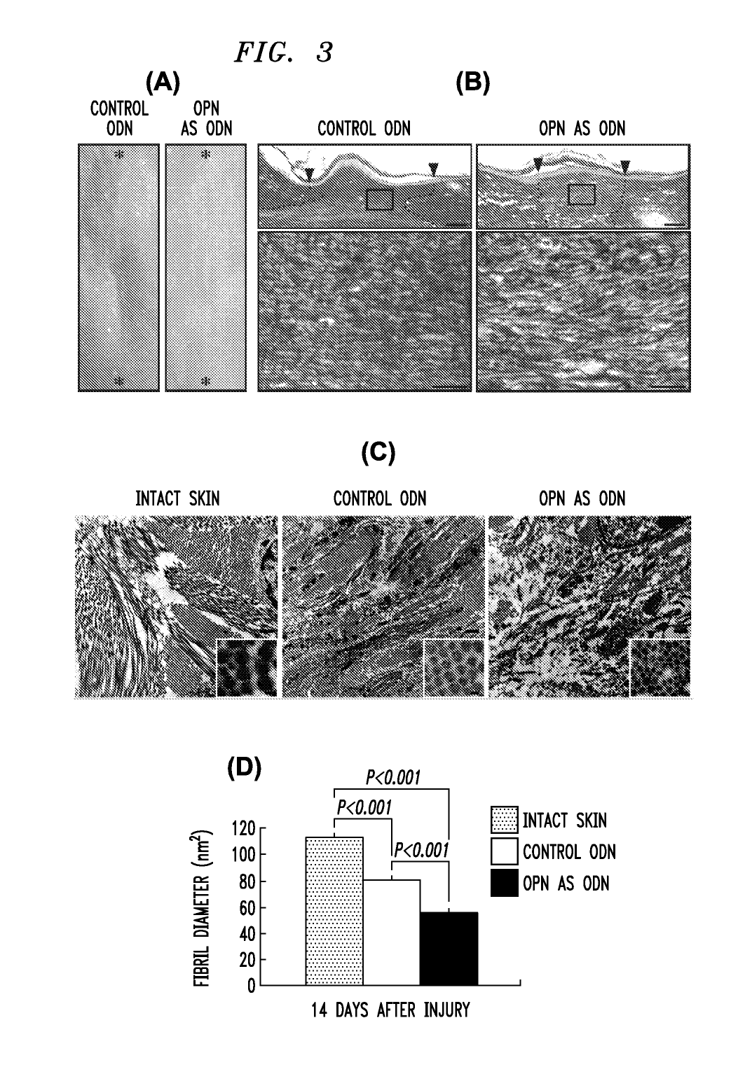

To address whether knock-down of OPN influences the level of scarring resulting from the repair process, we used a 1 cm incisional wound model, which were monitored daily for up to 3 weeks. Macroscopic analyses 21 days after injury showed that the control wounds developed a thin linear white scar, which was noticeably reduced in OPN knock-down wounds (FIG. 3 A). To further compare developing scar tissue in control- and OPN AS ODN-treated wounds, Picrosirius Red staining and TEM analysis of 3 week wound sections were undertaken to reveal gross collagen bundling patterns and ultra-structural analysis of individual collagen fibril diameter and density of fibrils in wound connective tissues, respectively (FIG. 3, B, C, D). The Picrosirius Red histology revealed less dense “scar collagen” in the OPN AS ODN-treated wounds (FIG. 3 B), and the ultrastructural studies showed more “vacant” extra-cellular space (i.e., space between the collagen fibrils and between bundles), such that 153 nm²/μm² of the cross-sectional area through the mid-region of AS ODN-treated wounds was “empty” of collagen fibers by comparison to 114 nm²/μm² in control ODN-treated wounds, and 132 nm²/μm² in unwounded skin (FIG. 3 C). Moreover, a significant reduction in collagen fibril diameter was observed in OPN AS ODN-treated wounds in comparison to controls, both of which displayed smaller diameters than unwounded skin (56 nm vs. 81 nm vs. 114 nm, respectively; FIGS. 3, C and D). These differences in collagen fibril diameter and bundling density at the wound site may reflect and give rise to the later differences in extent of fibrosis seen in OPN AS ODN-treated wounds.

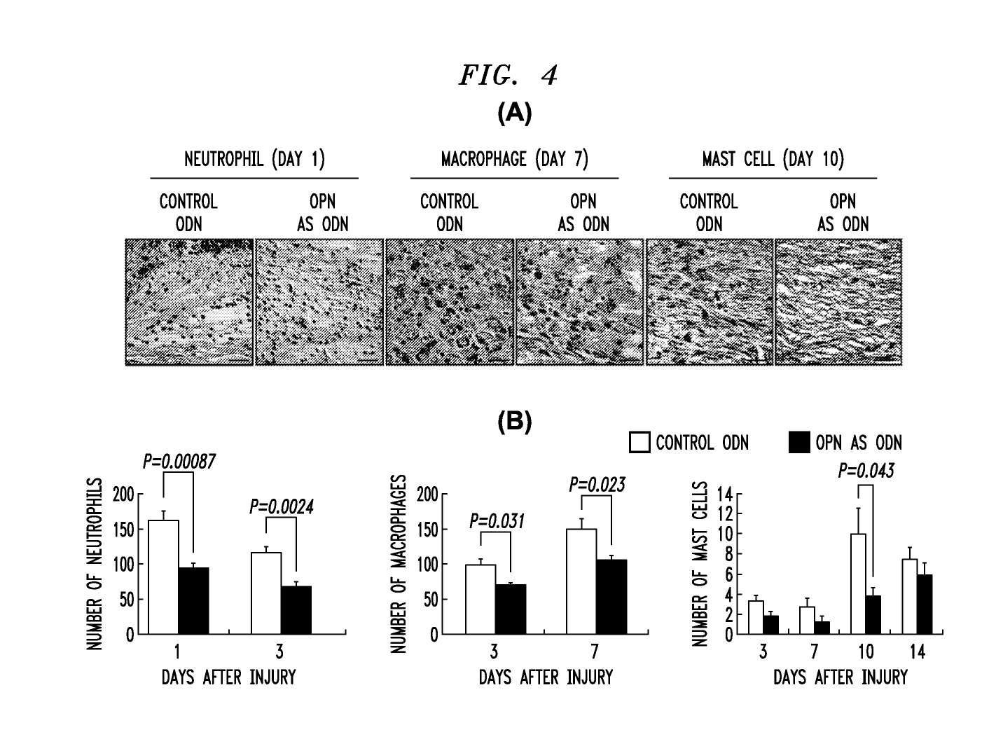

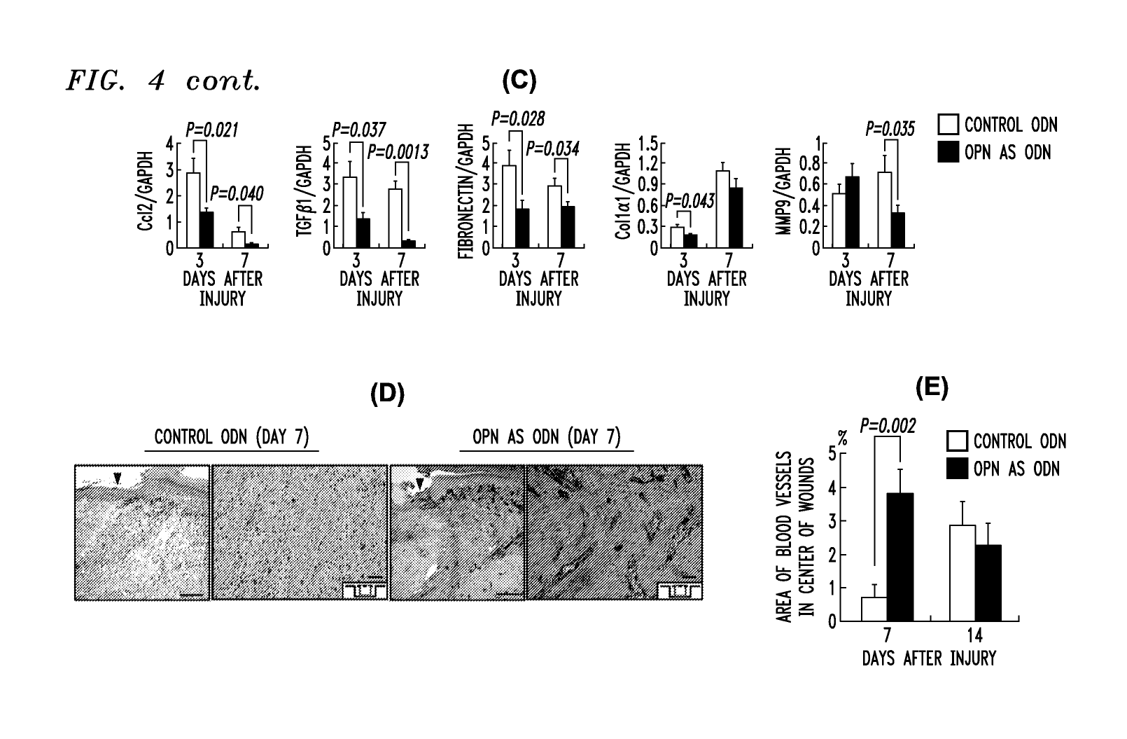

Collagen fibrillogenesis is known to be regulated by several enzymes, including members of the ADAMTS family, BMP1, and the mammalian tolloids, as well as by interaction with extracellular matrix proteins (Canty, E. G., and K. E. Kadler. (2005) J Cell Sci 118:1341-1353.). The data described herein, together with data from OPN-null mice where the diameter of collagen fibrils at deep dermal wound sites are also smaller than in wild-type mice (Liaw, L., et al. (1998) J Clin Invest 101:1468-1478), demonstrates that this process may also be affected by OPN. It has been reported that OPN interacts directly with collagen types I and III, which are the chief isoforms of intact and repairing skin connective tissue, respectively (Butler, W. T. (1995) Ann N Y Acad Sci 760:6-11.). Alternatively, OPN could affect collagen fibril formation indirectly, for example by altering fibronectin levels, which we demonstrate are reduced in AS OPN-treated wounds (FIG. 4 C). Studies of fibronectin-null fibroblasts have shown that the interaction of these two molecules is a key step in collagen assembly (Veiling, T., J. Risteli, K. Wennerberg, D. F. Mosher, and S. Johansson. (2002) J Biol Chem 277:37377-37381).

### Example 6

This example demonstrates that leukocyte recruitment is significantly reduced in OPN AS ODN-treated wounds

Several lineages of inflammatory cells are recruited to sites of tissue damage with overlapping time-courses during the repair process. Both neutrophils and macrophages are known to influence various aspects of repair and we observe that the numbers of both these cell types are significantly reduced as a consequence of OPN AS ODN treatment at times when their numbers are peaking in control wounds (i.e., 1 day for neutrophils and 7 days for macrophages; FIGS. 4, A and B). This data is consistent with other in vivo evidence that OPN can function as a chemoattractant for neutrophil and macrophage lineages (Giachelli, C. M., et al. (1998) Am J Pathol 152:353-358). Mast cells, which are also implicated in tissue remodeling at wound sites (Egozi, E. I. et al. (2003) Wound Repair Regen 11:46-54), exhibit reduced numbers by comparison to control wounds at 10 days (FIGS. 4, A and B). Associated with these reduced numbers of inflammatory cells at the wound site are reduced levels of mRNA encoding the potent inflammatory cell chemoattractant, Ccl2, as well as TGFβ1 (FIG. 4 C), indicates that a positive feedback loop leading to amplification of the inflammatory episode may be disrupted by OPN knock-down.

### Example 7

This example demonstrates altered extracellular matrix deposition and angiogenesis in OPN AS ODN-treated wounds

The reduced extent of granulation tissue in OPN knock-down wounds and the associated down-regulation of TGFβ1 [a fibrogenic factor during skin wound healing (Frank, S., M. Madlener, and S. Werner. (1996) J Biol Chem 271:10188-10193.)] led us to the investigation of the alterations in matrix deposition at the wound site. We show that fibronectin and type I collagen al mRNA are reduced at OPN AS ODN-treated wounds, and that the wound induced metalloproteinase, MMP9, is also expressed at significantly lower levels from 7 days on (FIG. 4 C).

Angiogenesis is intimately associated with granulation tissue formation during wound repair. We have evaluated angiogenesis by IHC for Platelet/endothelial cell adhesion molecule 1 (PECAM; CD31) in control and OPN AS ODN wounds. OPN AS ODN led to significantly increased numbers of vessel lumens in the mid-granulation tissue zone at 7 days post-wounding compared to control wounds (FIGS. 4 D and E). This difference in the extent of wound vascularity was resolved by 14 days, and indicates an increased rate of vessel invasion at early time-points in the repair process. This appears not to be due to altered levels of the wound angiogenic factor VEGF since we see no significant difference in OPN AS ODN-treated wounds. Recent evidence suggests that OPN has the capacity to directly inhibit angiogenesis (Leali, D., E. Moroni, F. Bussolino, and M. Presta. (2007) J Biol Chem), but we also note that during repair of skin wounds in PU.1-null mice, where there is no inflammatory response and reduced scarring, there is also significantly increased vascularity within wound granulation tissue (Martin, P., et al. (2003) Curr Biol 13:1122-1128). Similarly, TNF-Rp55 knock-out mice that lack TNF-α-mediated signaling, display increased angiogenesis at skin wound sites (Mori, R., et al. (2002) FASEB J 16:963-974). These results indicate that the inflammatory response and inflammatory cytokines may negatively regulate angiogenesis, and this may encourage fibrosis during normal wound repair.

### Example 8

This example demonstrates the inflammation-dependent regulation of OPN expression is mediated largely by macrophages

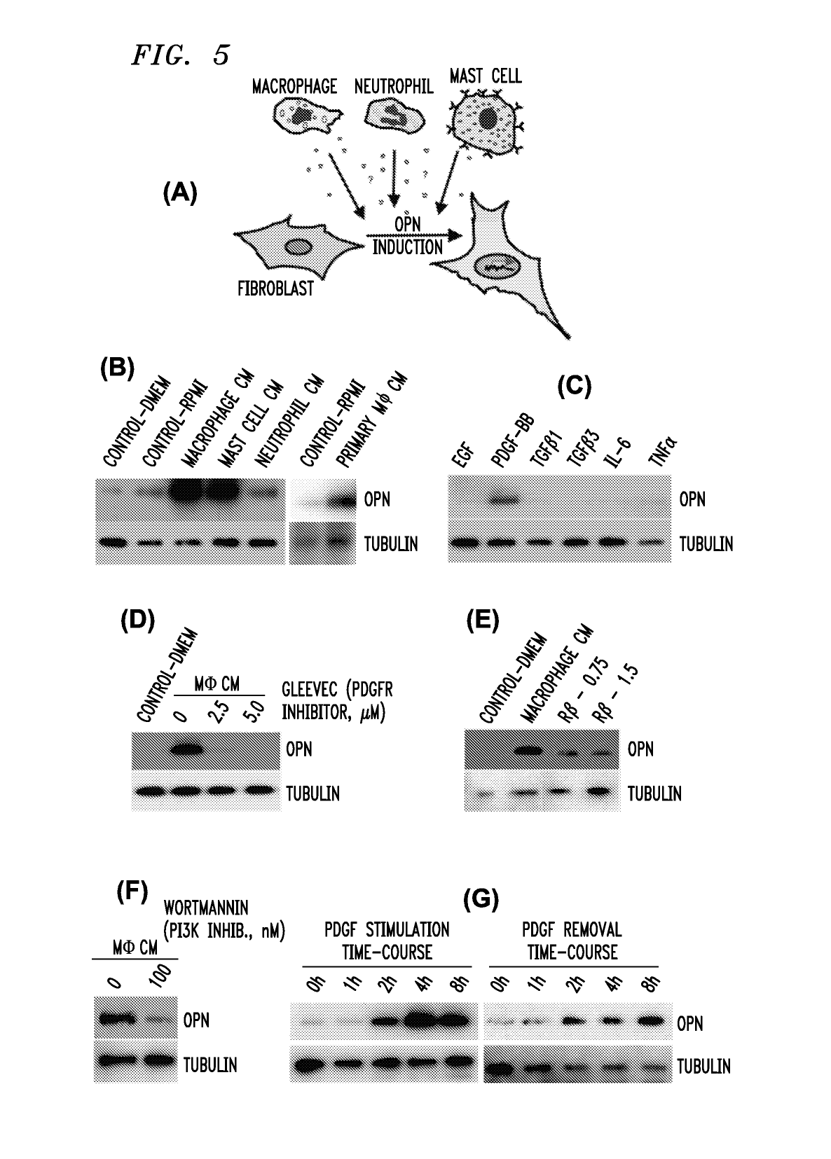

The comparison of the gene expression profiles of tissue repair in wild-type versus PU.1-null mice revealed that OPN is an inflammation-dependent repair gene, but since the PU.1-null mice lack neutrophils, macrophages, and mast cells, it was unclear which of these leukocytic lineages might be responsible for inducing OPN expression in the normal repair situation. To address this, Swiss 3T3 fibroblasts were cultured in the presence of conditioned media (CM) from equal numbers of J774.1 macrophages, primary mouse neutrophils, RBL mast cells, or primary bone marrow-derived macrophages (FIG. 5 A). Western blot analysis showed that factors secreted by all macrophages tested, and mast cells, but not neutrophils, can trigger 3T3 fibroblast expression of OPN (FIG. 5 B). Normal human dermal fibroblasts similarly respond to macrophage-conditioned media with a robust up-regulation of OPN. Induction of OPN expression in in vivo wounds coincides temporally with the first influx of macrophages to the wound, whereas mast cell numbers peak much later following injury, and are relatively few in number throughout repair [(Egozi, Ed., et al. (2003) Wound Repair Regen 11:46-54), Trautmann, A., et al. (2000) J Pathol 190:100-106.) and FIG. 4]. Since macrophages are likely to be providing the major inductive signal for OPN up-regulation during in vivo healing, these data do not rule out a contribution from mast cells. Six growth-factors/cytokines known to be secreted by macrophages (Rappolee, D. A. et al. (1988) Science 241:708-712.) were then tested for their ability to induce OPN expression in Swiss 3T3s. PDGF-BB, but not EGF, TGFβ1, TGFβ3, IL-6, or TNF-α, was able to significantly upregulate OPN expression (FIG. 5 C). To confirm that PDGF was indeed mediating the macrophage-CM induction of OPN, we suppressed PDGF-R signaling with Gleevec or PDGF-Rβ neutralizing antibodies during fibroblast exposure to macrophage CM (FIGS. 5, D and E), and showed that OPN expression was significantly reduced. Induction of OPN could similarly be blocked with wortmannin (FIG. 5 F), which implicates the PI3K signaling cascade down-stream of PDGF in transducing this signal. In an in vivo setting, release of PDGF by degranulating platelets occurs immediately but transiently following injury, and subsequently is supplemented by local up-regulation by resident wound cells and invading macrophages.

We performed a time-course experiment to address the minimum window of exposure to PDGF required for OPN expression and the data suggest that OPN levels peak about 8 h after initial exposure and require at least a 2 h window of exposure (FIG. 5 G). These results, taken together with the observation that PU.1-null mice, which have normal platelet biology but do not express OPN in wounds (Cooper, L., C. Johnson, F. Burslem, and P. Martin. 2005. Genome Biol 6:R5.), suggest that platelet-derived PDGF exposure is not sufficient on its own to induce OPN expression; rather, sustained exposure of fibroblasts to macrophage-derived PDGF is responsible for the OPN induction.

While OPN expression at sites of tissue repair appears to have largely detrimental consequences on healing, the same is not true for PDGF, as highlighted by the observation that Gleevec treatment delivered from the outset of the repair process significantly slows wound closure (Rajkumar, V. S., et al. (2006) Am J Pathol 169:2254-2265.). Indeed, topical application of PDGF-BB to wounds has been shown to accelerate the healing of chronic ulcers (Chan, R. K., et al. (2006) J Burn Care Res 27:202-205.), but there is evidence that this improvement in healing speed is accompanied by increased granulation tissue and scarring (Lynch, S. E., J. C. Nixon, R. B. Colvin, and H. N. Antoniades. (1987) Proc Natl Acad Sci USA 84:7696-7700.). These negative side-effects may be attributable to PDGF-induced OPN expression, and that down-regulation of OPN may be a molecular mechanism by which Gleevec reduces pulmonary and dermal fibrosis (Distler, J. H., et al. (2007) Arthritis Rheum 56:311-322, Aono, Y., et al. (2005) Am J Respir Grit Care Med 171:1279-1285.). Studies indicate that neither TGFβ1 nor β3 is the inductive signal responsible for triggering OPN expression by wound fibroblasts; yet acute knock-down of OPN improves both the rate of healing and the eventual quality of the repair.

Together, these in vivo wound studies and tissue culture experiments indicate that inflammatory cell-mediated signals such as, for example, macrophage-derived PDGF, can trigger the expression of OPN in wound fibroblasts, and this can both retard repair and cause the fibrosis resulting from wound healing. The OPN expression can lead to increased fibrosis via pleiotropic effects, influencing wound angiogenesis, amplifying the inflammatory response, and even directly modulating the expression of matrix genes in fibroblasts.

### Example 9

Methods of sequentially administering an anti-osteopontin AS ODN composition alone or in combination with an anti-connexin 43 polynucleotide preparation prepared with the following exemplary sequences: GTA ATT GCG GCA GGA GGA ATT GTT TCT GTC (connexin 43) (SEQ.ID.NO:2) and GAC AGA AAC AAT TCC TCC TGC CGC ATT TAC (sense control) followed by administration of an anti-osteopontin polynucleotide preparation prepared with the sequences from Table 10 are evaluated for the efficacy in wound healing in rat diabetic model.

Diabetes is induced in adult Sprague-Dawley rats (350-400 g) by a single intraperitoneal injection containing streptozotocin, 65 mg/kg, in citrate buffer (Shotton H R, Clarke S, Lincoln J. (2003)). The effectiveness of treatments of diabetic autonomic neuropathy is not the same in autonomic nerves supplying different organs (Id.). The effectiveness of treatments of diabetic autonomic neuropathy is not the same in autonomic nerves supplying different organs. (Id.). The effectiveness of treatments of diabetic autonomic neuropathy is not the same in autonomic nerves supplying different organs (Id.) (N=six diabetic, six control per time point). Most diabetic wound-healing studies are carried out two weeks after diabetes induction and the same time point is used for this wound healing study. However, connexin expression in diabetic rat skin is also examined at eight weeks (N=six diabetic, six control per time point) to confirm that the changes detected at two weeks will remain the same. Normal back skin is excised, cryosectioned, immunostained for connexins, imaged by confocal microscopy and the staining quantified as described in Saitongdee et al. (2000) Effects of hibernation on expression of multiple gap junction connexins in hamster myocardium, Cardiovascular Res. 47, 108-115.

Rats are anaesthetised with halothane and their backs are shaved. Two pairs of 5×5 mm full thickness excision wounds are made. 10 μM of the connexin 43 oligodeoxynucleotide GTA ATT GCG GCA GGA GGA ATT GTT TCT GTC (SEQ.ID.NO: 2) in Pluronic F-127 gel is applied one wound and control (sense) gel to the other.

1-μM of anti-ostepontin agents from Table 10 in Pluronic F-127 gel is applied to one wound and control (Pluronic F-127 gel only) applied to the second wound within 1 minute, 5 minutes, 10 minutes, 30 minutes, 1 hour or 6 hours.

Tissue is harvested on days 1, 2, 5, 10 and 15 after wounding, and sectioned in preparation for connexin immunohistochemistry or H&E staining (Coutinho P, et al. (2003) Dynamic changes in connexin expression correlate with key events in the wound healing process. Cell Biol. Int. 27:525-541). N=six diabetic, six control rats per time point.

Intercellular communication is assessed by applying a 4% solution of Lucifer Yellow CH (Sigma) in a pledget of gelfoam into a fresh, full thickness skin incision. Dye is allowed to transfer for 5 minutes prior to removal of the gel foam and fixation of the tissue. A 10 kD Kd FITC-dextran that will enter injured cells but not pass through gap junctions is used as a control. Tissues are cryosectioned and imaged by confocal microscopy on a Leica SP2UV (Leica, Milton Keynes, UK).

Transferred dyes and connexin immunostaining are examined using the confocal microscope. Optimal gain and offset are set in advance and kept constant during the image acquisition process. A series of single optical section images are taken to generate a montage of the skin from the cut. Digital images (eight bit) are analysed using Image-J software (NIH). To assess dye transfer, a 1500×30 pixel region-of-interest box is placed from the cut edge in the mid-dermis and an image intensity graph across the box is generated. A grey level intensity drop below 50 is taken as the point where Lucifer Yellow had traveled. Similarly, in the epidermis, the distance from the cut to where the Lucifer Yellow signal dropped below 50 is recorded. A minimum of three images are analyzed from each animal. To compare levels of connexin protein, six single optical section images of dermis or epidermis are taken from different sections for each wound. All parameters of laser power, pinhole, gain/offset and objective are kept constant across both control and diabetic groups. Connexin expression is quantified as described in Saitongdee P, et al. (2000), supra. A threshold is set to detect gap-junction plaques with minimal background noise and is then kept constant for all images. The number and size of connexin plaques are recorded for each image and expressed per 100 m of epidermis or 10000 m² of dermis. This approach has proved to be much more accurate than Western blot as it generates information on protein expression at the cellular level. Western blots are unable to distinguish between epidermal and dermal cells or detect effects of proximity to the wound edge. Using this approach, connexin levels in keratinocytes in a zone at the wound edge (WE) and in a zone 500 μm away (AD) are able to be quantified either one or two days after wounding. At day five after wounding, an additional zone of the leading edge (LE) of the nascent epidermis is also imaged. Images of H&E staining are taken using a Leica DMLFS microscope with a DC300F digital camera. Measurements for re-epithelialization rate are described in detail in Qiu, C et al. (2003) Targeting connexin43 expression accelerates the rate of wound repair. Curr. Biol. 13:1697-1703. All numerical differences between treatments are tested for significance using the Wilcoxon matched-pairs signed-ranks test as implemented in Statview 5.0.1.

Relative connexin 43 and 26 staining levels in normal and STZ diabetic rat skin at two weeks and eight weeks after induction of diabetes are measured and compared. Graphs are plotted to show the numbers of plaques in the epidermis and dermis. Images of typical connexin 43 and connexin 26 immunostaining in control and diabetic skin at eight weeks are acquired (arrowheads mark the boundary between the epidermis and dermis; scale bar 25 m). The relative distances that the gap-junction-permeant dye, Lucifer Yellow, traveled in five minutes in the epidermis and dermis of the control and diabetic rats are quantified.

Typically punctate connexin 43 immunostaining is found in the basal layer of the epidermis, and in dermal fibroblasts, hair follicles, blood vessels and appendages. However, in diabetic skin, connexin 43 staining may be significantly reduced in the epidermis, in terms of both size and number of gap junction plaques. Staining for connexin 26 in the upper layers of the epidermis may be similarly reduced in diabetic epidermis.

To assess cell-cell communication in diabetic epidermis and dermis, the extent of transfer of the gap-junction-permeant dye Lucifer Yellow through the tissue in five minutes is examined. Elevated expression of connexin 43 protein and increased communication has been reported in human diabetic fibroblasts (Abdullah K M, et al. (1999) Cell-to-cell communication and expression of gap junctional proteins in human diabetic and nondiabetic skin fibroblasts: effects of basic fibroblast growth factor, Endocrine 10:35-41); and mixed responses of different connexins to diabetes have been noted in the renal system (Zhang J, Hill C E. (2005) Differential connexin expression in preglomerular and postglomerular vasculature: accentuation during diabetes, Kidney Int. 68:1171-1185).

Relative rates of re-epithelialization and responses of connexin 43 and connexin 26 protein levels following injury in control and diabetic epidermis are measured. Staining is quantified by counting plaques at one and two days after wounding in epidermis at the wound edge (WE) and adjacent (AD) epidermis, 500 m away. On day five, an additional zone at the leading edge (LE) of the nascent epidermis is quantified.

Connexin 43 and connexin 26 staining (green) and nuclear staining (blue) at the epidermal wound edge of control and diabetic skin during the wound-healing process are measured and the processed by image analysis.

To determine the dynamic responses of connexin expression to injury, connexin staining in keratinocytes at the wound edge (WE) and in an adjacent zone 500 μm away (AD) is quantified at one and two days after wounding. At after five days the leading edge (LE) keratinocytes is imaged.

The effect of the possible increase of connexin 43 protein in diabetic WE keratinocytes is assessed by preventing the increase with a connexin 43-specific antisense gel, applied to the wound at the time of injury.

A finding of abnormal upregulation of connexin 43 in the epidermal wound edge in diabetes is significant, and has the potential to affect the process of wound closure in different ways. The formation of communication compartments within the regenerating epidermis has been proposed to play a role in wound healing (Martin P (1997) Wound healing—aiming for perfect skin regeneration, Science 276:75-81; Lampe P D, et al. (1998) Cellular interaction of integrin alpha3beta1 with laminin 5 promotes gap junctional communication. J. Cell Biol. 143:1735-1747; Hodgins M (2004) Connecting wounds with connexins. J. Invest. Dermatol. 122: commentary). Compartmentalization could be effectively brought about in normal conditions by expression of connexin 26 and removal of connexin 43 in leading edge cells, as these connexins do not form junctions with one another. Thus, the delay in wound healing in diabetes could reflect the additional time required for connexin 43 expression to downregulate to a point where such a compartmentalization can occur. Alternatively, the C-tail of connexin 43 is known to interact with cytoskeletal components or with P120ctn/Rho GTPase, so downregulation of connexin 43 could be necessary for changing the motility of keratinocytes at the wound edge, enabling them to migrate and close the wound (Wei C J, et al. (2004) Connexins and cell signaling in development and disease, Annu Rev Cell Dev Biol. 20:811-838).

### Example 10

Wound healing efficacy in a diabetic subject is investigated after sequentially administering an anti-osteopontin AS ODN composition alone or in combination with an anti-connexin 43 peptide preparation prepared with the following exemplary sequence: SRPTEKTIFII (SEQ.ID.NO.: 19) followed by administration of anti-connexin 43 polynucleotides preparation prepared with the following exemplary sequences: GTA ATT GCG OCA GGA GGA ATT OTT TCT GTC (connexin 43) (SEQ.ID.NO:2) and GAC AGA AAC AAT TCC TCC TGC CGC ATT TAC (sense control) (SEQ.ID.NO.:19) in vivo to diabetic male Sprague Dawley rats. In order to quantify the wound healing in a diabetic subject, the tensile strength of the wounds is investigated, with a higher tensile strength reflecting an improvement in wound healing.

The diabetic rat animal model is an established model system for investigating diabetes-associated wounds, which heal poorly (Davidson, Arch. Dermatol. Res. 290: S1-S11). Since diabetes is accompanied by microangiopathy, this animal model is also suitable for investigating arterially determined disturbances in wound healing.

In order to induce the diabetes, rats having a bodyweight of 250-300 g are injected i.p. with a freshly prepared aqueous solution of streptozotocin (Sigma) (50 mg/kg of bodyweight). The blood sugar of the animals is checked 7-9 days after induction, with a blood sugar level value of more than 200 mg/dL confirming the diabetic state. The diabetic rats and the nondiabetic control animals are subsequently anaesthetized with a mixture consisting of 2% O₂ (2 l/min) and 1.25% isofluran. The back is depilated and 2 sites are marked on the back of each animal for subsequent wounding. Incision wounds of 1 cm in length are then made through the wound sites and the wounds are closed with wound clips.

First, 1-5 μM of an anti-osteopontin AS ODN set forth in Table 10 in combination with 100-500 micrograms of anti-connexin 43 peptide SRPTEKTIFII (SEQ ID. NO: 19) in Pluronic F-127 gel is applied to one wound. A control (Pluronic F-127 gel only) is applied to the second wound. Thereafter, 10 μM of the connexin 43 oligodeoxynucleotide GTA ATT GCG GCA GGA GGA ATT GTT TCT GTC (SEQ.ID.NO:2) in Pluronic F-127 gel is applied to one wound and control (sense) gel to the other at either within 1 minute, 5 minutes, 10 minutes, 30 minutes, 1 hour or 6 hours.

The wound biopsies are taken after 10 days and the tensile strength of the wounds is determined using an Instron tensiometer in accordance with the manufacturer's instructions and standardized to the cross sectional area of the wounds.

Subsequently, the quotient (E/C value) is calculated from the absolute value of the tensile strength of a wound, which is treated and the absolute value of the tensile strength of a wound in the same animal which only receive the control preparation. The mean of the E/C values is determined and the changes in tensile strength relative to the treatment are determined.

### Example 11

Certain formulations of the invention are prepared made by combining an anti-osteopontin agent or a PDGF receptor inhibitor or both (optionally, together with an anti-connexin agent). These active ingredients are provided in therapeutically desired amounts, e.g., at a concentration of about 0.01%-10%, together with one or more of sodium carboxymethylcellulose; sodium chloride; sodium acetate trihydrate; glacial acetic acid; a preservative or a combination of preservatives, such as one or more or all of methylparaben, propylparaben, and m-Cresol; L-lysine hydrochloride (a protein stabilizer, where needed or desired); and, water for injection. The sodium content and pH are chosen to resemble physiological salt concentrations and to establish maximum stability of the active agent(s) respectively. The gel may be filled into laminated aluminum tubes with a polyethylene liner, to provide single- or multiple-use products. A preservative or a combination of preservatives, such as one or more or all of methylparaben, propylparaben, and m-Cresol, is used where a multiple-use product is prepared.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof; but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. An antisense polynucleotide to osteopontin protein for use in the treatment of the human or animal body by therapy.

2. A polynucleotide according to claim 1 which is an oligodeoxynucleotide.

3. A polynucleotide according to claim 1 or 2 which is single-stranded.

4. A polynucleotide according to claim 4 in which the polynucleotide is selected from CAA TCT CAT GGT CGT AG (SEQ.ID.NO:158); CTA TAG GAT CTG GGT GC (SEQ.ID.NO:159), CAT TGG AAT TGC TTG GA (SEQ.ID.NO:160), CCA CAG AAT CCT CGC TC (SEQ.ID.NO:161), GTT GGG GAC ATC GAC TG (SEQ.ID.NO:162), AGC TAT CAC CTC GGC CG (SEQ.ID.NO:163), or ACT ATC GAT CAC ATC CG (SEQ.ID.NO:164).

5. The use of at least one antisense polynucleotide to an osteopontin protein as defined in any one of claims 1 to 4 in the manufacture of a medicament for use in wound-healing.

6. The use of at least one antisense polynucleotide to an osteopontin protein as defined in any one of claims 1 to 4 in the manufacture of a medicament for reducing inflammation.

7. The use of at least one antisense polynucleotide to an osteopontin protein as defined in any one of claims 1 to 4 in the manufacture of a medicament for decreasing scar formation.

8. The use as defined in any one of claims 5 to 7 wherein the wound is an acute wound.

9. The use as defined in any one of claims 5 to 7 wherein the wound is a chronic wound.

10. A formulation for use in therapeutic or cosmetic treatment, which formulation comprises at least one antisense polynucleotide as defined in any one of claims 1-4 together with a pharmaceutically acceptable carrier or vehicle.

11. A formulation according to claim 10, suitable for topical administration.

12. A formulation according to claim 10 in which one of the osteopontin proteins to which polynucleotides are directed is a human osteopontin.

13. A formulation according to claim 10 in which one of the osteopontin proteins to which polynucleotides are directed is a non-human osteopontin.

14. A formulation according to claim 13 wherein the polynucleotide reduces expression of horse, dog or cat osteopontin.

15. A formulation according to any one of claims 10-14 in which the pharmaceutically acceptable carrier or vehicle is, or includes, a gel.

16. A formulation according to claim 15 in which the gel is a nonionic polyoxyethylene-polyoxypropylene copolymer gel.

17. A formulation according to any one of claims 10 to 16 which further includes a surfactant or urea to assist with polynucleotide penetration into cells.

18. A formulation according to any one of claims 10 to 16, which formulation is in the form of a cream, ointment, gel, emulsion, lotion, foam or paint.

19. A formulation according to any one of claims 10 to 16 which is a sustained release formulation.

20. A formulation according to any one of claims 10 to 16, which further comprises an auxiliary agent selected from casein, gelatin, albumin, glue, sodium alginate, carboxymethylcellulose, methylcellulose, hydroxyethylcellulose or polyvinyl alcohol.

21. A formulation according to any one of claims 10 to 16 wherein the formulation is in the form of a dressing impregnated at least one antisense polynucleotide to an osteopontin.

22. A formulation according to any one of claims 10 to 21, wherein the polynucleotide is DNA.

23. A formulation according to claim 22, wherein the polynucleotide is an oligodeoxynucleotide.

24. A formulation according to claim 23, wherein the oligodeoxynucleotide is an unmodified phosphodiester oligodeoxynucleotide.

25. A formulation according to claim 23, wherein the oligodeoxynucleotide is a chemically modified deoxyoligonucleotide.

26. A formulation according to claim 25, wherein the polynucleotide is a chemically modified oligonucleotide selected from phosphorothioates, methylphosphonates, phosphoramidates, phosphorodithioates, N3′P5′-phosphoramidates, oligoribonucleotide phosphorothioates and their 2′-O-alkyl analogs, 2′-O-methylribonucleotide methylphosphonates, and mixed backbone oligonucleotides.

27. A formulation according to any one of claims 10-26, wherein the polynucleotide is between about 6 and about 40 nucleotides in length.

28. A formulation according to any one of claims 10-27, wherein the polynucleotide is capable of binding to an osteopontin mRNA.

29. A formulation according to claim 28, wherein the polynucleotide has absolute complementarity to the mRNA.

30. A formulation according to claim 28, wherein the polynucleotide has at least about 70% complementarity to the mRNA.

31. A formulation according to claim 28, wherein the polynucleotide may bind to an osteopontin mRNA at either (1) 5′ to the coding sequence, and/or (ii) to the coding sequence, and/or (iii) 3′ to the coding sequence.

32. A method of site-specific downregulation of osteopontin protein expression for a wound-healing purpose which comprises administering a formulation as defined in any one of claims 10 to 31 to a site on or within a patient at which said downregulation is required.

33. Use of the formulation of any of claims 10 to 31 for the manufacture of a medicament for use in the treatment of a wound wherein the formulation is applied prior to repair or closure of a wound.

34. Use of the formulation of claim 33 for the manufacture of a medicament for use in surgery.

35. A method of treating a human subject having a wound, which comprises administering to the wound an osteopontin anti-sense polynucleotide, whereby osteopontin protein expression is down-regulated.

36. A method of treating a human subject to reduce inflammation associated with a wound or associated with a tissue subjected to a physical trauma which comprises the step of administering to the wound or tissue an amount of an osteopontin anti-sense polynucleotide effective to downregulate osteopontin expression.

37. A method of decreasing scar formation following a wound to a human subject which comprises administering to the wound an amount of an osteopontin anti-sense polynucleotide effective to downregulate osteopontin expression.

38. A product for downregulation of osteopontin expression comprising a sustained release formulation suitable for topical application having a concentration of about 0.05 μM to about 50 μM of an oligonucleotide capable of binding to osteopontin mRNA.

39. The product of claim 1 wherein the formulation has an oligonucleotide concentration of about 2 μM or about 5 μM.

40. The product of claim 38 or 39 wherein the oligonucleotide is an oligodeoxynucleotide.

41. The product of claim 40 wherein the oligodeoxynucleotide is single stranded.

42. The product according to any of claims 38 to 40 wherein the osteopontin is human osteopontin.

43. The product according to any of claims 38 to 41 wherein the product is in the form of a gel, liquid or foam.

44. The product according to any of claims 38 to 41 wherein the product is in the form of a dressing.

45. A method of treating a subject having a wound not healing at an expected rate, which comprises administration of an effective amount of an antiosteopontin polynucleotide to the wound.

46. A method according to claim 45 wherein the antiosteopontin polynucleotide is an osteopontin antisense polynucleotide.

47. A method according to claim 45 wherein the antiosteopontin polynucleotide is an RNAi or siRNA polynucleotide.

48. A method according to claim 45 wherein the wound is a dehiscent wound.

49. A method according to claim 45 wherein the wound is a delayed or incompletely healing wound.

50. A method according to claim 45 wherein the wound is a chronic wound.

51. A method according to claim 45 wherein the wound is a vasculitic ulcer, a venous ulcer, a venous stasis ulcer, an arterial ulcer, a pressure ulcer, a decubitus ulcer, or a diabetic ulcer.

52. A method according to any of claims 45 to 51 wherein the subject is a human.

53. A method according to any of claims 45 to 51 wherein the subject is a non-human animal.

54. A method according to claim 53 wherein the animal is a horse, a dog or a cat.

55. A method according to any of claims 45 to 51, wherein the anti-osteopontin polynucleotide is administered in an amount ranging from about 1 to about 100 μg per square centimeter of wound size.

56. A method according to any of claims 45 to 51 wherein the administration of said anti-osteopontin polynucleotide is repeated.

57. A method according to claim 56 wherein the administration of said anti-osteopontin polynucleotide is repeated about once per week, whereby wound healing is promoted.

58. A method of treatment comprising administering to a subject in need thereof a composition comprising therapeutically effective amounts of a first wound-healing agent and a second wound-healing agent, wherein said first agent is an anti-osteopontin polynucleotide and said second agent is selected from the group consisting of an anti-connexin 43 polynucleotide, an anti-connexin 43 peptide or peptidomimetic, a hemichannel closing or blocking agent, and a connexin 43 carboxy-terminal polypeptide gap junction closing or blocking agent.

59. A method according to claim 58, wherein said anti-osteopontin polynucleotide and/or said anti-connexin 43 polynucleotide is an antisense polynucleotide.

60. A method according to claim 58 wherein said first and second wound-healing agents are administered in combination.

61. A method according to claim 58 wherein said first and second wound-healing agents are administered separately at about the same time.

62. A method according to claim 58 wherein said first and second wound-healing agents are administered sequentially.

63. A method according to claim 62 wherein the first wound-healing agent is administered first.

64. A method according to claim 62 wherein said second wound-healing agent is administered first.

65. A method according to claim 63 or 64 wherein said first and second wound-healing agents are administered within about 1-6 hours of each other.

66. A method according to claim 63 or 64 wherein said first and second wound-healing agents are administered within about 6-24 hours of each other.

67. A method according to claim 63 or 64 wherein said first and second wound-healing agents are administered within about 1-2 days of each other.

68. A method according to claim 63 or 64 wherein said first and second wound-healing agents are administered within about 1-7 days of each other.

69. A method according to claim 58 wherein more than one of said second wound-healing agents is administered.

70. A method according to claim 58, wherein the composition comprises about 0.001 to about 1 milligrams of said anti-osteopontin polynucleotide and/or anti-connexin 43 polynucleotide.

71. A method according to claim 58, wherein said anti-osteopontin polynucleotide is an RNAi or siRNA polynucleotide.

72. A method according to claim 58, wherein the subject is a mammal.

73. A method according to claim 72, wherein the mammal is a human.

74. A method according to claim 73, wherein the mammal is selected from the group consisting of domestic animals, farm animals, zoo animals, sports animals, and pets.

75. A method according to claim 74, wherein the mammal is a horse, dog or cat.

76. A method according to claim 58, wherein the subject has an acute wound.

77. A method according to claim 58, wherein the subject has a chronic wound.

78. A method according to claim 77, wherein the chronic wound is a diabetic ulcer, a venous ulcer, a pressure ulcer, a vasculitic ulcer, or an arterial ulcer.

79. A method of treatment comprising administering to a subject in need thereof a first composition and a second composition, said first composition comprising a therapeutically effective amount of a anti-osteopontin polynucleotide and said second composition comprising a therapeutically effective amount of an anti-connexin 43 polynucleotide.

80. A pharmaceutical composition for use in promoting or improving wound healing, which comprises therapeutically effective amounts of an anti-osteopontin polynucleotide and an anti-connexin 43 polynucleotide or an anti-connexin 43 peptide or peptidomimetic.

81. A pharmaceutical composition according to claim 80 which is formulated for topical administration.

82. A pharmaceutical composition according to claim 80 which is formulated as a gel.

83. A method for treating chronic wounds, comprising administering to a subject in need thereof a therapeutically effective amount of a pharmaceutical composition according to claim 80.

84. A method for reducing scar formation in a subject in need thereof, comprising administering to said subject a therapeutically effective amount of a pharmaceutical composition according claim 80.

85. A method of preparing a medicament for treating a wound, comprising bringing together and an amount of a first composition and a second composition, wherein said first composition comprises an effective amount of an anti-osteopontin polynucleotide and said second composition comprises an effective amount of a compound selected from the group consisting of an anti-connexin 43 polynucleotide, an anti-connexin 43 peptide or peptidomimetic, a hemichannel closing agent, and a connexin 43 carboxy-terminal polypeptide gap junction closing agent.

86. A method according to claim 85 wherein either or both of said anti-osteopontin polynucleotide and said anti-connexin 43 polynucleotide comprise an antisense polynucleotide.

87. A method according to claim 85 wherein either or both of said anti-osteopontin polynucleotide and said anti-connexin 43 polynucleotide comprise an RNAi or an siRNA polynucleotide.

88. A method of any of claim 85, 86 or 87 wherein said medicament is formulated for topical administration.

89. A method of claim 85, 86 or 87 wherein said medicament is formulated for sustained release.

90. An article of manufacture comprising package material containing a polynucleotide, formulation, pharmaceutical composition or medicament according to any of the preceding claims together with instructions for use in or on a subject in order to promote or improve wound healing or tissue repair.

91. A wound dressing comprising an anti-osteopontin polynucleotide and/or an anti-connexin 43 polynucleotide.

92. A method of treatment comprising administering to a subject in need thereof a composition comprising therapeutically effective amounts of an anti-osteopontin agent and one or more anti-connexin 43 polynucleotides, anti-connexin 43 peptides, anti-connexin 43 peptidomimetics, gap junction closing compounds, hemichannel closing compounds, or connexin carboxy-terminal polypeptides for the treatment of wounds.

93. A method according to claim 92, wherein said anti-connexin 43 polynucleotide is an connexin 43 antisense polynucleotide.

94. A method according to claim 93, wherein said connexin 43 antisense polynucleotide comprises a sequence selected from SEQ.ID.NOS:1 to 12.

95. A method according to claim 93, wherein said connexin 43 antisense polynucleotide is selected from:
(SEQ ID NO: 1)
GTA ATT GCG GCA AGA AGA ATT GTT TCT GTC;
(SEQ ID NO: 2)
GTA ATT GCG GCA GGA GGA ATT GTT TCT GTC;
and,
(SEQ ID NO: 3)
GGC AAG AGA CAC CAA AGA CAC TAC CAG CAT.

96. A method according to claim 93, wherein said antisense polynucleotide has from about 15 to about 35 nucleotides and is sufficiently complementary to connexin 43 mRNA to form a duplex having a melting point greater than 20° C. under physiological conditions.

97. A method according to claim 93, wherein the antisense polynucleotide has from about 15 to about 35 nucleotides and has at least about 70 percent homology to an antisense sequence of connexin 43 mRNA.

98. A method according to claim 92, wherein the composition comprises about 0.1 to about 1000 micrograms of said anti-connexin agent and the anti-connexin 43 agent is an antisense polynucleotide.

99. A method of claim 92, wherein said peptide comprises a sequence selected from SEQ.ID.NOS:15 to 23.

100. A method according to claim 92, wherein the composition comprises about 0.01 to about 100 milligrams of said anti-connexin 43 peptide or anti-connexin 43 peptidomimetic.

101. A method according to claim 92, wherein said anti-connexin agent is an RNAi or siRNA polynucleotide.

102. A method of claim 92, wherein said anti-osteopontin agent is selected from:

| | |
|---|---|
| CAA TCT CAT GGT CGT AG; | (SEQ.ID.NO: 158) |
| CTA TAG GAT CTG GGT GC, | (SEQ.ID.NO: 159) |
| CAT TGG AAT TGC TTG GA, | (SEQ.ID.NO: 160) |
| CCA CAG AAT CCT CGC TC, | (SEQ.ID.NO: 161) |
| GTT GGG GAC ATC GAC TG, | (SEQ.ID.NO: 162) |
| AGC TAT CAC CTC GGC CG, | (SEQ.ID.NO: 163) |
| or | |
| ACT ATC GAT CAC ATC CG. | (SEQ.ID.NO: 164) |

103. A method according to claim 92, wherein the subject is a mammal.

104. A method according to claim 103, wherein the mammal is a human.

105. A method according to claim 103, wherein the mammal is selected from the group consisting of domestic animals, farm animals, zoo animals, sports animals, and pets.


SEQUENCE LISTING (176 sequences)

SEQ ID NO: 1 - DNA, 30 bp, Artificial Sequence
  source: /note=“Description of Artificial Sequence Artificial AS ODN Cx43 oligonucleotide”
gtaattgcggcaagaagaattgtttctgtc

SEQ ID NO: 2 - DNA, 30 bp, Artificial Sequence
  source: /note=“Description of Artificial Sequence Artificial AS ODN Cx43 oligonucleotide”
gtaattgcggcaggaggaattgtttctgtc

SEQ ID NO: 3 - DNA, 30 bp, Artificial Sequence
  source: /note=“Description of Artificial Sequence Artificial AS ODN Cx43 oligonucleotide”
ggcaagagacaccaaagacactaccagcat

SEQ ID NO: 4 - DNA, 27 bp, Artificial Sequence
  source: /note=“Description of Artificial Sequence Artificial AS ODN Cx26 oligonucleotide”
tcctgagcaatacctaacgaacaaata

SEQ ID NO: 5 - DNA, 20 bp, Artificial Sequence
  source: /note=“Description of Artificial Sequence Artificial AS ODN Cx37 oligonucleotide”
catctccttggtgctcaacc

SEQ ID NO: 6 - DNA, 20 bp, Artificial Sequence
  source: /note=“Description of Artificial Sequence Artificial AS ODN Cx37 oligonucleotide”
ctgaagtcgacttggcttgg

SEQ ID NO: 7 - DNA, 21 bp, Artificial Sequence
  source: /note=“Description of Artificial Sequence Artificial AS ODN Cx30 oligonucleotide”
ctcagatagtggccagaatgc

SEQ ID NO: 8 - DNA, 20 bp, Artificial Sequence
  source: /note=“Description of Artificial Sequence Artificial AS ODN Cx30 oligonucleotide”
ttgtccaggtgactccaagg

SEQ ID NO: 9 - DNA, 25 bp, Artificial Sequence
  source: /note=“Description of Artificial Sequence Artificial AS ODN Cx31.1 oligonucleotide”
cgtccgagcccagaaagatgaggtc

SEQ ID NO: 10 - DNA, 19 bp, Artificial Sequence
  source: /note=“Description of Artificial Sequence Artificial AS ODN Cx31.1 oligonucleotide”
agaggcgcacgtgagacac

SEQ ID NO: 11 - DNA, 19 bp, Artificial Sequence
  source: /note=“Description of Artificial Sequence Artificial AS ODN Cx31.1 oligonucleotide”
tgaagacaatgaagatgtt

SEQ ID NO: 12 - DNA, 25 bp, Artificial Sequence
  source: /note=“Description of Artificial Sequence Artificial AS ODN Cx32 oligonucleotide”
tttcttttctatgtgctgttggtga

SEQ ID NO: 13 - protein, 396 aa, Homo sapiens
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDNEEDPMMYPEMELESDKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKVGSKAGSNKSTASSKSGDGKNSVWI

SEQ ID NO: 14 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 15 - protein, 11 aa, Artificial Sequence
  source: /note=“Description of Artificial Sequence Artificial Cx43 Cx/anti-Cx P peptide”
FEVAFLLIQWI

SEQ ID NO: 16 - protein, 11 aa, Artificial Sequence
  source: /note=“Description of Artificial Sequence Artificial Cx43 Cx/anti-Cx P peptide”
LLIQWYIGFSL

SEQ ID NO: 17 - protein, 16 aa, Artificial Sequence
  source: /note=“Description of Artificial Sequence Artificial Cx43 Cx/anti-Cx P peptide”
SLSAVYTCKRDPCPHQ

SEQ ID NO: 18 - protein, 12 aa, Artificial Sequence
  source: /note=“Description of Artificial Sequence Artificial Cx43 Cx/anti-Cx P peptide”
VDCFLSRPTEKT

SEQ ID NO: 19 - protein, 11 aa, Artificial Sequence
  source: /note=“Description of Artificial Sequence Artificial Cx43 Cx/anti-Cx P peptide”
SRPTEKTIFII

SEQ ID NO: 20 - protein, 13 aa, Artificial Sequence
  source: /note=“Description of Artificial Sequence Artificial Cx43 Cx/anti-Cx P peptide”
LGTAVESAWGDEQ

SEQ ID NO: 21 - protein, 12 aa, Artificial Sequence
  source: /note=“Description of Artificial Sequence Artificial Cx43 Cx/anti-Cx P peptide”
QSAFRCNTQQPG

SEQ ID NO: 22 - protein, 12 aa, Artificial Sequence
  source: /note=“Description of Artificial Sequence Artificial Cx43 Cx/anti-Cx P peptide”
QQPGCENVCYDK

SEQ ID NO: 23 - protein, 13 aa, Artificial Sequence
  source: /note=“Description of Artificial Sequence Artificial Cx43 Cx/anti-Cx P peptide”
VCYDKSFPISHVR

SEQ ID NO: 24 - protein, 38 aa, Artificial Sequence
  source: /note=“Description of Artificial Sequence Artificial Cx32 Cx/anti-Cx P polypeptide”
AAESVWGDEIKSSFICNTLQPGCNSVCYDHFFPISHVR

SEQ ID NO: 25 - protein, 12 aa, Artificial Sequence
  source: /note=“Description of Artificial Sequence Artificial Cx32 Cx/anti-Cx P peptide”
ESVWGDEKSSFI

SEQ ID NO: 26 - protein, 12 aa, Artificial Sequence
  source: /note=“Description of Artificial Sequence Artificial Cx32 Cx/anti-Cx P peptide”
ICNTLQPGCNSV

SEQ ID NO: 27 - protein, 12 aa, Artificial Sequence
  source: /note=“Description of Artificial Sequence Artificial Cx32 Cx/anti-Cx P peptide”
SVCYDHFFPISH

SEQ ID NO: 28 - protein, 25 aa, Artificial Sequence
  source: /note=“Description of Artificial Sequence Artificial Cx32 Cx/anti-Cx P peptide”
RLVKCEAFPCPNTVDCFVSRPTEKT

SEQ ID NO: 29 - protein, 12 aa, Artificial Sequence
  source: /note=“Description of Artificial Sequence Artificial Cx32 Cx/anti-Cx P peptide”
VKCEAFPCPNTV

SEQ ID NO: 30 - protein, 12 aa, Artificial Sequence
  source: /note=“Description of Artificial Sequence Artificial Cx32 Cx/anti-Cx P peptide”
VDCFVSRPTEKT

SEQ ID NO: 31 - protein, 13 aa, Artificial Sequence
  source: /note=“Description of Artificial Sequence Artificial Cx32 Cx/anti-Cx P peptide”
VCYDHFFPISHVR

SEQ ID NO: 32 - protein, 18 aa, Artificial Sequence
  source: /note=“Description of Artificial Sequence Artificial Cx32 Cx/anti-Cx P peptide”
VWGDEKSSFICNTLQPGY

SEQ ID NO: 33 - protein, 15 aa, Artificial Sequence
  source: /note=“Description of Artificial Sequence Artificial Cx32 Cx/anti-Cx P peptide”
DEKSSFICNTLQPGY

SEQ ID NO: 34 - protein, 11 aa, Artificial Sequence
  source: /note=“Description of Artificial Sequence Artificial Cx32 Cx/anti-Cx P peptide”
SRPTEKTVFTV

SEQ ID NO: 35 - protein, 7 aa, Artificial Sequence
  source: /note=“Description of Artificial Sequence Artificial Cx32/Cx43 Cx/anti-Cx P peptide”
SRPTEKT

SEQ ID NO: 36 - protein, 12 aa, Artificial Sequence
  source: /note=“Description of Artificial Sequence Artificial Cx32 Cx/anti-Cx P peptide”
ICNTLQPGCNSV

SEQ ID NO: 37 - protein, 16 aa, Artificial Sequence
  source: /note=“Description of Artificial Sequence Artificial Cx40 Cx/anti-Cx P peptide”
FLDTLHVCRRSPCPHP

SEQ ID NO: 38 - protein, 18 aa, Artificial Sequence
  source: /note=“Description of Artificial Sequence Artificial Cx43 Cx/anti-Cx P peptide”
KRDPCHQVDCFLSRPTEK

SEQ ID NO: 39 - protein, 35 aa, Artificial Sequence
  source: /note=“Description of Artificial Sequence Artificial huCx26 EC domain polypeptide”
KEVWGDEQADFVCNTLQPGCKNVCYDHYFPISHIR

SEQ ID NO: 40 - protein, 35 aa, Artificial Sequence
  source: /note=“Description of Artificial Sequence Artificial huCx30 EC domain polypeptide”
QEVWGDEQEDFVCNTLQPGCKNVCYDHFFPVSHIR

SEQ ID NO: 41 - protein, 35 aa, Artificial Sequence
  source: /note=“Description of Artificial Sequence Artificial huCx30.3 EC domain polypeptide”
EEVWDDEQKDFVCNTKQPGCPNVCYDEFFPVSHVR

SEQ ID NO: 42 - protein, 35 aa, Artificial Sequence
  source: /note=“Description of Artificial Sequence Artificial huCx31 EC domain polypeptide”
ERVWGDEQKDFDCNTKQPGCTNVCYDNYFPISNIR

SEQ ID NO: 43 - protein, 35 aa, Artificial Sequence
  source: /note=“Description of Artificial Sequence Artificial huCx31.1 EC domain polypeptide”
ERVWSDDHKDFDCNTRQPGCSNVCFDEFFPVSHVR

SEQ ID NO: 44 - protein, 35 aa, Artificial Sequence
  source: /note=“Description of Artificial Sequence Artificial huCx32 EC domain polypeptide”
ESVWGDEKSSFICNTLQPGCNSVCYDQFFPISHVR

SEQ ID NO: 45 - protein, 35 aa, Artificial Sequence
  source: /note=“Description of Artificial Sequence Artificial huCx36 EC domain polypeptide”
ESVWGDEQSDFECNTAQPGCTNVCYDQAFPISHIR

SEQ ID NO: 46 - protein, 35 aa, Artificial Sequence; identical to SEQ ID NO: 45
  source: /note=“Description of Artificial Sequence Artificial huCx37 EC domain polypeptide”

SEQ ID NO: 47 - protein, 35 aa, Artificial Sequence
  source: /note=“Description of Artificial Sequence Artificial huCx40.1 EC domain polypeptide”
RPVYQDEQERFVCNTLQPGCANVCYDVFSPVSHLR

SEQ ID NO: 48 - protein, 35 aa, Artificial Sequence
  source: /note=“Description of Artificial Sequence Artificial huCx43 EC domain polypeptide”
ESAWGDEQSAFRCNTQQPGCENVCYDKSFPISHVR

SEQ ID NO: 49 - protein, 35 aa, Artificial Sequence
  source: /note=“Description of Artificial Sequence Artificial huCx46 EC domain polypeptide”
EDVWGDEQSDFTCNTQQPGCBNVCYBRAFPISHIR

SEQ ID NO: 50 - protein, 35 aa, Artificial Sequence
  source: /note=“Description of Artificial Sequence Artificial huCx46.6 EC domain polypeptide”
EAIYSDEQAKFTCNTRQPGCDNVCYDAFAPLSHVR

SEQ ID NO: 51 - protein, 35 aa, Artificial Sequence
  source: /note=“Description of Artificial Sequence Artificial huCx40 EC domain polypeptide”
ESSWGDEQADFRCDTIQPGCQNVCTDQAFPISHIR

SEQ ID NO: 52 - protein, 36 aa, Artificial Sequence
  source: /note=“Description of Artificial Sequence Artificial huCx45 EC domain polypeptide”
GESIYYDEQSKFVCNTEQPGCENVCYDAFAPLSHVR

SEQ ID NO: 53 - protein, 39 aa, Artificial Sequence
  source: /note=“Description of Artificial Sequence Artificial huCx26 EC domain polypeptide”
MYVFYVMYDGFSMQRLVKCNAWPCPNTVDCFVSRPTEKT

SEQ ID NO: 54 - protein, 39 aa, Artificial Sequence
  source: /note=“Description of Artificial Sequence Artificial huCx30 EC domain polypeptide”
MYVFYFLYNGYHLPWVLKCGIDPCPNLVDCFISRPTEKT

SEQ ID NO: 55 - protein, 39 aa, Artificial Sequence
  source: /note=“Description of Artificial Sequence Artificial huCx30.3 EC domain polypeptide”
LYIFHRLYKDYDMPRVVACSVEPCPHTVDCYISRPTEKK

SEQ ID NO: 56 - protein, 40 aa, Artificial Sequence
  source: /note=“Description of Artificial Sequence Artificial huCx31 EC domain polypeptide”
LYLLHTLWHGFNMPRLVQCANVAPCPNIVDCYIARPTEKK

SEQ ID NO: 57 - protein, 39 aa, Artificial Sequence
  source: /note=“Description of Artificial Sequence Artificial huCx31.1 EC domain polypeptide”
LYVFHSFYPKYILPPVVKCHADPCPNIVDCFISKPSEKN

SEQ ID NO: 58 - protein, 39 aa, Artificial Sequence
  source: /note=“Description of Artificial Sequence Artificial huCx32 EC domain polypeptide”
MYVFYLLYPGYAMVRLVKCDVYPCPNTVDCFVSRPTEKT

SEQ ID NO: 59 - protein, 32 aa, Artificial Sequence
  source: /note=“Description of Artificial Sequence Artificial huCx36 EC domain polypeptide”
LYGWTMEPVFVCQRAPCPYLVDCFVSRPTEKT

SEQ ID NO: 60 - protein, 32 aa, Artificial Sequence; identical to SEQ ID NO: 59
  source: /note=“Description of Artificial Sequence Artificial huCx37 EC domain polypeptide”

SEQ ID NO: 61 - protein, 38 aa, Artificial Sequence
  source: /note=“Description of Artificial Sequence Artificial huCx40.1 EC domain polypeptide”
GALHYFLFGFLAPKKFPCTRPPCTGVVDCYVSRPTSKS

SEQ ID NO: 62 - protein, 38 aa, Artificial Sequence
  source: /note=“Description of Artificial Sequence Artificial huCx43 EC domain polypeptide”
LLIQWYIYGFSLSAVYTCKRDPCPHQVDCFLSRPTEKT

SEQ ID NO: 63 - protein, 38 aa, Artificial Sequence
  source: /note=“Description of Artificial Sequence Artificial huCx46 EC domain polypeptide”
IAGQYFLYGFELKPLYRCDRWPCPNTVDCFISRPTEKT

SEQ ID NO: 64 - protein, 38 aa, Artificial Sequence
  source: /note=“Description of Artificial Sequence Artificial huCx46.6 EC domain polypeptide”
LVGQYLLYGFEVRPFFPCSRQPCPHVVDCFVSRPTEKT

SEQ ID NO: 65 - protein, 38 aa, Artificial Sequence
  source: /note=“Description of Artificial Sequence Artificial huCx40 EC domain polypeptide”
IVGQYFIYGIFLTTLHVCRRSPCPHPVNCYVSRPTEKN

SEQ ID NO: 66 - protein, 38 aa, Artificial Sequence
  source: /note=“Description of Artificial Sequence Artificial huCx45 EC domain polypeptide”
LIGQYFLYGFQVHPFYVCSRLPCHPKIDCFISRPTEKT

SEQ ID NO: 67 - protein, 42 aa, Artificial Sequence
  source: /note=“Description of Artificial Sequence Artificial huCx43 EC domain polypeptide”
LLIQWYIYGFSLSAVYTCKRDPCPHQVDCFLSRPTEKTIFII

SEQ ID NO: 68 - protein, 43 aa, Artificial Sequence
  source: /note=“Description of Artificial Sequence Artificial huCx26 EC domain polypeptide”
MYVFYVMYDGFSMQRLVKCNAWPCPNTVDCFVSRPTEKTVFTV

SEQ ID NO: 69 - protein, 42 aa, Artificial Sequence
  source: /note=“Description of Artificial Sequence Artificial huCx30 EC domain polypeptide”
YVFYFLYNGYHLPWVLKCGIDPCPNLVDCFISRPTEKTVFTI

SEQ ID NO: 70 - protein, 43 aa, Artificial Sequence
  source: /note=“Description of Artificial Sequence Artificial huCx30.3 EC domain polypeptide”
LYIFHRLYKDYDMPRVVACSVEPCPHTVDCYISRPTEKKVFTY

SEQ ID NO: 71 - protein, 42 aa, Artificial Sequence
  source: /note=“Description of Artificial Sequence Artificial huCx31 EC domain polypeptide”
LYLLHTLWHGFNMPRLVQCANVAPCPNIVDCYIARPTEKKTY

SEQ ID NO: 72 - protein, 43 aa, Artificial Sequence
  source: /note=“Description of Artificial Sequence Artificial huCx31.1 EC domain polypeptide”
LYVFHSFYPKYILPPVVKCHADPCPNIVDCFISKPSEKNIFTL

SEQ ID NO: 73 - protein, 43 aa, Artificial Sequence
  source: /note=“Description of Artificial Sequence Artificial huCx32 EC domain polypeptide”
MYVFYLLYPGYAMVRLVKCDVYPCPNTVDCFVSRPTEKTVFTV

SEQ ID NO: 74 - protein, 36 aa, Artificial Sequence
  source: /note=“Description of Artificial Sequence Artificial huCx36 EC domain polypeptide”
LYGWTMEPVFVCQRAPCPYLVDCFVSRPTEKTIFII

SEQ ID NO: 75 - protein, 36 aa, Artificial Sequence; identical to SEQ ID NO: 74
  source: /note=“Description of Artificial Sequence Artificial huCx37 EC domain polypeptide”

SEQ ID NO: 76 - protein, 42 aa, Artificial Sequence
  source: /note=“Description of Artificial Sequence Artificial huCx40.1 EC domain polypeptide”
GALHYFLFGFLAPKKFPCTRPPCTGVVDCYVSRPTEKSLLML

SEQ ID NO: 77 - protein, 42 aa, Artificial Sequence
  source: /note=“Description of Artificial Sequence Artificial huCx46 EC domain polypeptide”
IAGQYFLYGFELKPLYRCDRWPCPNTVDCFISRPTEKTIFII

SEQ ID NO: 78 - protein, 42 aa, Artificial Sequence
  source: /note=“Description of Artificial Sequence Artificial huCx46.1 EC domain polypeptide”
LVGQYLLYGFEVRPFFPCSRQPCPHVVDCFVSRPTEKTVFLL

SEQ ID NO: 79 - protein, 41 aa, Artificial Sequence
  source: /note=“Description of Artificial Sequence Artificial huCx40 EC domain polypeptide”
IVGQYFIYGIFLTTLHVCRRSPCPHPVNCYSRPTEKNVFIV

SEQ ID NO: 80 - protein, 42 aa, Artificial Sequence
  source: /note=“Description of Artificial Sequence Artificial huCx45 EC domain polypeptide”
LIGQYFLYGFQVHPFYVCSRLPCHPKIDCFISRPTEKTIFLL

SEQ ID NO: 81 - protein, 45 aa, Artificial Sequence
  source: /note=“Description of Artificial Sequence Artificial Cx40 Cx/anti-Cx P polypeptide”
LGTAAESSWGDEQADFRCDTIQPGCQNVCTDQAFPISHIRFWVLQ

SEQ ID NO: 82 - protein, 14 aa, Artificial Sequence
  source: /note=“Description of Artificial Sequence Artificial Cx40 Cx/anti-Cx P peptide”
LGTAAESSWGDEQA

SEQ ID NO: 83 - protein, 13 aa, Artificial Sequence
  source: /note=“Description of Artificial Sequence Artificial Cx40 Cx/anti-Cx P peptide”
DEQADFRCDTIQP

SEQ ID NO: 84 - protein, 13 aa, Artificial Sequence
  source: /note=“Description of Artificial Sequence Artificial Cx40 Cx/anti-Cx P peptide”
TIQPGCQNVCTDQ

SEQ ID NO: 85 - protein, 13 aa, Artificial Sequence
  source: /note=“Description of Artificial Sequence Artificial Cx40 Cx/anti-Cx P peptide”
VCTDQAFPISHIR

SEQ ID NO: 86 - protein, 13 aa, Artificial Sequence
  source: /note=“Description of Artificial Sequence Artificial Cx40 Cx/anti-Cx P peptide”
AFPISHIRFWVLQ

SEQ ID NO: 87 - protein, 47 aa, Artificial Sequence
  source: /note=“Description of Artificial Sequence Artificial Cx40 Cx/anti-Cx P polypeptide”
MEVGFIVGQYFIYGIFLTTLHVCRRSPCPHPVNCYVSRPTEKNVFIV

SEQ ID NO: 88 - protein, 11 aa, Artificial Sequence
  source: /note=“Description of Artificial Sequence Artificial Cx40 Cx/anti-Cx P peptide”
MEVGFIVGQYF

SEQ ID NO: 89 - protein, 12 aa, Artificial Sequence
  source: /note=“Description of Artificial Sequence Artificial Cx40 Cx/anti-Cx P peptide”
IVGQYFIYGIFL

SEQ ID NO: 90 - protein, 14 aa, Artificial Sequence
  source: /note=“Description of Artificial Sequence Artificial Cx40 Cx/anti-Cx P peptide”
GIFLTTLHVCRRSP

SEQ ID NO: 91 - protein, 12 aa, Artificial Sequence
  source: /note=“Description of Artificial Sequence Artificial Cx40 Cx/anti-Cx P peptide”
RRSPCPHPVNCY

SEQ ID NO: 92 - protein, 12 aa, Artificial Sequence
  source: /note=“Description of Artificial Sequence Artificial Cx40 Cx/anti-Cx P peptide”
VNCYVSRPTEKN

SEQ ID NO: 93 - protein, 11 aa, Artificial Sequence
  source: /note=“Description of Artificial Sequence Artificial Cx40 Cx/anti-Cx P peptide”
SRPTEKNVFIV

SEQ ID NO: 94 - protein, 46 aa, Artificial Sequence
  source: /note=“Description of Artificial Sequence Artificial Cx45 Cx/anti-Cx P polypeptide”
LTAVGGESIYYDEQSKFVCNTEQPGCENVCYDAFAPLSHVRFWVFQ

SEQ ID NO: 95 - protein, 15 aa, Artificial Sequence
  source: /note=“Description of Artificial Sequence Artificial Cx45 Cx/anti-Cx P peptide”
LTAVGGESIYYDEQS

SEQ ID NO: 96 - protein, 13 aa, Artificial Sequence
  source: /note=“Description of Artificial Sequence Artificial Cx45 Cx/anti-Cx P peptide”
DEQSKFVCNTEQP

SEQ ID NO: 97 - protein, 13 aa, Artificial Sequence
  source: /note=“Description of Artificial Sequence Artificial Cx45 Cx/anti-Cx P peptide”
TEQPGCENVCYDA

SEQ ID NO: 98 - protein, 13 aa, Artificial Sequence
  source: /note=“Description of Artificial Sequence Artificial Cx45 Cx/anti-Cx P peptide”
VCYDAFAPLSHVR

SEQ ID NO: 99 - protein, 12 aa, Artificial Sequence
  source: /note=“Description of Artificial Sequence Artificial Cx45 Cx/anti-Cx P peptide”
APLSHVRFWVFQ

SEQ ID NO: 100 - protein, 47 aa, Artificial Sequence
  source: /note=“Description of Artificial Sequence Artificial Cx45 Cx/anti-Cx P polypeptide”
FEVGFLIGQYFLYGFQVHPFYVCSRLPCHPKIDCFISRPTEKTIFLL

SEQ ID NO: 101 - protein, 11 aa, Artificial Sequence
  source: /note=“Description of Artificial Sequence Artificial Cx45 Cx/anti-Cx P peptide”
FEVGFLIGQYF

SEQ ID NO: 102 - protein, 12 aa, Artificial Sequence
  source: /note=“Description of Artificial Sequence Artificial Cx45 Cx/anti-Cx P peptide”
LIGQYFLYGFQV

SEQ ID NO: 103 - protein, 14 aa, Artificial Sequence
  source: /note=“Description of Artificial Sequence Artificial Cx45 Cx/anti-Cx P peptide”
GFQVHPFYVCSRLP

SEQ ID NO: 104 - protein, 12 aa, Artificial Sequence
  source: /note=“Description of Artificial Sequence Artificial Cx45 Cx/anti-Cx P peptide”
SRLPCHPKIDCF

SEQ ID NO: 105 - protein, 12 aa, Artificial Sequence
  source: /note=“Description of Artificial Sequence Artificial Cx45 Cx/anti-Cx P peptide”
IDCFISRPTEKT

SEQ ID NO: 106 - protein, 11 aa, Artificial Sequence
  source: /note=“Description of Artificial Sequence Artificial Cx45 Cx/anti-Cx P peptide”
SRPTEKTIFLL

SEQ ID NO: 107 - protein, 11 aa, Artificial Sequence
  source: /note=“Description of Artificial Sequence Artificial Cx/anti-Cx Control P peptide”
SRGGEKNVFIV

SEQ ID NO: 108 - protein, 10 aa, Artificial Sequence
  source: /note=“Description of Artificial Sequence Artificial Cx45 Cx/anti-Cx P peptide”
YVCSRLPCHP

SEQ ID NO: 109 - protein, 11 aa, Artificial Sequence
  source: /note=“Description of Artificial Sequence Artificial Cx45 Cx/anti-Cx P peptide”
QVHPFYVCSRL

SEQ ID NO: 110 - protein, 13 aa, Artificial Sequence
  source: /note=“Description of Artificial Sequence Artificial Cx45 Cx/anti-Cx P peptide”
FEVGFLIGQYFLY

SEQ ID NO: 111 - protein, 12 aa, Artificial Sequence
  source: /note=“Description of Artificial Sequence Artificial Cx45 Cx/anti-Cx P peptide”
GQYFLYGFQVHP

SEQ ID NO: 112 - protein, 12 aa, Artificial Sequence
  source: /note=“Description of Artificial Sequence Artificial Cx45 Cx/anti-Cx P peptide”
GFQVHPFYVCSR

SEQ ID NO: 113 - protein, 12 aa, Artificial Sequence
  source: /note=“Description of Artificial Sequence Artificial Cx45 Cx/anti-Cx P peptide”
AVGGESIYYDEQ

SEQ ID NO: 114 - protein, 12 aa, Artificial Sequence
  source: /note=“Description of Artificial Sequence Artificial Cx45 Cx/anti-Cx P peptide”
YDEQSKFVCNTE

SEQ ID NO: 115 - protein, 12 aa, Artificial Sequence
  source: /note=“Description of Artificial Sequence Artificial Cx45 Cx/anti-Cx P peptide”
NTEQPGCENVCY

SEQ ID NO: 116 - protein, 12 aa, Artificial Sequence
  source: /note=“Description of Artificial Sequence Artificial Cx45 Cx/anti-Cx P peptide”
CYDAFAPLSHVR

SEQ ID NO: 117 - protein, 12 aa, Artificial Sequence
  source: /note=“Description of Artificial Sequence Artificial Cx45 Cx/anti-Cx P peptide”
FAPLSHVRFWVF

SEQ ID NO: 118 - protein, 5 aa, Artificial Sequence
  source: /note=“Description of Artificial Sequence Artificial Cx45 Cx/anti-Cx P peptide”
LIGQY

SEQ ID NO: 119 - protein, 5 aa, Artificial Sequence
  source: /note=“Description of Artificial Sequence Artificial Cx45 Cx/anti-Cx P peptide”
QVHPF

SEQ ID NO: 120 - protein, 5 aa, Artificial Sequence
  source: /note=“Description of Artificial Sequence Artificial Cx45 Cx/anti-Cx P peptide”
YVCSR

SEQ ID NO: 121 - protein, 5 aa, Artificial Sequence
  source: /note=“Description of Artificial Sequence Artificial Cx45 Cx/anti-Cx P peptide”
SRLPC

SEQ ID NO: 122 - protein, 5 aa, Artificial Sequence
  source: /note=“Description of Artificial Sequence Artificial Cx45 Cx/anti-Cx P peptide”
LPCHP

SEQ ID NO: 123 - protein, 5 aa, Artificial Sequence
  source: /note=“Description of Artificial Sequence Artificial Cx45 Cx/anti-Cx P peptide”
GESIY

SEQ ID NO: 124 - protein, 6 aa, Artificial Sequence
  source: /note=“Description of Artificial Sequence Artificial Cx45 Cx/anti-Cx P peptide”
YDEQSK

SEQ ID NO: 125 - protein, 6 aa, Artificial Sequence
  source: /note=“Description of Artificial Sequence Artificial Cx45 Cx/anti-Cx P peptide”
SKFVCN

SEQ ID NO: 126 - protein, 8 aa, Artificial Sequence
  source: /note=“Description of Artificial Sequence Artificial Cx45 Cx/anti-Cx P peptide”
TEQPGCEN

SEQ ID NO: 127 - protein, 8 aa, Artificial Sequence
  source: /note=“Description of Artificial Sequence Artificial Cx45 Cx/anti-Cx P peptide”
VCYDAFAP

SEQ ID NO: 128 - protein, 10 aa, Artificial Sequence
  source: /note=“Description of Artificial Sequence Artificial Cx45 Cx/anti-Cx P peptide”
LSHVRFWVFQ

SEQ ID NO: 129 - protein, 14 aa, Artificial Sequence
  source: /note=“Description of Artificial Sequence Artificial Cx45 Cx/anti-Cx P peptide”
LIQYFLYGFQVHPF

SEQ ID NO: 130 - protein, 13 aa, Artificial Sequence
  source: /note=“Description of Artificial Sequence Artificial Cx45 Cx/anti-Cx P peptide”
VHPFYCSRLPCHP

SEQ ID NO: 131 - protein, 22 aa, Artificial Sequence
  source: /note=“Description of Artificial Sequence Artificial Cx45 Cx/anti-Cx P peptide”
VGGESIYYDEQSKFVCNTEQPG

SEQ ID NO: 132 - protein, 22 aa, Artificial Sequence
  source: /note=“Description of Artificial Sequence Artificial Cx45 Cx/anti-Cx P peptide”
TEQPGCENVCYDAFAPLSHVRF

SEQ ID NO: 133 - protein, 14 aa, Artificial Sequence
  source: /note=“Description of Artificial Sequence Artificial Cx45 Cx/anti-Cx P peptide”
AFAPLSHVRFWVFQ

SEQ ID NO: 134 - DNA, 1314 bp, Homo sapiens
ggcttttagcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 135 - DNA, 1149 bp, Homo sapiens
atgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 136 - DNA, 15 bp, Artificial Sequence
  source: /note=“Description of Artificial Sequence Artificial dnazyme Cat site oligonucleotide”
ggctagctacaacga

SEQ ID NO: 137 - DNA, 31 bp, Artificial Sequence
  source: /note=“Description of Artificial Sequence Artificial dnazyme osteopontin oligonucleotide”
caatctcaggctagctacaacgaggtcgtag

SEQ ID NO: 138 - DNA, 31 bp, Artificial Sequence
  source: /note=“Description of Artificial Sequence Artificial dnazyme osteopontin oligonucleotide”
ctataggaggctagctacaacgactgggtgc

SEQ ID NO: 139 - DNA, 31 bp, Artificial Sequence
  source: /note=“Description of Artificial Sequence Artificial dnazyme osteopontin oligonucleotide”
cattggaaggctagctacaacgatgcttgga

SEQ ID NO: 140 - DNA, 31 bp, Artificial Sequence
  source: /note=“Description of Artificial Sequence Artificial dnazyme osteopontin oligonucleotide”
ccacagaaggctagctacaacgacctcgctc

SEQ ID NO: 141 - DNA, 31 bp, Artificial Sequence
  source: /note=“Description of Artificial Sequence Artificial dnazyme osteopontin oligonucleotide”
tggtgagaggctagctacaacgatcgtcaga

SEQ ID NO: 142 - DNA, 31 bp, Artificial Sequence
  source: /note=“Description of Artificial Sequence Artificial dnazyme osteopontin oligonucleotide”
catccgaaggctagctacaacgaggtgagat

SEQ ID NO: 143 - DNA, 31 bp, Artificial Sequence
  source: /note=“Description of Artificial Sequence Artificial dnazyme osteopontin oligonucleotide”
agcagtgaggctagctacaacgaggtctcat

SEQ ID NO: 144 - DNA, 31 bp, Artificial Sequence
  source: /note=“Description of Artificial Sequence Artificial dnazyme osteopontin oligonucleotide”
cttgtgtaggctagctacaacgatagcagtg

SEQ ID NO: 145 - DNA, 31 bp, Artificial Sequence
  source: /note=“Description of Artificial Sequence Artificial dnazyme osteopontin oligonucleotide”
gacatcgaggctagctacaacgatgtaggga

SEQ ID NO: 146 - DNA, 31 bp, Artificial Sequence
  source: /note=“Description of Artificial Sequence Artificial dnazyme osteopontin oligonucleotide”
gttggggaggctagctacaacgaatcgactg

SEQ ID NO: 147 - DNA, 31 bp, Artificial Sequence
  source: /note=“Description of Artificial Sequence Artificial dnazyme osteopontin oligonucleotide”
agctatcaggctagctacaacgactcggccg

SEQ ID NO: 148 - DNA, 31 bp, Artificial Sequence
  source: /note=“Description of Artificial Sequence Artificial dnazyme osteopontin oligonucleotide”
ccaagctaggctagctacaacgacacctcgg

SEQ ID NO: 149 - DNA, 31 bp, Artificial Sequence
  source: /note=“Description of Artificial Sequence Artificial dnazyme osteopontin oligonucleotide”
tagactcaggctagctacaacgacgctcttc

SEQ ID NO: 150 - DNA, 31 bp, Artificial Sequence
  source: /note=“Description of Artificial Sequence Artificial dnazyme osteopontin oligonucleotide”
tccttagaggctagctacaacgatcaccgct

SEQ ID NO: 151 - DNA, 31 bp, Artificial Sequence
  source: /note=“Description of Artificial Sequence Artificial dnazyme osteopontin oligonucleotide”
ctttggaaggctagctacaacgagctcaagt

SEQ ID NO: 152 - DNA, 31 bp, Artificial Sequence
  source: /note=“Description of Artificial Sequence Artificial dnazyme osteopontin oligonucleotide”
cgatcacaggctagctacaacgaccgactga

SEQ ID NO: 153 - DNA, 31 bp, Artificial Sequence
  source: /note=“Description of Artificial Sequence Artificial dnazyme osteopontin oligonucleotide”
actatcgaggctagctacaacgacacatccg

SEQ ID NO: 154 - DNA, 31 bp, Artificial Sequence
  source: /note=“Description of Artificial Sequence Artificial dnazyme osteopontin oligonucleotide”
cttgactaggctagctacaacgacgatcaca

SEQ ID NO: 155 - DNA, 31 bp, Artificial Sequence
  source: /note=“Description of Artificial Sequence Artificial dnazyme osteopontin oligonucleotide”
ttgcttgaggctagctacaacgatatcgatc

SEQ ID NO: 156 - DNA, 31 bp, Artificial Sequence
  source: /note=“Description of Artificial Sequence Artificial dnazyme osteopontin oligonucleotide”
ctttggaaggctagctacaacgattgcttga

SEQ ID NO: 157 - DNA, 31 bp, Artificial Sequence
  source: /note=“Description of Artificial Sequence Artificial dnazyme osteopontin oligonucleotide”
attcggaaggctagctacaacgattcagata

SEQ ID NO: 158 - DNA, 17 bp, Artificial Sequence
  source: /note=“Description of Artificial Sequence Artificial AS osteopontin oligonucleotide”
caatctcatggtcgtag

SEQ ID NO: 159 - DNA, 17 bp, Artificial Sequence
  source: /note=“Description of Artificial Sequence Artificial AS osteopontin oligonucleotide”
ctataggatctgggtgc

SEQ ID NO: 160 - DNA, 17 bp, Artificial Sequence
  source: /note=“Description of Artificial Sequence Artificial AS osteopontin oligonucleotide”
cattggaattgcttgga

SEQ ID NO: 161 - DNA, 17 bp, Artificial Sequence
  source: /note=“Description of Artificial Sequence Artificial AS osteopontin oligonucleotide”
ccacagaatcctcgctc

SEQ ID NO: 162 - DNA, 17 bp, Artificial Sequence
  source: /note=“Description of Artificial Sequence Artificial AS osteopontin oligonucleotide”
gttggggacatcgactg

SEQ ID NO: 163 - DNA, 17 bp, Artificial Sequence
  source: /note=“Description of Artificial Sequence Artificial AS osteopontin oligonucleotide”
agctatcacctcggccg

SEQ ID NO: 164 - DNA, 17 bp, Artificial Sequence
  source: /note=“Description of Artificial Sequence Artificial AS osteopontin oligonucleotide”
actatcgatcacatccg

SEQ ID NO: 165 - DNA, 17 bp, Artificial Sequence
  source: /note=“Description of Artificial Sequence Artificial control osteopontin oligonucleotide”
ctacgaccatgagattg

SEQ ID NO: 166 - DNA, 17 bp, Artificial Sequence
  source: /note=“Description of Artificial Sequence Artificial      control osteopontin oligonucleotide”
gcacccagatcctatag

SEQ ID NO: 167 - DNA, 17 bp, Artificial Sequence
  source: /note=“Description of Artificial Sequence Artificial control osteopontin oligonucleotide”
tccaagcaattccaatg

SEQ ID NO: 168 - DNA, 17 bp, Artificial Sequence
  source: /note=“Description of Artificial Sequence Artificial control osteopontin oligonucleotide”
gagcgaggattctgtgg

SEQ ID NO: 169 - DNA, 17 bp, Artificial Sequence
  source: /note=“Description of Artificial Sequence Artificial control osteopontin oligonucleotide”
cagtcgatgtccccaac

SEQ ID NO: 170 - DNA, 17 bp, Artificial Sequence
  source: /note=“Description of Artificial Sequence Artificial control osteopontin oligonucleotide”
cggccgaggtgatagct

SEQ ID NO: 171 - DNA, 17 bp, Artificial Sequence
  source: /note=“Description of Artificial Sequence Artificial control osteopontin oligonucleotide”
cggatgtgatcgatagt

SEQ ID NO: 172 - DNA, 18 bp, Artificial Sequence
  source: /note=“Description of Artificial Sequence Artificial primer osteopontin”
acaagacatcaactgtgc

SEQ ID NO: 173 - DNA, 21 bp, Artificial Sequence
  source: /note=“Description of Artificial Sequence Artificial primer osteopontin”
tgacctcagaagatgaactct

SEQ ID NO: 174 - DNA, 20 bp, Artificial Sequence
  source: /note=“Description of Artificial Sequence Artificial primer GAPDH”
actttgtcaagctcatttcc

SEQ ID NO: 175 - DNA, 20 bp, Artificial Sequence
  source: /note=“Description of Artificial Sequence Artificial primer GAPDH”
tgcagcgaactttattgatg

SEQ ID NO: 176 - DNA, 30 bp, Artificial Sequence
  /note=“Description of Artificial Sequence Artificial Sense Control oligonucleotide”
gacagaaacaattcctcctgccgcatttac